SEQUENCE LISTING (80 sequences)

SEQ ID NO: 1 - protein, 6 aa, synthetic construct
SSYYSR

SEQ ID NO: 2 - protein, 6 aa, synthetic construct
SSYYSL

SEQ ID NO: 3 - protein, 7 aa, synthetic construct
RSSYYSL

SEQ ID NO: 4 - protein, 4 aa, synthetic construct
PLGL

SEQ ID NO: 5 - protein, 4 aa, synthetic construct
PIGI

SEQ ID NO: 6 - protein, 4 aa, synthetic construct
PYSI

SEQ ID NO: 7 - protein, 4 aa, synthetic construct
PYGI

SEQ ID NO: 8 - protein, 4 aa, synthetic construct
PYGL

SEQ ID NO: 9 - protein, 4 aa, synthetic construct
VLSL

SEQ ID NO: 10 - protein, 4 aa, synthetic construct
VYGL

SEQ ID NO: 11 - protein, 4 aa, synthetic construct
VLGL

SEQ ID NO: 12 - protein, 4 aa, synthetic construct
VYSL

SEQ ID NO: 13 - protein, 5 aa, synthetic construct
PISIY

SEQ ID NO: 14 - protein, 4 aa, synthetic construct
PSGL

SEQ ID NO: 15 - protein, 4 aa, synthetic construct
PLGI

SEQ ID NO: 16 - protein, 4 aa, synthetic construct
PMAL

SEQ ID NO: 17 - protein, 7 aa, synthetic construct
FPKFFSQ

SEQ ID NO: 18 - protein, 4 aa, synthetic construct
GFLG

SEQ ID NO: 19 - protein, 5 aa, synthetic construct
KPIEF

SEQ ID NO: 20 - protein, 8 aa, synthetic construct
GPLGIAGQ

SEQ ID NO: 21 - protein, 7 aa, synthetic construct
GPEGIWQ

SEQ ID NO: 22 - protein, 5 aa, synthetic construct
GGGRR

SEQ ID NO: 23 - protein, 7 aa, synthetic construct
APIGIAL

SEQ ID NO: 24 - protein, 8 aa, synthetic construct
APLGLYAL

SEQ ID NO: 25 - protein, 8 aa, synthetic construct
CKPISIAL

SEQ ID NO: 26 - protein, 8 aa, synthetic construct
CVLSLYAL

SEQ ID NO: 27 - protein, 7 aa, synthetic construct
VYGLIAL

SEQ ID NO: 28 - protein, 8 aa, synthetic construct
GPLSLSAL

SEQ ID NO: 29 - protein, 8 aa, synthetic construct
CPLGLYAL

SEQ ID NO: 30 - protein, 8 aa, synthetic construct
CPISIUVI

SEQ ID NO: 31 - protein, 8 aa, synthetic construct
CQPSGLAL

SEQ ID NO: 32 - protein, 8 aa, synthetic construct
GPYSIYAL

SEQ ID NO: 33 - protein, 9 aa, synthetic construct
CKPLGLWAL

SEQ ID NO: 34 - protein, 10 aa, synthetic construct
CRPMALILAL

SEQ ID NO: 35 - protein, 10 aa, synthetic construct
CQPLGALYAL

SEQ ID NO: 36 - protein, 11 aa, synthetic construct
CKPSGLSLAGI

SEQ ID NO: 37 - protein, 11 aa, synthetic construct
APSGLYALISL

SEQ ID NO: 38 - protein, 9 aa, synthetic construct
CQPLGLYAG

SEQ ID NO: 39 - protein, 9 aa, synthetic construct
CRPLGLWAL

SEQ ID NO: 40 - protein, 10 aa, synthetic construct
CPISIALGAL

SEQ ID NO: 41 - protein, 10 aa, synthetic construct
CPYSIYALGL

SEQ ID NO: 42 - protein, 9 aa, synthetic construct
CKVLGLIAL

SEQ ID NO: 43 - protein, 10 aa, synthetic construct
CGQVYGLAII

SEQ ID NO: 44 - protein, 9 aa, synthetic construct
CRVLGLIAL

SEQ ID NO: 45 - protein, 4 aa, synthetic construct
CFRL

SEQ ID NO: 46 - protein, 4 aa, synthetic construct
ALRG

SEQ ID NO: 47 - protein, 5 aa, synthetic construct
CVRGG

SEQ ID NO: 48 - protein, 7 aa, synthetic construct
CALRGAI

SEQ ID NO: 49 - protein, 8 aa, synthetic construct
IGFLGIGL

SEQ ID NO: 50 - protein, 8 aa, synthetic construct
AIGFLGAV

SEQ ID NO: 51 - protein, 8 aa, synthetic construct
CPGFLGIA

SEQ ID NO: 52 - protein, 10 aa, synthetic construct
CPFGFLGIAL

SEQ ID NO: 53 - protein, 9 aa, synthetic construct
CRPGFLGAL

SEQ ID NO: 54 - protein, 9 aa, synthetic construct
IAGFRGIAK

SEQ ID NO: 55 - protein, 8 aa, synthetic construct
PGFRKIAG

SEQ ID NO: 56 - protein, 8 aa, synthetic construct
CPVRIAKG

SEQ ID NO: 57 - protein, 8 aa, synthetic construct
CQPVRIAL

SEQ ID NO: 58 - protein, 8 aa, synthetic construct
CQFRVIAL

SEQ ID NO: 59 - protein, 9 aa, synthetic construct
CKPFRVGAL

SEQ ID NO: 60 - protein, 9 aa, synthetic construct
CRPAFLGVL

SEQ ID NO: 61 - protein, 9 aa, synthetic construct
CQPAFLGVL

SEQ ID NO: 62 - protein, 10 aa, synthetic construct
CIGVRGIAQL

SEQ ID NO: 63 - protein, 9 aa, synthetic construct
CPIKGAIWL

SEQ ID NO: 64 - protein, 10 aa, synthetic construct
CGQVYGLAIL

SEQ ID NO: 65 - protein, 8 aa, synthetic construct
CPLSLYAL

SEQ ID NO: 66 - protein, 8 aa, synthetic construct
CPLSLYSL

SEQ ID NO: 67 - protein, 7 aa, synthetic construct
CPLGLAL

SEQ ID NO: 68 - protein, 8 aa, synthetic construct
CAPIGIAL

SEQ ID NO: 69 - protein, 9 aa, synthetic construct
CQPLGLWAL

SEQ ID NO: 70 - protein, 7 aa, synthetic construct
CPISIAL

SEQ ID NO: 71 - protein, 9 aa, synthetic construct
  NON_STD <1: note = N-terminal acylation
CPIKGAIWL

SEQ ID NO: 72 - protein, 10 aa, synthetic construct
  NON_STD <1: note = N-terminal acylation
CGQVYGLAIL

SEQ ID NO: 73 - protein, 4 aa, synthetic construct
  NON_STD <1: note = N-terminal acylation
CFRL

SEQ ID NO: 74 - protein, 9 aa, synthetic construct
  NON_STD <1: note = N-terminal acylation
CKPLGLWAL

SEQ ID NO: 75 - protein, 9 aa, synthetic construct
  NON_STD <1: note = N-terminal acylation
CQPLGLWAL

SEQ ID NO: 76 - protein, 8 aa, synthetic construct
  NON_STD <1: note = N-terminal acylation
CPLGLYAL

SEQ ID NO: 77 - protein, 10 aa, synthetic construct
  NON_STD <1: note = N-terminal acylation
CPFGFLGIAL

SEQ ID NO: 78 - protein, 8 aa, synthetic construct
  NON_STD <1: note = N-terminal acylation
CKPLGLWA

SEQ ID NO: 79 - protein, 7 aa, synthetic construct
  NON_STD <1: note = N-terminal acylation
CPISIAL

SEQ ID NO: 80 - protein, 8 aa, synthetic construct
  NON_STD <1: note = N-terminal acylation
CAPIGIAL

(19) United States
(12) Patent Application Publication
(10) Pub. No.: US 2025/0127799 A1
(43) Pub. Date: Apr. 24, 2025

(54) DRUG CONJUGATES FOR BONE MARROW PROTECTION

(71) Applicant: ZYMERON CORPORATION, Research Triangle Park, NC (US)

(72) Inventors: Xu Li, Durham, NC (US); Zhiguo Zhou, Durham, NC (US); Jie Rong, Durham, NC (US); Dan Su, Durham, NC (US)

(21) Appl. No.: 18/777,277

(22) Filed: Jul. 18, 2024

Related U.S. Application Data
(60) Provisional application No. 63/514,530, filed on Jul. 19, 2023.

(51) Int. Cl. A61K 31/661; A61K 31/095; A61K 47/54; A61K 47/64; A61P 39/00; C07K 5/103; C07K 5/107

(52) U.S. Cl. CPC A61K 31/661; A61K 31/095; A61K 47/555; A61K 47/64; A61P 39/00; C07K 5/101; C07K 5/1016

(57) ABSTRACT

Bone marrow, the primary hematopoietic organ, is more sensitive than other organs when exposed to ionizing radiation or chemotherapeutic drugs due to the fragile hematopoietic stem and progenitor cells (HSPCs). Described herein are novel compounds, compositions, and methods comprising protective drug-based conjugates to achieve the specific protection of HSPCs in the bone marrow.

### CROSS-REFERENCE TO RELATED APPLICATIONS

This application claims priority to U.S. Provisional Patent Application No. 63/514,530, filed Jul. 19, 2023, which is incorporated by reference herein in its entirety.

### FEDERALLY SPONSORED RESEARCH

This invention was made with government support under contract number W81XWH22C0012 awarded by the Department of Defense. The government has certain rights in the invention.

### REFERENCE TO SEQUENCE LISTING

This application was filed with a Sequence Listing XML in ST.26 XML format accordance with 37 C.F.R. § 1.831 and PCT Rule 13ter. The Sequence Listing XML file submitted in the USPTO Patent Center, “221987-0005-US02_sequence_listing_xml_11-JUL-2024.xml,” was created on Jul. 11, 2024, contains 80 sequences, has a file size of 76.0 kilobytes (77,824 bytes), and is incorporated by reference in its entirety into the specification.

### TECHNICAL FIELD

Described herein are compounds, compositions, and methods relating to the fields of pharmaceutical sciences and clinical medicine. More particularly, the present invention relates to drug delivery platforms used in the prevention of bone marrow-related diseases. In some embodiments, the compounds, compositions, and methods may include bone-targeting ligands and protective drugs conjugated through cleavable peptides.

### BACKGROUND

Bone marrow, the primary hematopoietic organ, is more sensitive than other organs when exposed to ionizing radiation or chemotherapeutic drugs due to the fragile hematopoietic stem and progenitor cells (HSPCs). Relevant severe diseases include acute radiation syndrome (hematopoietic subsyndrome), radiotherapy-mediated myelotoxicity, and chemotherapy-caused myelosuppression. Currently, the United States Food and Drug Administration has approved four post-exposure therapeutics to treat these diseases—all of them are recombinant human growth factors that enhance the hematopoiesis in the bone marrow and reduce the infection risks (neutropenia or thrombocytopenia). Comparatively, it remains an emergent, unmet clinical need for pre-exposure prophylactic medicine that can prevent HSPCs from harmful impacts and thus improve survival, accelerate recovery, and reduce care costs.

Bone marrow protective drugs are pharmaceutical drugs and drug candidates that can provide protection to the bone marrow HSPCs from radiation damage or chemical toxicity can be categorized into three groups by their mechanisms of action: (1) scavengers of reactive oxygen or nitrogen species (RONS) and radicals that are caused by ionizing radiation exposure (Weiss et al., Toxicology 189 (1-2): 1-20 (2003)); (2) neutralizers of alkylating agents and chelators to platinum drugs that are widely used in cancer chemotherapy (Huber et al., Curr. Opin. Pharmacol. 7 (4): 404-409 (2007)); and (3) modulators of cell or tissue activities by activating or inhibiting specific pathways to enable cell resistance to the harmful effects of radiation or chemical compounds, e.g., Weiss et al., Ann. Oncology, 30 (10): 1613-1621 (2019).

Amifostine, also known as WR2721, is an extensively studied protective drug as its active thiol metabolite, amifostine thiol (WR1065), is able to scavenge radicals, neutralize alkylating agents and platinum drugs, and stabilize the DNA double strands. The initial goal for the development of this protective aminothiol compound was to protect against acute radiation syndrome, and amifostine was selected among numerous cysteamine (NH₂CH₂CH₂SH) derivatives due to its demonstrated radioprotective efficacy. Singh et al., Expert Opin. Drug Saf. 18 (11): 1077-1090 (2019). Subsequently, this drug was investigated for protecting multiple organs (such as bone marrow, kidneys, etc.) in patients undergoing chemotherapy.

Currently, amifostine is approved for limited use (e.g., salivary glands and kidneys) in patients with specific cancer types undergoing chemotherapy or radiotherapy. The protective effects of amifostine for HSPCs in the bone marrow are known. However, amifostine has been considered unsuitable for clinical use because of its unfavorable pharmacokinetic profile—a narrow time window of administration for optimal efficacy (e.g., must be administrated 15-30 min before irradiation or chemotherapeutic administration) and very short duration of effect due to its short pharmacokinetic circulating time (<8 min) and short bone marrow residence time (<30 min). Also, amifostine frequently leads to side effects such as hypotension when administered at the doses required to achieve bone marrow protection. Therefore, novel pharmaceutical formulation strategies that overcome these constraints could make amifostine fully effective and gain its clinical advantages and benefits as a pre-exposure prophylactic medicine to protect HSPCs in bone marrow from damage.

Several attempted formulations of amifostine have previously been reported to improve its performance as a systemic protective drug. However, these formulations are not suitable or effective for clinical translation, especially for bone marrow protection, because of their poor tissue distribution and accumulation in the bone marrow.

What is needed are novel compounds, compositions, and methods for effectively targeting drugs to the bone marrow that are capable of protecting the HSPCs in the bone marrow from harmful radiation and chemotherapeutic toxicity.

### SUMMARY

One embodiment described herein is a drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.

In one aspect, at least one of the bone marrow protective drug moieties is a bone marrow protective drug moiety of formula (I):

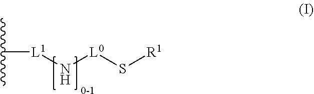

wherein:

R¹ is hydrogen or —PO₃(R^{1a})₀₋₂, wherein —PO₃(R^{1a})₀₋₂ has a net charge of 0, —1, or —2;

R^{1a}, at each occurrence, is independently hydrogen, Na⁺, K⁺, Ca⁺², Mg⁺², or NH₄⁺;

L⁰ and L¹ are each independently a C₁₋₂₄alkylene wherein optionally 1-8 methylene groups in the C₁₋₂₄alkylene are each independently replaced with —N(RX)—, —O—, —S-,

—SO—, —SO₂-, or —C(O)-, wherein 2 methylene groups replaced with —N(R^{X})-,

—O—, —S—, —SO—, or —SO₂- are separated by two or more carbon atoms in the alkylene and/or optionally 1-4 methylene groups in the C₁₋₂₄alkylene is replaced with —Cy-;

Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the bone marrow protective drug moiety of formula (I) is a moiety of formula (I-a):

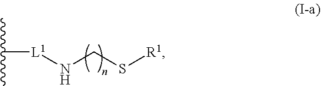

wherein n=1-24.

In another aspect, R¹ is hydrogen, —PO₃⁻², —PO₃H⁻, or —PO₃H².
In another aspect, L¹ comprises —N(H)-.
In another aspect, L¹ is —C₁₋₄alkylene -N(H)-.
In another aspect, n is 1-4.
In another aspect, the bone marrow protective drug moiety is

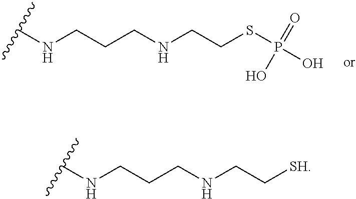 [SMILES: *NCCCNCCSP(=O)(O)O ; "or" ; *NCCCNCCS]

In another aspect, the first linker conjugates to the first terminus of the cleavable peptide moiety by a chemical bond selected from the group consisting of an amide bond, an ester bond, a sulfur-carbon single bond, a disulfide bond, a nitrogen-carbon single bond, a nitrogen-carbon double bond, and a nitrogen-nitrogen double bond.
In another aspect, in the first linker connects to at least one bone marrow protective drug moiety by a linkage selected from the group consisting of

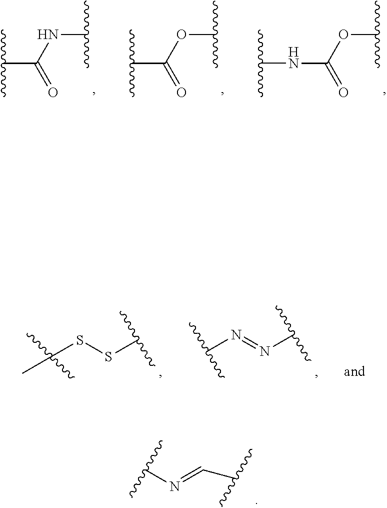

In another aspect, the first linker comprises one cleavable chemical bond between one bone marrow protective drug moiety and the first terminus of the cleavable peptide moiety.
In another aspect, the bond is an amide bond between L¹ of the bone marrow protective drug moiety and the first terminus of the cleavable peptide moiety.
In another aspect, the cleavable peptide moiety comprises less than 30 amino acids, and the amino acids are in a linear, cyclic, or branched arrangement.
In another aspect, the cleavable peptide moiety has a molecular weight of less than 3,500 Daltons.
In another aspect, the cleavable peptide moiety is a moiety of formula (II-A), (II-B), (II-C) or (II-D):

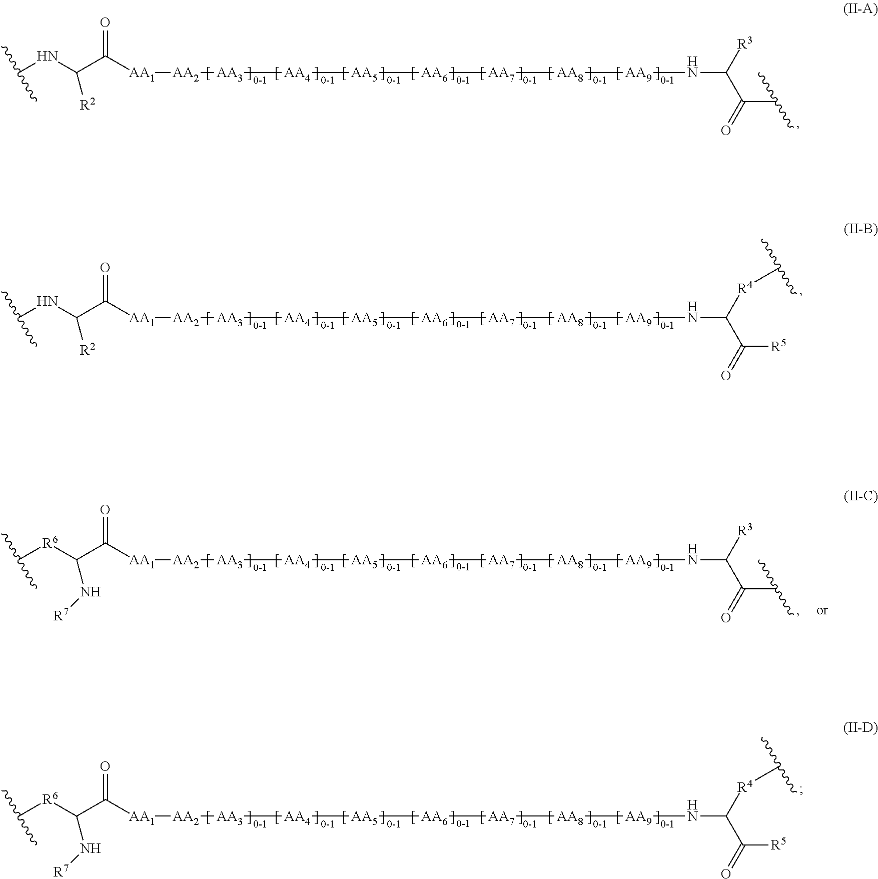

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, Y, P, Q, or V;
AA₈, at each occurrence is A, I, S, N, V, G, R, M, or Q;
AA₉, at each occurrence, is G, or S, A, L, V, W, Y, N;

R² and R³, at each occurrence, are each independently hydrogen, —CH₃,

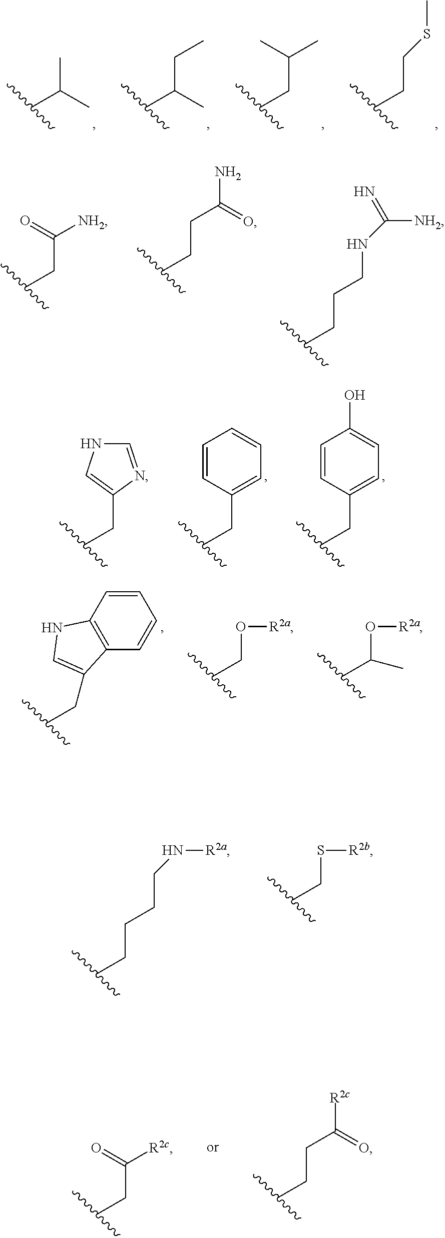

wherein:
R^{2a}, at each occurrence, is hydrogen or

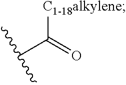

R^{2b}, at each occurrence, is hydrogen,

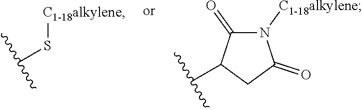

and
R^{2c}, at each occurrence, is hydrogen,

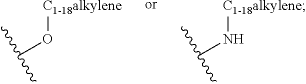

R⁴ and R⁶, at each occurrence, are each independently

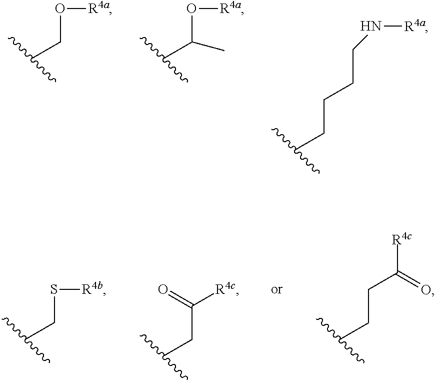

wherein:
R^{4a} is

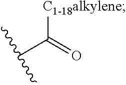

R^{4b} is

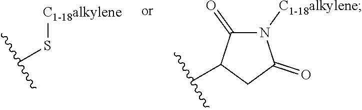

and
R^{4c} is

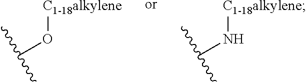

R⁵ is —OH, —O—C₁₋₁₈alkylene, or —NH—C₁₋₁₈alkylene, wherein optionally 1-6 methylene groups in the C₁₋₁₈alkylene are independently replaced with —N(R^{X})—, —O—, —S—, —SO—, —SO₂—, or —C(O)—, wherein 2 methylene groups replaced with —N(R^{X})—, —O—, —S—, —SO—, or —SO₂- are separated by two or more carbon atoms in the alkylene and/or optionally 1-4 methylene groups in the alkylene of the C₁₋₁₈alkylene is replaced with —Cy-;

R⁷ is hydrogen, or —C(O)—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of the C₁₋₁₈alkylene is replaced with —Cy-;

Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the cleavable peptide moiety is a moiety of formula (II-C-A):

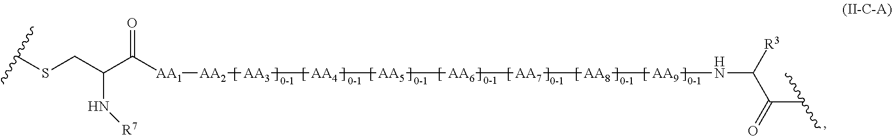

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;

R³ is hydrogen, —CH₃,

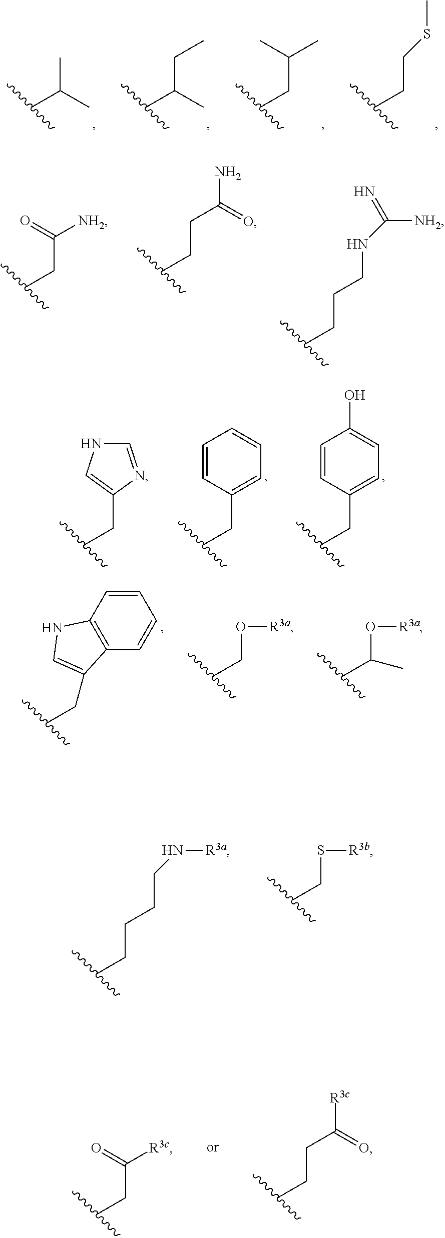

wherein:
R^{3a}, at each occurrence, is hydrogen or

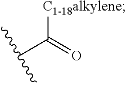

R^{3b}, at each occurrence, is hydrogen,

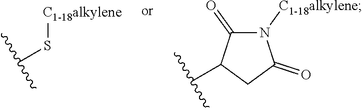

and
R^{3c}, at each occurrence, is hydrogen,

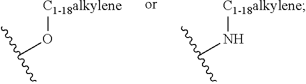

R⁷ is hydrogen, or —C(O)—C₁₋₁₈alkylene, wherein optionally 1-6 methylene groups in the C₁₋₁₈alkylene are independently replaced with —N(RX)—, —O—, —S—, —SO—, —SO₂-, or —C(O)-, wherein 2 methylene groups replaced with —N(RX)—, —O—, —S—, —SO-, or —SO₂- are separated by two or more carbon atoms in the alkylene and/or optionally 1-4 methylene groups in the C₁₋₁₈alkylene is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the cleavable peptide moiety is a moiety of formula (III):

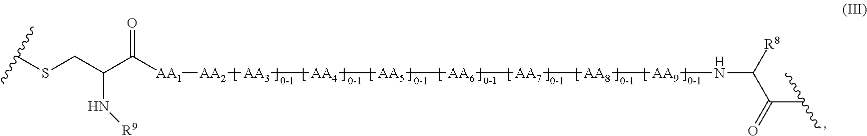

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or l;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;

R⁸ is hydrogen, —CH₃,

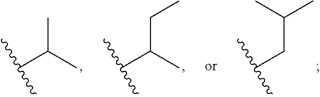

and

R⁹ is hydrogen or —C(O)CH₃.

In another aspect, the cleavable peptide moiety is a moiety of formula (III-A) or (III-B):

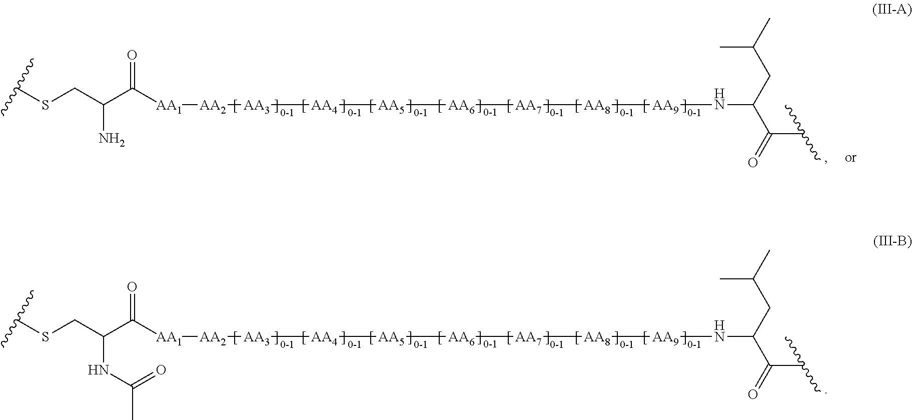

In another aspect, the cleavable peptide moiety comprises an amino acid sequence of any one of SEQ ID NO: 1-80.
In another aspect, the cleavable peptide moiety comprises at least one sequence of PLGL (SED ID NO: 4), PIGI (SED ID NO: 5), PYSI (SED ID NO: 6), PYGI (SED ID NO: 7), PYGL (SED ID NO: 8), VLSL (SED ID NO: 9), VYGL (SED ID NO: 10), VLGL (SEQ ID NO: 11); VYSL (SED ID NO: 12), PISIY (SED ID NO: 13), PSGL (SED ID NO: 14), PLGI (SED ID NO: 15), or PMAL (SED ID NO: 16).
In another aspect, the first terminus of the cleavable peptide moiety is the carboxy (C) terminus of the cleavable peptide moiety, and the second terminus of the cleavable peptide moiety is the amino (N) terminus of the cleavable peptide moiety.
In another aspect, the cleavable peptide moiety is cleavable by one or more bone marrow-enriched proteases.
In another aspect, at least one bone marrow-enriched protease is a matrix metalloprotease, a disintegrin and metalloproteinase, a cathepsin B protease, a cathepsin K protease, a urokinase plasminogen activator protease, or a tissue-plasminogen activator protease.
In another aspect, the bone-targeting ligand moiety is a bisphosphonate moiety of formula (IV):

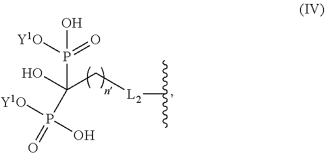

wherein:

Y¹, at each occurrence, is independently hydrogen, Na⁺, K⁺, Ca⁺², Mg⁺², or NH₄⁺;

n′is 1-16;

L² is a C₁₋₂₄alkylene wherein optionally 1-8 methylene groups in the alkylene of L² are independently replaced with —N(R^{X})—, —O—, —S—, —SO—, —SO2—, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of L² is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, Y¹, at each occurrence, is independently hydrogen, Na⁺, K⁺, Ca⁺², Mg⁺², or NH⁴⁺.
In another aspect, n′ is 0-4.
In another aspect, L² comprises —N(H)—, —N(H)—C₁₋₄alkylene-, —N(CH₃)-C₁₋₄alkylene-, phenylene, imidazolylene, or triazolylene.
In another aspect, the bone-targeting ligand moiety is

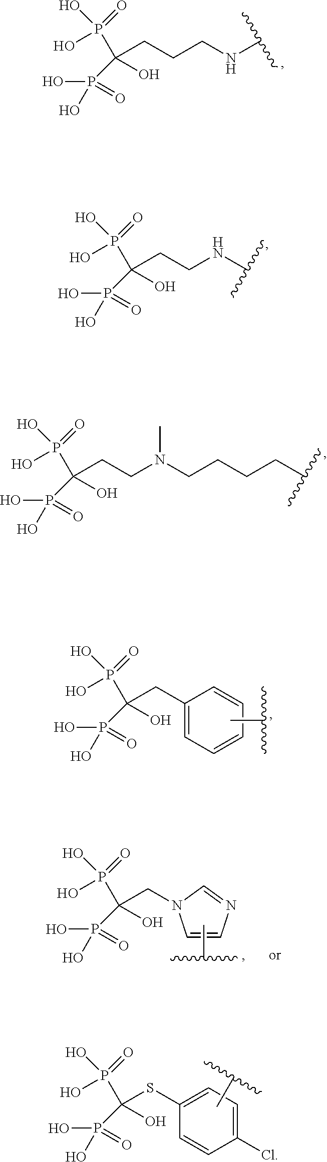

In another aspect, the second linker connects to bisphosphonate moiety by a linkage selected from the group consisting of

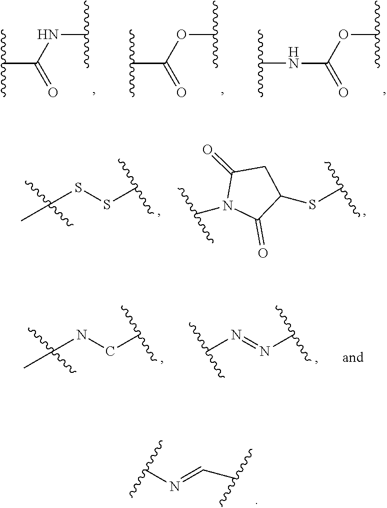

In another aspect, the second linker is a —C₁₋₂₄alkylene-wherein optionally 1-8 methylene groups in the second linker are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the second linker is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the second linker comprises a maleimide moiety, wherein the maleimide moiety is attached to a cysteine moiety that is at least two amino acids away from the first terminus of the cleavable peptide moiety.
In another aspect, the second linker is a linker having formula:

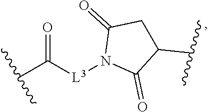

wherein:

L³ is a C₁₋₁₈alkylene wherein optionally 1-6 methylene groups in the alkylene of L³ are independently replaced with —N(R^{X})—, —O—, —S—, —SO—, —SO₂-, or-C (O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of L³ is replaced with —Cy-;

Cy, at each occurrence, is a C₃₋₆cycloalkylene, a phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

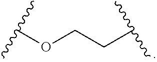 [SMILES: *CCO*]

In another aspect, L³ comprises repeating units of
In another aspect, the second linker is:

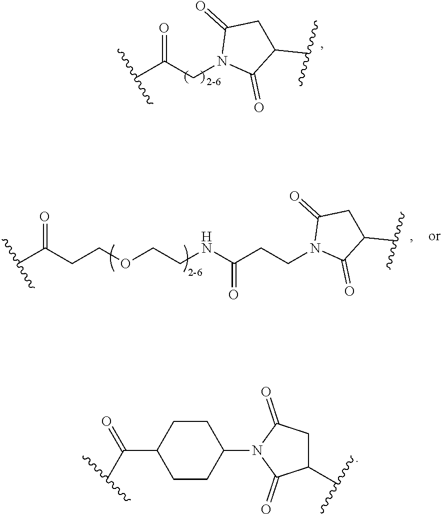

Another embodiment described herein is a pharmaceutical composition comprising the drug conjugate compound described herein and a pharmaceutically acceptable carrier.

Another embodiment described herein is a method of targeted delivery of a protective drug to bone marrow in a subject, the method comprising administering to the subject a therapeutically effective amount of the drug conjugate compound described herein or a pharmaceutical composition thereof.

Another embodiment described herein is a method of targeted delivery of a protective drug to bone marrow in a subject, the method comprising:

administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid,
where the amino acid conjugated to the bone-targeting ligand moiety is at least two amino acids away from the first terminus of the cleavable peptide moiety.

Another embodiment described herein is a method of protecting a subject from radiation or chemotherapeutic toxicity, the method comprising:

administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid,
where the amino acid conjugated to the bone-targeting ligand moiety is at least
two amino acids away from the first terminus of the cleavable peptide moiety.

Another embodiment described herein is a method of treating or preventing a bone marrow disease in a subject, the method comprising:

administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid,
where the amino acid conjugated to the bone-targeting ligand moiety is at least two amino acids away from the first terminus of the cleavable peptide moiety.
   In one aspect, the bone marrow disease comprises ionizing radiation-induced acute hematopoietic subsyndrome, chemotherapy-caused myelosuppression, radiotherapy-induced myelotoxicity, or combinations thereof.

Another embodiment described herein is a method of synthesizing a drug conjugate compound, the method comprising:

adding one or more protective moieties to a peptide using 2,2′-dipyridyldisulfide and N-(9-fluorenylmethoxycarbonyloxy) succinimide to generate a protected peptide;

activating the carboxy terminus of the protected peptide using N-ethyl-N-(3-(dimethylamino)propyl) carbodiimide and N-hydroxysuccinimide (NHS) to generate a peptide —NHS ester;

adding a bone marrow protective drug to the peptide-NHS ester using acetonitrile to generate a drug-conjugated peptide;

removing the one or more protective moieties from the drug-conjugated peptide using dithiothreitol and piperidine;

functionalizing a bone-targeting ligand using N-succinimidyl 6-maleimidohexanoate; and adding the functionalized bone-targeting ligand to the drug-conjugated peptide to generate the drug conjugate compound.

Another embodiment described herein is a kit for targeted delivery of a protective drug to bone marrow in a subject, the kit comprising:

a pharmaceutical composition comprising a drug conjugate compound comprising: one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety;

optionally, one or more pharmaceutically acceptable buffers, carriers, or excipients; and

optionally, one or more of packaging or instructions for use.

Another embodiment described herein is the use of a drug conjugate compound for targeted delivery of a protective drug to bone marrow in a subject, the drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.

Another embodiment described herein is the use of a drug conjugate compound for protecting a subject from radiation or chemotherapeutic toxicity, the drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.

Another embodiment described herein is the use of a drug conjugate compound for treating or preventing a bone marrow disease in a subject, the drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.

### DESCRIPTION OF THE DRAWINGS

FIG. 1 shows a general illustrative structure for a bone marrow-specific protective drug conjugate comprising a protective drug, a protease-cleavable peptide, and a bone-targeting ligand. The protective drug and the protease-cleavable peptide may be conjugated through a first linker (Linker 1), and the protease-cleavable peptide and the bone-targeting ligand may be conjugated through a second linker (Linker 2).

FIG. 2A-B show the HPLC (FIG. 2A) and the LC/MS (FIG. 2B) spectra of an Alendronate-AcCPIKGAIWL-Amifostine conjugate.

FIG. 3A-F show the in vivo radioprotective effects of two bone marrow-specific protective drug conjugates having different peptide sequences. CD2F1 male mice (n=16) were injected subcutaneously with the different doses of Conjugate 1 at 6 h prior to the exposure of 9.2 Gy y-irradiation (FIG. 3A), Conjugate 2 at 24 h prior to the exposure of 9.2 Gy y-irradiation (FIG. 3B), Conjugate 1 at 9 h prior to the exposure of 9.2 Gy y-irradiation (FIG. 3C), Conjugate 1 at 12 h prior to the exposure of 9.2 Gy y-irradiation (FIG. 3D), Conjugate 1 at 12 h prior to the exposure of 9.1 Gy y-irradiation (FIG. 3E), Conjugate 1 with hydrolyzed thiol-maleimide moiety at 6 h prior to the exposure of 9.2 Gy y-irradiation (FIG. 3F). Survival was monitored for 30 days post-irradiation. *** Indicates a significant difference compared to the vehicle control (p<0.001).

FIG. 4A shows the proteolytic cleavage of three conjugates having different peptide sequences in human bone marrow aspirate from the same donor over a 6 h time period. FIG. 4B shows the proteolytic cleavage of Conjugate 3 in human bone marrow aspirates from two different donors over a 6 h time period.

FIG. 5 shows the binding kinetics of a drug conjugate having the peptide sequence AcCPISIAL to hydroxyapatite powder.

FIG. 6A-B show the comparable radioprotective efficacy of a protective conjugate and amifostine alone to human HSPCs. FIG. 6A shows the viability of human HSPCs exposed to ionizing radiation of 3 Gy or 8 Gy, with or without pre-treatment of the conjugate or amifostine at 0, 10, 20, 50, or 100 μM. FIG. 6B shows the viability of human HSPCs pre-treated with the conjugate or amifostine alone (at a fixed concentration of 50 M), followed by irradiation with different doses of 2, 4, 6, or 8 Gy. In both experiments, N=6; * indicates a P value <0.05 as compared to the Control group without using radioprotective agents.

FIG. 7 shows the matrix metalloproteinase-based proteolytic cleavage of three conjugates having different peptide sequences. The control group was Conjugate 1 incubated at the same conditions without adding matrix metalloproteinase.

FIG. 8A-B show the protective effects of the conjugate on the human bone marrow mononuclear cells exposed to the chemodrug carboplatin. In FIG. 8A, Amifostine and WR1065 were included as controls. The y-axis represents the percentage of CFU growth in the treatment groups normalized to the untreated control (considered as 100% of growth). For each group, N=2. In FIG. 8B, Amifostine was included as a control. For each group, N=4. *, **, and *** indicate a significant difference compared to the vehicle control (p<0.05, 0.01, and 0.001, respectively).

FIG. 9 shows the enhancement of the proliferation and differentiation of human bone marrow mononuclear cells exposed to the conjugate or other protective drugs, i.e., Amifostine or WR1065. The y-axis represents the percentage of CFU growth in the treatment groups normalized to the untreated control (considered as 100% growth). For each group, N=2.

FIG. 10A-D show the 72-hour proliferation of cancer cells, A549 lung cancer cells (FIG. 10A-B) or Hs578T breast cancer cells (FIGS. 10C-D), exposed to chemodrugs, Carboplatin (FIG. 10A and FIG. 10C) or Paclitaxel (FIG. 10B and FIG. 10D) with or without the presence of the Conjugate. Amifostine was included as a control. For each group, N=4-5. * indicates P<0.05.

FIG. 11A-D show the representative histological images for bone marrow (FIG. 11A-B) and spleen (FIG. 11C-D) in survived mice after 30 days undergoing the 9.2 Gy y-irradiation. In FIG. 11A, black arrows indicate the pools of hemorrhage. In FIG. 11C-D, white arrows indicate the white pulp areas.

### DETAILED DESCRIPTION

Unless otherwise defined, all technical and scientific terms used herein have the same meaning as commonly understood by one of ordinary skill in the art. For example, any nomenclatures used in connection with, and techniques of chemistry, biochemistry, molecular biology, immunology, microbiology, genetics, cell and tissue culture, and protein and nucleic acid chemistry described herein are well-known and commonly used in the art. In case of conflict, the present disclosure, including definitions, will control. Exemplary methods and materials are described below, although methods and materials similar or equivalent to those described herein can be used in practice or testing of the embodiments and aspects described herein.

As used herein, the terms “amino acid,” “nucleotide,” “polynucleotide,” “vector,” “polypeptide,” and “protein” have their common meanings as would be understood by a biochemist of ordinary skill in the art. Standard single-letter nucleotides (A, C, G, T, U) and standard single-letter amino acids (A, C, D, E, F, G, H, I, K, L, M, N, P, Q, R, S, T, V, W, or Y) are used herein.

As used herein, the terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not.

As used herein, the term “a,” “an,” “the,” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “˜” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the terms “control,” or “reference” are used herein interchangeably. A “reference” or “control” level may be a predetermined value or range, which is employed as a baseline or benchmark against which to assess a measured result. “Control” also refers to control experiments or control cells.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount” refer to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human. As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of a biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after the clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifested.

As used herein, the terms “radiation” or “radiation therapy” refer to ionizing radiation rays, nuclear rays, or radionucleotide particles used to treat a subject, e.g., a subject having cancer. X-rays, gamma rays, or charged particles (e.g., protons or electrons) may be used to generate ionizing radiation. Radiation therapy may be delivered to a subject by a machine placed outside the subject's body (external-beam radiation therapy), by a source placed inside the subject's body (internal radiation therapy or brachytherapy), or through systemic radioisotopes delivered intravenously or orally to the subject (systemic radioisotope therapy).

Definitions of specific functional groups and chemical terms are described in more detail herein. The chemical elements are identified in accordance with the Periodic Table of the Elements, CAS version, Handbook of Chemistry and Physics, 75^{th} ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} ed, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} ed, Cambridge University Press, Cambridge, 1987.

As used herein, the term “alkyl” refers to a straight or branched hydrocarbon radical having from 1 to 12 (e.g., C₁-C₁₂) carbon atoms and includes, for example, methyl, ethyl, n-propyl, isopropyl, n-butyl, sec-butyl, isobutyl, tert-butyl, n-pentyl, iso-pentyl, n-hexyl, and the like.

As used herein, the term “alkenyl” refers to straight and branched hydrocarbon radicals having from 2 to 12 carbon atoms (e.g., C₂-C₁₂) and at least one double bond and includes, but is not limited to, ethenyl, 3-buten-1-yl, 2-ethenylbutyl, 3-hexen-1-yl, and the like. The term “alkenyl” includes cycloalkenyl, and heteroalkenyl in which 1 to 3 heteroatoms selected from O, S, N, or substituted nitrogen may replace carbon atoms.

As used herein, the term “alkynyl” refers to straight and branched hydrocarbon radicals having from 2 to 12 carbon atoms (e.g., C₂-C₁₂) and at least one triple bond and includes, but is not limited to, ethynyl, 3-butyn-1-yl, propynyl, 2-butyn-1-yl, 3-pentyn-1-yl, and the like.

As used herein, the term “cycloalkyl” refers to a monocyclic or polycyclic hydrocarbyl group having from 3 to 8 carbon atoms (e.g., C₃-C₈), for instance, cyclopropyl, cycloheptyl, cyclooctyl, cyclodecyl, cyclobutyl, adamantyl, norpinanyl, decalinyl, norbornyl, cyclohexyl, and cyclopentyl. Such groups can be substituted with groups such as hydroxy, keto, amino, alkyl, and dialkylamino, and the like. Also included are rings in which 1 to 3 heteroatoms replace carbons. Such groups are termed “heterocyclyl,” which means a cycloalkyl group also bearing at least one heteroatom selected from O, S, N, or substituted nitrogen. Examples of such groups include, but are not limited to, oxiranyl, pyrrolidinyl, piperidyl, tetrahydropyran, and morpholine.

As used herein, the term “alkoxy” refers to a straight or branched chain alkyl groups having 1-10 carbon atoms (e.g., C₂-C₁₀) and linked through oxygen. Examples of such groups include, but are not limited to, methoxy, ethoxy, propoxy, isopropoxy, n-butoxy, sec-butoxy, tert-butoxy, pentoxy, 2-pentyloxy, isopentoxy, neopentoxy, hexoxy, 2-hexoxy, 3-hexoxy, and 3-methylpentoxy. In addition, alkoxy refers to polyethers such as —O—(CH₂)₂—O—CH₃, and the like.

The alkyl, alkenyl, alkoxy, and alkynyl groups described herein are optionally substituted (i.e., may be substituted, but are not necessarily substituted), preferably by 1 to 3 groups selected from NR₄R_{5,} phenyl, substituted phenyl, thio C₁-C₆ alkyl, C₁-C₆ alkoxy, hydroxy, carboxy, C₁-C₆alkoxycarbonyl, halo, nitrile, cycloalkyl, and a 5-or 6-membered carbocyclic ring or heterocyclic ring having 1 or 2 heteroatoms selected from nitrogen, substituted nitrogen, oxygen, and sulfur. “Substituted nitrogen” means nitrogen bearing C1-C6 alkyl or (CH₂) pPh where p is 1, 2, or 3. Perhalo and polyhalo substitution is also included.

Examples of substituted alkyl groups include, but are not limited to, 2-aminoethyl, 2-hydroxyethyl, pentachloroethyl, trifluoromethyl, 2-diethylaminoethyl, 2-dimethylaminopropyl, ethoxycarbonylmethyl, 3-phenylbutyl, methanylsulfanylmethyl, methoxymethyl, 3-hydroxypentyl, 2-carboxybutyl, 4-chlorobutyl, 3-cyclopropylpropyl, pentafluoroethyl, 3-morpholinopropyl, piperazinylmethyl, and 2-(4-methylpiperazinyl) ethyl.

Examples of substituted alkynyl groups include, but are not limited to, 2-methoxyethynyl, 2-ethylsulfanylethynyl, 4-(1-piperazinyl)-3-(butynyl), 3-phenyl-5-hexynyl, 3-diethylamino-3-butynyl, 4-chloro-3-butynyl, 4-cyclobutyl-4-hexenyl, and the like.

Typical substituted alkoxy groups include aminomethoxy, trifluoromethoxy, 2-diethylaminoethoxy, 2-ethoxycarbonylethoxy, 3-hydroxypropoxy, 6-carboxhexyloxy, and the like.

Further, examples of substituted alkyl, alkenyl, and alkynyl groups include, but are not limited to, dimethylaminomethyl, carboxymethyl, 4-dimethylamino-3-buten-1-yl, 5-ethylmethylamino-3-pentyn-1-yl, 4-morpholinobutyl, 4-tetrahydropyrinidylbutyl, 3-imidazolidin-1-ylpropyl, 4-tetrahydrothiazol-3-yl-butyl, phenylmethyl, 3-chlorophenylmethyl, and the like.

As used herein, the term “anion” means a negatively charged species such as chloride, bromide, trifluoroacetate, or triethylammonium. The term “cation” refers to a positively charged species, such as sodium, potassium, or ammonium.

As used herein, the term “acyl” refers to alkyl or aryl (Ar) group having from 1-10 carbon atoms bonded through a carbonyl group, i.e., R—C(O)-. For example, acyl includes, but is not limited to, a C₁-C₆ alkanoyl, including substituted alkanoyl, wherein the alkyl portion can be substituted by an amine, amide, carboxylic, or heterocyclic group. Typical acyl groups include acetyl, benzoyl, and the like.

As used herein, the term “aryl” refers to an aromatic monocyclic hydrocarbon ring system or a polycyclic ring system where at least one of the rings in the ring system is an aromatic hydrocarbon ring and any other aromatic rings in the ring system include only hydrocarbons. In some embodiments, a monocyclic aryl group can have from 6 to 14 carbon atoms and a polycyclic aryl group can have from 8 to 14 carbon atoms (e.g., C₈-C₁₄). The aryl group can be covalently attached to the defined chemical structure at any carbon atom(s) that result in a stable structure. In some embodiments, an aryl group can have only aromatic carbocyclic rings, e.g., phenyl, 1-naphthyl, 2-naphthyl, anthracenyl, phenanthrenyl groups, and the like. In other embodiments, an aryl group can be a polycyclic ring system in which at least one aromatic carbocyclic ring is fused (i.e., having a bond in common with) to one or more cycloalkyl or cycloheteroalkyl rings. Examples of such aryl groups include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like.

As used herein, the terms “halogen” or “halo” refer to fluorine, bromine, chlorine, or iodine. As used herein, the term “haloalkyl” refers to an alkyl group having one or more halogen substituents. In some embodiments, a haloalkyl group can have 1 to 10 carbon atoms (e.g., C₁-C₈). Examples of haloalkyl groups include CF₃, C₂F₅, CHF₂, CH₂F, CCl₃, CHCl₂, CH₂Cl, C₂Cl₅, and the like. Perhaloalkyl groups, i.e., alkyl groups wherein all the hydrogen atoms are replaced with halogen atoms (e.g., CF₃ and C₂F₅), are included within the definition of “haloalkyl.” For example, a C₁₋₁₀ haloalkyl group can have the formula —CᵢH₂ᵢ+1−jXⱼ, wherein X is F, CI, Br, or I, i is an integer in the range of 1 to 10, and jis an integer in the range of 0 to 21, provided that j is less than or equal to 2i+1.

As used herein, the term “heteroaryl” refers to an aromatic monocyclic ring system containing at least one ring heteroatom selected from O, N, and S or a polycyclic ring system where at least one of the rings in the ring system is aromatic and contains at least one ring heteroatom. A heteroaryl group can have from 5 to 14 ring atoms (e.g., C₅-C₁₄), and contains 1-6 ring heteroatoms (e.g., N, O, S, P, or the like). In some embodiments, heteroaryl groups can include monocyclic heteroaryl rings fused to one or more aromatic carbocyclic rings, non-aromatic carbocyclic rings, or non-aromatic cycloheteroalkyl rings. The heteroaryl group can be covalently attached to the defined chemical structure at any heteroatom or carbon atom that results in a stable structure. Generally, heteroaryl rings do not contain O—O, S—S, or S—O bonds. However, one or more N or S atoms in a heteroaryl group can be oxidized (e.g., pyridine N-oxide, thiophene S-oxide, thiophene S,S-dioxide). Examples of such heteroaryl rings include pyrrolyl, furyl, thienyl, pyridyl, pyrimidyl, pyridazinyl, pyrazinyl, triazolyl, tetrazolyl, pyrazolyl, imidazolyl, isothiazolyl, thiazolyl, thiadiazolyl, isoxazolyl, oxazolyl, oxadiazolyl, indolyl, isoindolyl, benzofuryl, benzothienyl, quinolyl, 2-methylquinolyl, isoquinolyl, quinoxalyl, quinazolyl, benzotriazolyl, benzimidazolyl, benzothiazolyl, benzisothiazolyl, benzisoxazolyl, benzoxadiazolyl, benzoxazolyl, cinnolinyl, 1H-indazolyl, 2H-indazolyl, indolizinyl, isobenzofuyl, naphthyridinyl, phthalazinyl, pteridinyl, purinyl, oxazolopyridinyl, thiazolopyridinyl, imidazopyridinyl, furopyridinyl, thienopyridinyl, pyridopyrimidinyl, pyridopyrazinyl, pyridopyrdazinyl, thienothiazolyl, thienooxazolyl, thienoimidazolyl groups, and the like. Further examples of heteroaryl groups include 4,5,6,7-tetrahydroindolyl, tetrahydroquinolinyl, benzothienopyridinyl, benzofuropyridinyl groups, and the like.

As used herein, the term “lower alkenyl” refers to alkenyl groups which contains 2 to 6 carbon atoms (e.g., C₂-C₆). An alkenyl group is a hydrocarbyl group containing at least one carbon-carbon double bond. As defined herein, it may be unsubstituted or substituted with the substituents described herein. The carbon-carbon double bonds may be between any two carbon atoms of the alkenyl group. It is preferred that it contains 1 or 2 carbon-carbon double bonds and more preferably one carbon-carbon double bond. The alkenyl group may be straight chained or branched. Examples include but are not limited to ethenyl, 1-propenyl, 2-propenyl, 1-butenyl, 2-butenyl, 2-methyl-1-propenyl, 1,3-butadienyl, and the like.

As used herein, the term “lower alkynyl” refers to an alkynyl group containing 2-6 carbon atoms (e.g., C₂-C₆). An alkynyl group is a hydrocarbyl group containing at least one carbon-carbon triple bond. The carbon-carbon triple bond may be between any two-carbon atom of the alkynyl group. In an embodiment, the alkynyl group contains 1 or 2 carbon-carbon triple bonds and more preferably one carbon-carbon triple bond. The alkynyl group may be straight chained or branched. Examples include but are not limited to ethynyl, 1-propynyl, 2-propynyl, 1-butynyl, 2-butynyl and the like.

As used herein, the term “carbalkoxy” refers to an alkoxycarbonyl group, where the attachment to the main chain is through the carbonyl group, e.g., —C(O)-. Examples include but are not limited to methoxy carbonyl, ethoxy carbonyl, and the like.

As used herein, the term “oxo” refers to a double-bonded oxygen (i.e., ═O). It is also to be understood that the terminology C(O) refers to a —C═O group, whether it be ketone, aldehyde or acid or acid derivative. Similarly, S(O) refers to a —S═O group.

As used herein, the term “cycloalkyl” refers to a non-aromatic carbocyclic group including cyclized alkyl, alkenyl, and alkynyl groups. A cycloalkyl group can be monocyclic (e.g., cyclohexyl) or polycyclic (e.g., containing fused, bridged, and/or spiro ring systems), wherein the carbon atoms are located inside or outside of the ring system. A cycloalkyl group can have from 3 to 14 ring atoms (e.g., from 3 to 8 carbon atoms for a monocyclic cycloalkyl group and from 7 to 14 carbon atoms for a polycyclic cycloalkyl group). Any suitable ring position of the cycloalkyl group can be covalently linked to the defined chemical structure. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcaryl, adamantyl, and spiro [4.5] decanyl groups, as well as their homologs, isomers, and the like.

As used herein, the term “heteroatom” refers to an atom of any element other than carbon or hydrogen and includes, for example, nitrogen, oxygen, sulfur, phosphorus, and selenium.

As used herein, the term “cycloheteroalkyl” refers to a non-aromatic cycloalkyl group that contains at least one (e.g., one, two, three, four, or five) ring heteroatom selected from O, N, and S, and optionally contains one or more (e.g., one, two, or three) double or triple bonds. A cycloheteroalkyl group can have from 3 to 14 ring atoms and contains from 1 to 5 ring heteroatoms (e.g., from 3-6 ring atoms for a monocyclic cycloheteroalkyl group and from 7 to 14 ring atoms for a polycyclic cycloheteroalkyl group). The cycloheteroalkyl group can be covalently attached to the defined chemical structure at any heteroatom(s) or carbon atom(s) that results in a stable structure. One or more N or S atoms in a cycloheteroalkyl ring may be oxidized (e.g., morpholine N-oxide, thiomorpholine S-oxide, thiomorpholine S,S-dioxide). Cycloheteroalkyl groups can also contain one or more oxo groups, such as phthalimidyl, piperidinyl, oxazolidinoxyl, 2,4(1H,3H)-dioxo-pyrimidinyl, pyridin-2(1H)-onyl, and the like. Examples of cycloheteroalkyl groups include, among others, morpholinyl, thiomorpholinyl, pyranyl, imidazolidinyl, imidazolinyl, oxazolidinyl, pyrazolidinyl, pyrazolinyl, pyrrolidinyl, pyrrolinyl, tetrahydrofuranyl, tetrahydrothienyl, piperidinyl, piperazinyl, azetidine, and the like.

### Compounds

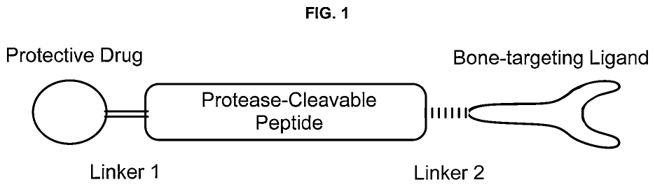

Described herein are a class of drug conjugate compounds that can induce the accumulation of cytoprotective drugs to the bone surface with high specificity. These drug conjugate compounds may then be susceptible to cleavage by one or more proteases in the bone marrow to release and form active cytoprotective drugs (e.g., active amifostine thiol (WR1065)). This drug delivery platform may include the different components of a protective drug, a protease-cleavable peptide, and a bone-targeting ligand, as well as linkers to allow the conjugation of the drug and ligand to each terminus of the peptide. The general structure of the drug conjugate compounds as described herein can be represented by the structure shown in FIG. 1.

The protective drugs for preventing or mitigating the damage of bone marrow may be categorized as follows based on their mechanisms of action: (1) scavengers of reactive oxygen or nitrogen species (RONS) and radicals that are caused by ionizing radiation exposure; (2) neutralizers of alkylating agents and chelators to platinum drugs that are widely used in cancer chemotherapy; and (3) modulators of cell or tissue activities by activating or inhibiting specific pathways to enable cell resistance to the harmful effects of radiation or chemical compounds.

In some embodiments, the protective drugs may have the structure of formula (I) shown below:

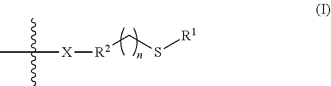

where n is an integer in the range from 0 to 16; R¹ represents H, PO₃H₂, salts of HPO₃⁻, and PO₃²⁻ anions including sodium, potassium, calcium, magnesium, or ammonium, or a compound with a disulfide bond; R² represents a linear, branched, or cyclic moiety having 1-24 carbon and/or heteroatoms and optionally having 1-6 side chains consisting of 1-18 carbon and/or heteroatoms; and X represents a cleavable chemical bond for the conjugation to the linker or peptide, i.e., an amide bond, an ester bond, a carbamate bond, a disulfide bond, a nitrogen-carbon double bond, or a nitrogen-nitrogen double bond.

In some embodiments, the protective drugs are cysteamine derivatives that may have the structure of formula (II) shown below:

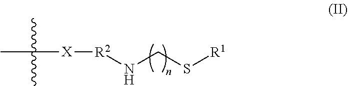

where n is an integer in the range from 2 to 3; R¹ represents H, PO₃H₂, salts of HPO₃⁻, and PO₃²⁻ anions including sodium, potassium, calcium, magnesium, or ammonium; R² represents H, or a linear, branched, or cyclic moiety having 1-24 carbon and/or heteroatoms and optionally having 1-6 side chains consisting of 1-24 carbon and/or heteroatoms; and X represents a cleavable chemical bond for the conjugation with the linker or peptide, i.e., an amide bond, an ester bond, a carbamate bond, a disulfide bond, a nitrogen-carbon double bond, or a nitrogen-nitrogen double bond. In one non-limiting embodiment, the compound is amifostine (n=2, R¹═PO₃H₂, R²═H, R³=CH₂CH₂CH₂NH₂, or the amifostine thiol, i.e., WR1065 (n=2, R¹═H, R²═H, R³═CH₂CH₂CH₂NH₂).

One embodiment described herein is a drug conjugate compound comprising: one or more bone marrow protective drug moieties; a cleavable peptide moiety having a first terminus and a second terminus; a bone-targeting ligand moiety; a first linker conjugating the bone marrow protective drug moieties to the first terminus of the cleavable peptide moiety; and a second linker conjugating the bone-targeting ligand moiety to an amino acid, where the amino acid conjugated to the bone-targeting ligand moiety is at least two amino acids away from the first terminus of the cleavable peptide moiety. In one aspect, at least one of the bone marrow protective drug moieties is a bone marrow protective drug moiety of formula (I):

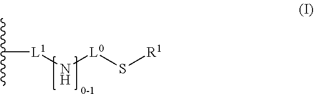

wherein:

R¹ is hydrogen or —PO₃(R^{1a})₀₋₂, wherein —PO₃(R^{1a})₀₋₂ has a net charge of 0, −1, or −2;

R^{1a}, at each occurrence, is independently hydrogen, Na⁺, K⁺, Ca⁺², Mg⁺², or NH₄⁺;

L⁰ and L¹ are each independently a C₁₋₂₄alkylene wherein optionally 1-8 methylene groups
in the C₁₋₂₄alkylene are each independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene
and/or optionally 1-4 methylene groups in the C₁₋₂₄alkylene is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the bone marrow protective drug moiety of formula (I) is a moiety of formula (I-a);

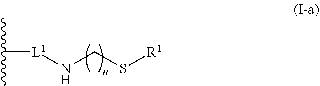

wherein n=1-24.
In another aspect, R¹ is hydrogen, —PO₃⁻², —PO₃H⁻, or —PO₃H₂.
In another aspect, L¹ comprises —N(H)-.
In another aspect, L¹ is —C₁₋₄alkylene —N(H)-.
In another aspect, n is 1-4.
In another aspect, the bone marrow protective drug moiety is

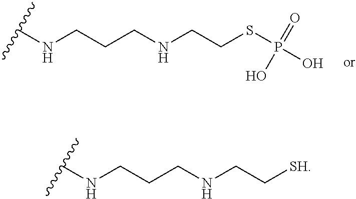 [SMILES: *NCCCNCCSP(=O)(O)O ; "or" ; *NCCCNCCS]

In another aspect, the first linker conjugates to the first terminus of the cleavable peptide moiety by a chemical bond selected from the group consisting of an amide bond, an ester bond, a sulfur-carbon single bond, a disulfide bond, a nitrogen-carbon single bond, a nitrogen-carbon double bond, and a nitrogen-nitrogen double bond.
In another aspect, the first linker connects to at least one bone marrow protective drug moiety by a linkage selected from the group consisting of

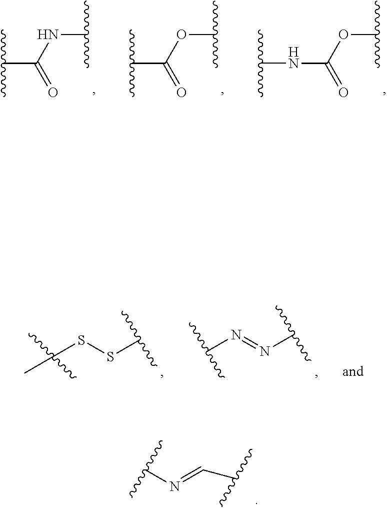

In another aspect, the first linker comprises one cleavable chemical bond between one bone marrow protective drug moiety and the first terminus of the cleavable peptide moiety.
In another aspect, the bond is an amide bond between L¹ of the bone marrow protective drug moiety and the first terminus of the cleavable peptide moiety.
In another aspect, the cleavable peptide moiety comprises less than 30 amino acids, and the amino acids are in a linear, cyclic, or branched arrangement.
In another aspect, the cleavable peptide moiety has a molecular weight of less than 3,500 Daltons.
In another aspect, the cleavable peptide moiety is a moiety of formula (II-A), (II-B), (II-C) or (II-D):

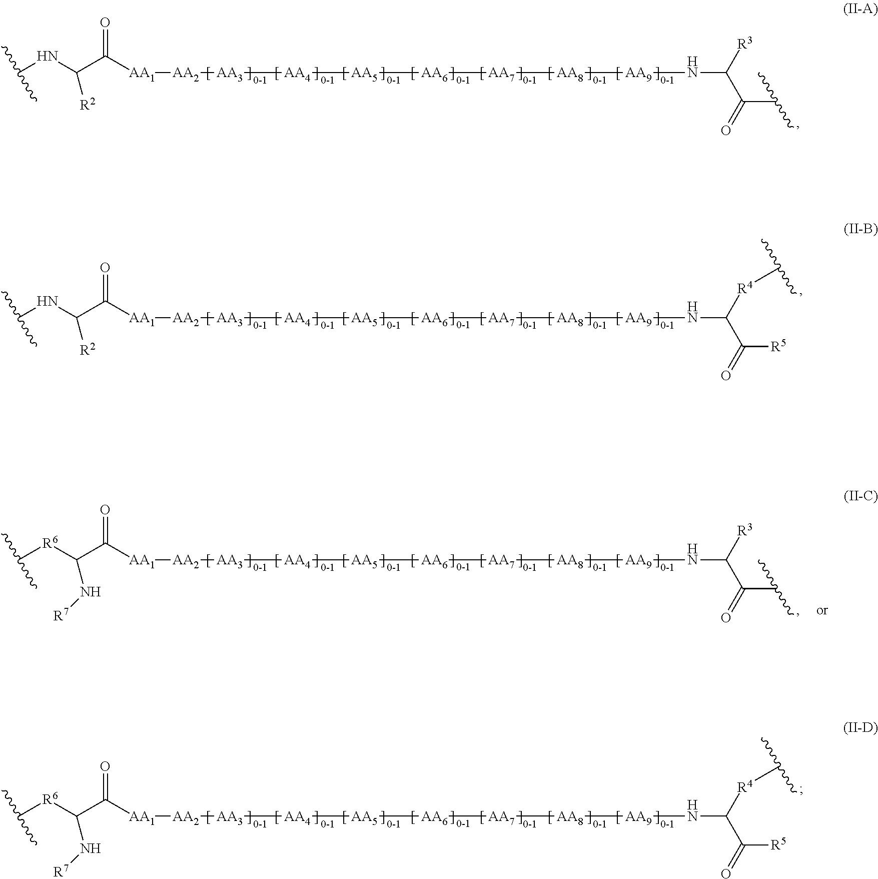

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, Y, P, Q, or V;
AA₈, at each occurance, is A, I, S, N, V, G, R, M, or Q;
AA₉, at each occurrence, is G, or S, A, L, V, W, Y, N;

R² and R³, at each occurrence, are each independently hydrogen, —CH₃,

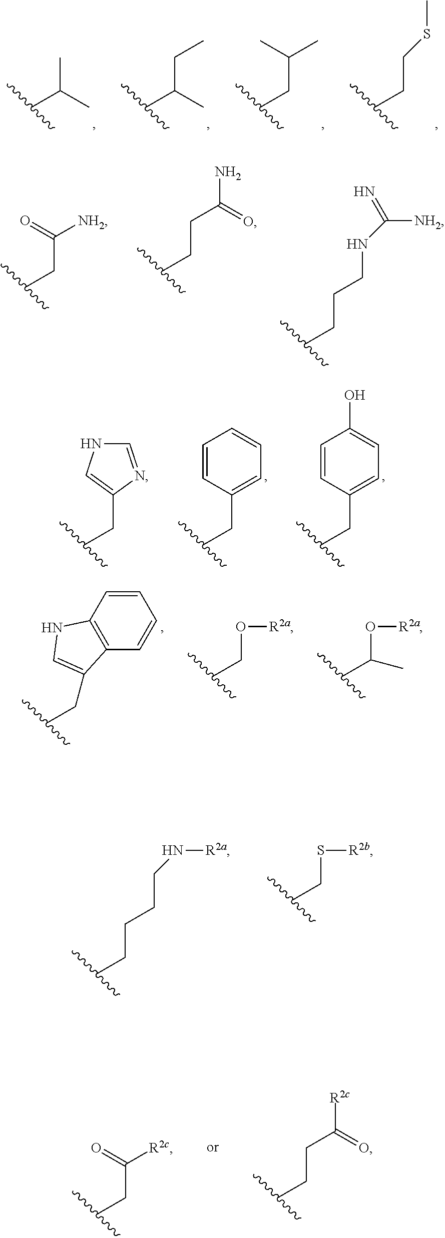

wherein:
R^{2a}, at each occurrence, is hydrogen or

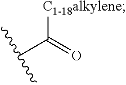

R^{2b}, at each occurrence, is hydrogen,

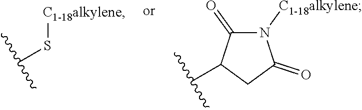

and
R^{2c}, at each occurrence, is hydrogen,

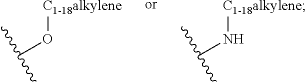

R⁴ and R⁶, at each occurrence, are each independently

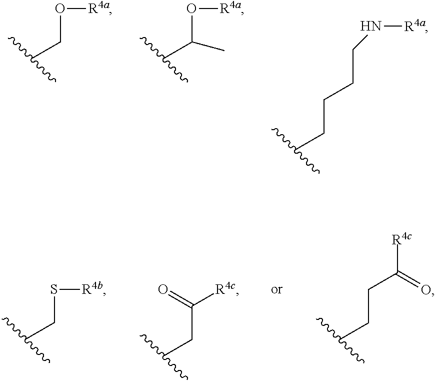

wherein:
R^{4a} is

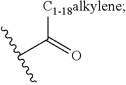

and
R^{4b} is;

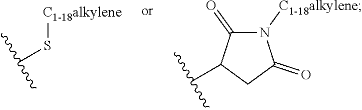

R^{4c} is

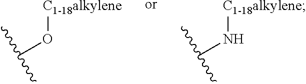

R⁵ is —OH, —O—C₁₋₁₈alkylene, or —NH—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the C₁₋₁₈alkylene is replaced with —Cy-;

R⁷ is hydrogen, or —C(O)—C₁₋₁₈alkylene, wherein optionally 1-6 methylene groups in the C₁₋₁₈alkylene are independently replaced with —N (R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the C₁₋₁₈alkylene is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the cleavable peptide moiety is a moiety of formula (II-C-A):

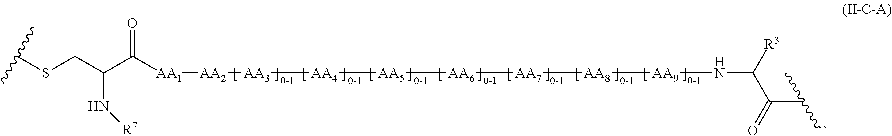

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;

R³ is hydrogen, —CH₃,

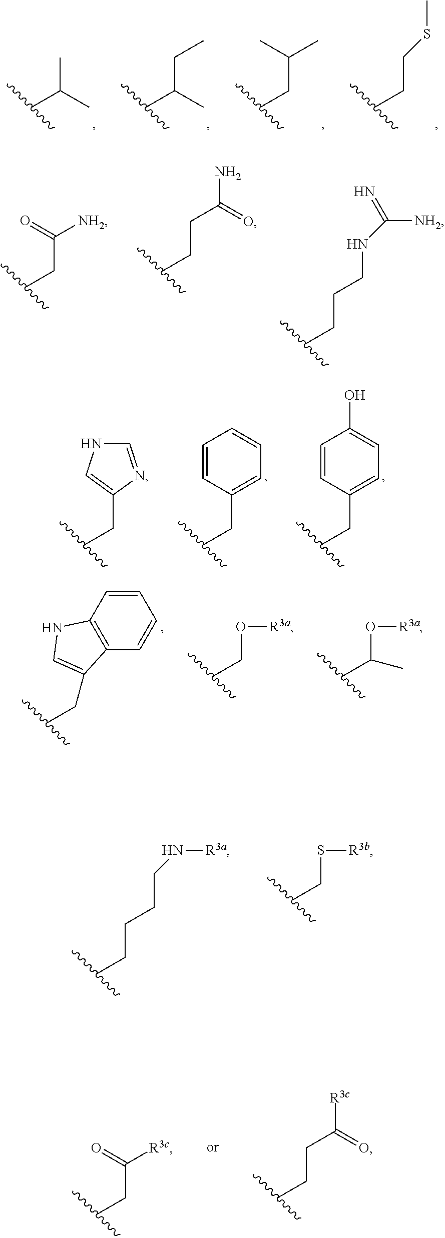

wherein:
R^{3a}, at each occurrence, is hydrogen or

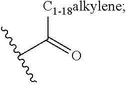

R^{3b}, at each occurrence, is hydrogen,

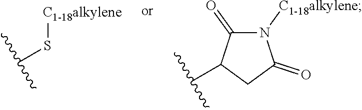

and
R^{3c}, at each occurrence, is hydrogen,

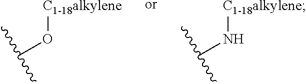

R⁷ is hydrogen, or —C(O)—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the C₁₋₁₈alkylene is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

In another aspect, the cleavable peptide moiety comprises an amino acid sequence of any one of SEQ ID NO: 1-80.
In another aspect, the cleavable peptide moiety comprises at least one sequence of PLGL (SED ID NO: 4), PIGI (SED ID NO: 5), PYSI (SED ID NO: 6), PYGI (SED ID NO: 7), PYGL (SED ID NO: 8), VLSL (SED ID NO: 9), VYGL (SED ID NO: 10), VLGL (SEQ ID NO: 11); VYSL (SED ID NO: 12), PISIY (SED ID NO: 13), PSGL (SED ID NO: 14), PLGI (SED ID NO: 15), or PMAL (SED ID NO: 16).
In another aspect, the first terminus of the cleavable peptide moiety is the carboxy (C) terminus of the cleavable peptide moiety, and the second terminus of the cleavable peptide moiety is the amino (N) terminus of the cleavable peptide moiety.
In another aspect, the cleavable peptide moiety is cleavable by one or more bone marrow-enriched proteases.
In another aspect, at least one bone marrow-enriched protease is a matrix metalloprotease, a disintegrin and metalloproteinase, a cathepsin B protease, a cathepsin K protease, a urokinase plasminogen activator protease, or a tissue-plasminogen activator protease.
In another aspect, wherein the bone-targeting ligand moiety is a bisphosphonate moiety of formula (IV):

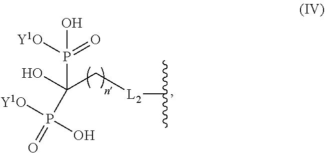

wherein:

Y¹, at each occurrence, is independently hydrogen, Na⁺, K⁺, Ca⁺², Mg⁺², or NH₄⁺;

n′is 1-16;

L² is a C₁₋₂₄alkylene wherein optionally 1-8 methylene groups in the alkylene of L² are independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of L² is replaced with-Cy-;

Cy, at each occurrence, is C₃₋₆ cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

RX, at each occurrence, is independently hydrogen or C₁₋₄alkyl.

In another aspect, Y¹, at each occurrence, is independently hydrogen, Na⁺, K⁺, Ca⁺², Mg⁺², or NH₄⁺.
In another aspect, n′is 0-4.
In another aspect, L² comprises —N(H)—, —N(H)—C₁₋₄alkylene-, —N(CH₃)—C₁₋₄alkylene-, phenylene, imidazolylene, or triazolylene.
In another aspect, the bone-targeting ligand moiety is

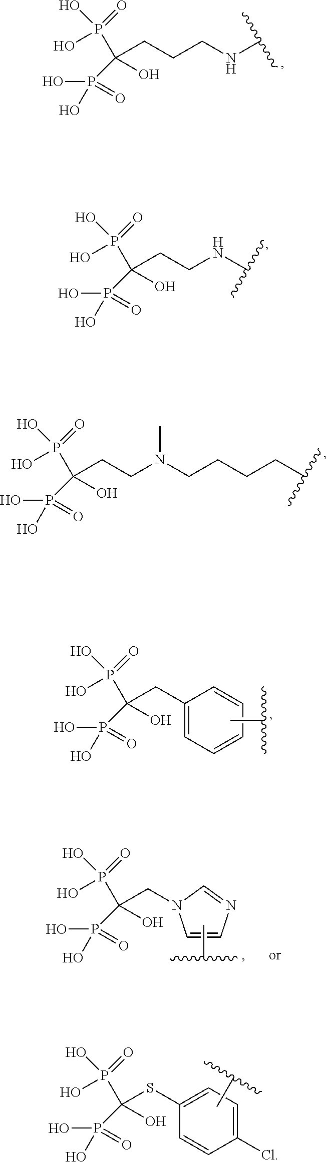

In another aspect, the second linker connects to bisphosphonate moiety by a linkage selected from the group consisting of

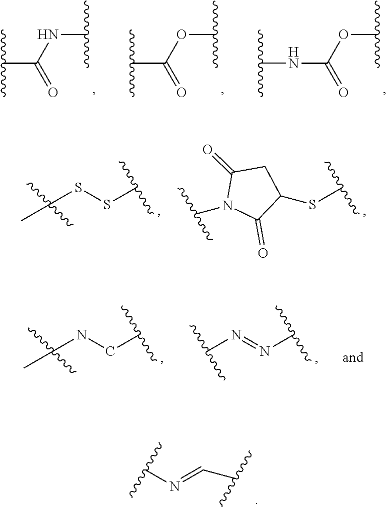

In another aspect, the second linker is a —C₁₋₂₄alkylene-wherein optionally 1-8 methylene groups in the second linker are
independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of the second linker is replaced with —Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
In another aspect, the second linker comprises a maleimide moiety, wherein the maleimide moiety is attached to a cysteine moiety that is at least two amino acids away from the first terminus of the cleavable peptide moiety.
In another aspect, the second linker is a linker having formula:

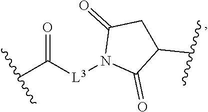

wherein:

L³ is a C₁₋₁₈alkylene wherein optionally 1-6 methylene groups in the alkylene of L³ are independently replaced with —N(R^{X})—, —O—, —S—, —SO—, —SO₂-, or —C(O)-, wherein 2 methylene groups replaced with —N(R_{X})—, —O—, —S—, —SO—, or —SO₂- are separated by two or more carbon atoms in the alkylene and/or optionally 1-4 methylene groups in the alkylene of L³ is replaced with —Cy-;

Cy, at each occurrence, is a C₃₋₆cycloalkylene, a phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.

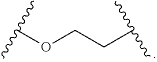 [SMILES: *CCO*]

In another aspect, L³ comprises repeating units of
In another aspect, the second linker is:

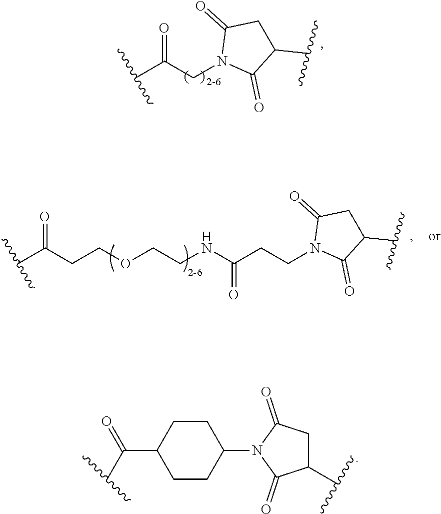

Another embodiment described herein is a pharmaceutical composition comprising one or more of the drug conjugate compounds described herein and a pharmaceutically acceptable carrier.

The protease-cleavable peptides may have distinct amino acid sequences to allow cleavage of the drug conjugates by bone marrow-enriched proteases, and thus mediate the release of drug-containing moieties (with residual amino acids conjugated to the drug molecule) from the bone surface to the bone marrow. Furthermore, the released drug-containing moieties may be converted into the parent drug by bone marrow-enriched proteases with the removal of residual amino acids conjugated to the drug molecules. In one non-limiting embodiment, the bone marrow-enriched proteases include the matrix metalloproteases (MMPs), the disintegrin and metalloproteinases, cathepsin B, cathepsin K, the urokinase plasminogen activator, and the tissue-plasminogen activator. The protease-cleavable peptides as described herein may have a length of less than 30 amino acids and a total molecular weight of less than 3.5 kDa. The amino acid sequences including protease cleavage sites may be represented as follows using standard single-letter amino acids: -R-R-, -F-R-, -I-P-K-, K-K-, -R-K-, -L-R-, -V-R-, -A-F-L-, -S-S-Y-Y-S-R-(SEQ ID NO: 1), -S-S-Y-Y-S-L-(SEQ ID NO: 2), -R-S-S-Y-Y-S-L-(SEQ ID NO: 3), -P-L-G-L-(SEQ ID NO: 4), -P-I-G-I-(SEQ ID NO: 5), -P-Y-S-I-(SEQ ID NO: 6), -P-Y-G-I-(SEQ ID NO: 7), P-Y-G-L-(SEQ ID NO: 8), -V-L-S-L-(SEQ ID NO: 8), -V-Y-G-L-(SEQ ID NO: 10), -V-L-G-L-(SEQ ID NO: 11), -V-Y-S-L-(SEQ ID NO: 12), -P-I-S-I-Y-(SEQ ID NO: 13), -P-S-G-L-(SEQ ID NO: 14), -P-L-G-I-(SEQ ID NO: 15), -P-M-A-L-(SEQ ID NO: 16), -F-P-K-F-F-S-Q-(SEQ ID NO: 17), -G-F-L-G-(SEQ ID NO: 18), -K-P-I-E-F-(SEQ ID NO: 19), -G-P-L-G-I-A-G-Q-(SEQ ID NO: 20), -G-P-E-G-I-W-Q-(SEQ ID NO: 21), -G-G-,-G-G-G-, and -G-G-G-R-R-(SEQ ID NO: 22). In some embodiments, the protease-cleavable peptides may include an acetyl moiety on the N-terminus.

In some embodiments, the specific amino acid sequence motifs of -P-L-G-L-(SEQ ID NO: 4), -P-I-G-I-(SEQ ID NO: 5), -P-Y-S-I-(SEQ ID NO: 6), -P-Y-G-I-(SEQ ID NO: 7), P-Y-G-L-(SEQ ID NO: 8), -V-L-S-L-(SEQ ID NO: 8), -V-Y-G-L-(SEQ ID NO: 10), -V-L-G-L-(SEQ ID NO: 11), -V-Y-S-L-(SEQ ID NO: 12), -P-I-S-I-Y-(SEQ ID NO: 13), -P-S-G-L-(SEQ ID NO: 14), -P-L-G-I-(SEQ ID NO: 15), -P-M-A-L-(SEQ ID NO: 16) may be cleavable by a matrix metalloprotease (e.g., MMP9). In some embodiments, the peptides (with or without N-terminal acetylation) that are cleavable by matrix metalloproteases may include APIGIAL (SEQ ID NO: 23), APLGLYAL (SEQ ID NO: 24), CKPISIAL (SEQ ID NO: 25), CVLSLYAL (SEQ ID NO: 26), VYGLIAL (SEQ ID NO: 27), GPLSLSAL (SEQ ID NO: 28), CPLGLYAL (SEQ ID NO: 29), CPISIUVI (SEQ ID NO: 30), CQPSGLAL (SEQ ID NO: 31), GPYSIYAL (SEQ ID NO: 32), CKPLGLWAL (SEQ ID NO: 33), CRPMALIIAL (SEQ ID NO: 34), CQPLGALYAL (SEQ ID NO: 35), CKPSGLSLAGI (SEQ ID NO: 36), APSGLYALISL (SEQ ID NO: 37), CQPLGLYAG (SEQ ID NO: 38), CRPLGLWAL (SEQ ID NO: 39), CPISIALGAL (SEQ ID NO: 40), CPYSIYALGL (SEQ ID NO: 41), CKVLGLIAL (SEQ ID NO: 42), CGQVYGLAII (SEQ ID NO: 43), or CRVLGLIAL (SEQ ID NO: 44).

In some embodiments, the peptides (with or without acetylation at the N-terminus) that are cleavable by cathepsin B or cathepsin K may include CFRL (SEQ ID NO: 45), ALRG (SEQ ID NO: 46), CVRGG (SEQ ID NO: 47), CALRGAI (SEQ ID NO: 48), IGFLGIGL (SEQ ID NO: 49), AIGFLGAV (SEQ ID NO: 50), CPGFLGIA (SEQ ID NO: 51), CPFGFLGIAL (SEQ ID NO: 52), CRPGFLGAL (SEQ ID NO: 53), IAGFRGIAK (SEQ ID NO: 54), PGFRKIAG (SEQ ID NO: 55), CPVRIAKG (SEQ ID NO: 56), CQPVRIAL (SEQ ID NO: 57), CQFRVIAL (SEQ ID NO: 58), CKPFRVGAL (SEQ ID NO: 59), CRPAFLGVL (SEQ ID NO: 60), CQPAFLGVL (SEQ ID NO: 61), or CIGVRGIAQL (SEQ ID NO: 62).

As used herein, the term “ligand” refers to a molecular species or compound having binding affinity to a binding target. Described herein are “bone-targeting ligands” that may target the bone turnover surface, for bone marrow-specific delivery of protective drugs.

In some embodiments, oligopeptides are used as bone-targeting ligands for bone turnover surface targeting. The bone turnover surface is constituted of highly crystallized hydroxyapatite and proteins that are involved in the elaborate regulation of osteoclast binding and bone mineralization. The bone-specific targeting of oligopeptides is achieved by (1) the ionic interaction with positively-charged calcium ions in the highly crystallized hydroxyapatite (Murphy et al., Biomacromolecules, 8 (7): 2237-2243 (2007)); or (2) special proteins existing on the bone surface such as casein kinase 2-interacting protein-1 (Zhang et al., Nature Med. 18 (2): 307-314 (2012)).

Oligopeptides described herein may have the following amino acid sequences using standard single-letter amino acids: —(E)ₙ-, where n is an integer in the range from 4 to 16;-(D)ₙ-, where n is an integer in the range from 4 to 16; or —(D—S—S)ₙ-, where n is an integer in the range from 1 to 6.

In some embodiments, bisphosphonates are ligands for bone marrow-specific delivery (i.e., bone-targeting ligands). Bisphosphonates are comprised of a unique phosphorus-carbon-phosphorus backbone and have been previously studied as ligands that specifically direct drugs to the bone turnover area, which directly contacts bone marrow. Russell, Bone, 49 (1): 2-19 (2011). The binding of the bisphosphonate-containing conjugates to bone is achieved via the strong coordination between the bisphosphonate structure and the calcium ions on the surface of hydroxyapatite, which is the main component of the subject bone matrix. Described herein are bisphosphonates that may have the structure of formula (III) as shown below:

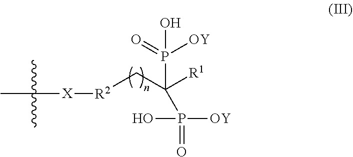

where n is an integer in the range from 0 to 12; R¹ represents a linear, branched, or cyclic moiety having 1-12 carbons and/or heteroatoms; R² represents C, S, O, NH, CO, a triazole moiety, or an imidazole moiety; X represents a chemical bond between R² and a linker, i.e., an amide bond, an ester bond, a sulfur-carbon single bond, a disulfide bond, a nitrogen-carbon single bond, a nitrogen-carbon double bond, a carbon-carbon single bond, a carbon-carbon double bond, or a carbon-carbon triple bond; and Y is independently selected from hydrogen, sodium, potassium, calcium, magnesium, or ammonium. In some embodiments, the bone-targeting ligands may be bisphosphonates, including Alendronate, Pamidronate, Ibandronate, Risedronate, Zoledronate, and Tiludronate, the structures of which are shown below:

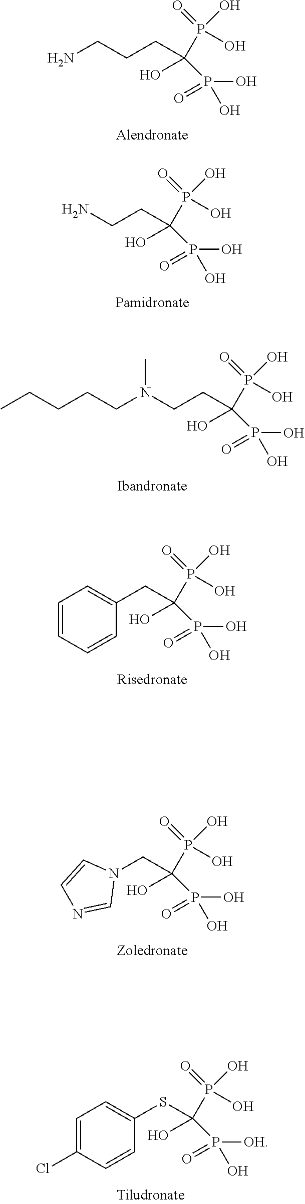 [SMILES: NCCCC(O)(P(=O)(O)O)P(=O)(O)O ; NCCC(O)(P(=O)(O)O)P(=O)(O)O ; CCCCCN(C)CCC(O)(P(=O)(O)O)P(=O)(O)O ; O=P(O)(O)C(O)(Cc1ccccc1)P(=O)(O)O ; O=P(O)(O)C(O)(Cn1ccnc1)P(=O)(O)O ; O=P(O)(O)C(O)(Sc1ccc(Cl)cc1)P(=O)(O)O]

In some embodiments, one or more linkers are used to conjugate the different components of the described bone marrow-specific protective drug conjugates. For example, a first linker (i.e., “Linker 1”) may be used to conjugate a protective drug to one terminus of a protease-cleavable peptide, while a second independent linker (i.e., “Linker 2”) may be used to conjugate a bone-targeting ligand to the other terminus of the protease-cleavable peptide. In some embodiments, Linker 1 and Linker 2 may comprise one or more chemical bonds. In some embodiments, Linker 1 and Linker 2 may be the same. In some embodiments, Linker 1 and Linker 2 may be different.

In some embodiments, Linker 1 allows the conjugation of the protective drug to a first terminus of the peptide. In some embodiments, Linker 1 represents a cleavable chemical bond (i.e., an amide bond, an ester bond, a carbamate bond, a disulfide bond, a nitrogen-carbon double bond, or a nitrogen-nitrogen double bond) when the protective drug is conjugated to the peptide directly.

In some embodiments, Linker 1 may have the structure shown below to conjugate a protective drug to a terminus of a protease-cleavable peptide:

### Linker 1

### Protective Drug —X—R—Y- Peptide

where X represents a cleavable chemical bond conjugating the linker to the protective drug, i.e., an amide bond, an ester bond, a carbamate bond, a disulfide bond, a nitrogen-carbon double bond, or a nitrogen-nitrogen double bond; R represents a linear, branched, or cyclic moiety having 0-128 carbon and/or heteroatoms; and Y represents a chemical bond for the conjugation of the linker to the peptide, i.e., an amide bond, an ester bond, a diazo bond, a sulfur-carbon single bond, a disulfide bond, a nitrogen-carbon single bond, a nitrogen-carbon double bond, a nitrogen-nitrogen double bond, a carbon-carbon single bond, a carbon-carbon double bond, or a carbon-carbon triple bond.

In some embodiments, Linker 2 allows the conjugation of the ligand (e.g., bone-targeting ligand) to a second terminus of the peptide. In some embodiments, Linker 2 represents a chemical bond (i.e., an amide bond, an ester bond, a diazo bond, a sulfur-carbon single bond, a disulfide bond, a nitrogen-carbon single bond, a nitrogen-carbon double bond, a nitrogen-nitrogen double bond, a carbon-carbon single bond, a carbon-carbon double bond, or a carbon-carbon triple bond) when the ligand is conjugated to the peptide directly.

In some embodiments, Linker 2 may have the structure shown below to conjugate a ligand to a terminus of a protease-cleavable peptide:

### Linker 2

### Peptide —X—R—Y- Ligand

where X represents a chemical bond for the conjugation of the linker to the peptide, i.e., an amide bond, an ester bond, a carbon oxygen bond, a sulfur-carbon single bond, a disulfide bond, a nitrogen-carbon single bond, a nitrogen-carbon double bond, a nitrogen-nitrogen double bond, a carbon-carbon single bond, a carbon-carbon double bond, or a carbon-carbon triple bond; R represents a linear, branched, or cyclic moiety having 0-128 carbon and/or heteroatoms; and Y represents a chemical bond for the conjugation of the linker to the ligand, i.e., an amide bond, an ester bond, a carbon oxygen bond, a sulfur-carbon single bond, a disulfide bond, a nitrogen-carbon single bond, a nitrogen-carbon double bond, a nitrogen-nitrogen double bond, a carbon-carbon single bond, a carbon-carbon double bond, or a carbon-carbon triple bond.

In some embodiments, Linker 2 may conjugate a peptide to a ligand with the structure shown below:

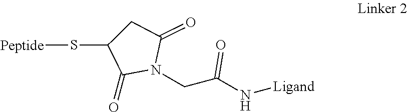

where R represents a linear, branched, or cyclic moiety having 0-128 carbon and/or heteroatoms. Linker 2 may have a thiosuccinimide group, which is the conjugation product from a thiol group in the peptide and a maleimide attached to R, and an amide bond formed between the carboxyl acid group of R and an amine group in the ligand. In certain non-limiting embodiments, Linker 2 may be maleimide-PEG₂-NHS, maleimide-PEG₆-NHS, maleimide-C₆-NHS, or maleimide-cyclohexanoic acid-NHS (SMCC), the structures of which are shown below:

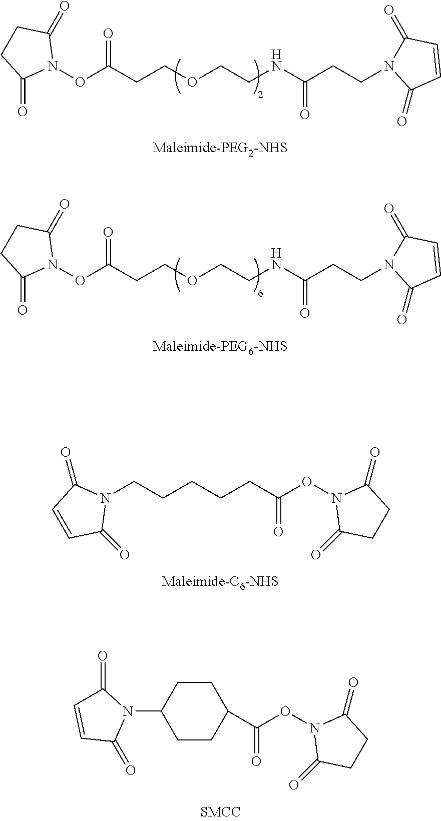

The general synthesis pathway of the described bone marrow protective drug conjugates is not restricted. One non-limiting example is described as follows: (1) peptides are pretreated to introduce protective groups into the molecules; (2) the protective drug molecules are directly conjugated to the ends of the pre-treated peptides by forming cleavable chemical bonds; and (3) the resultant conjugates are deprotected, followed by coupling with functionalized bisphosphonates to form a bone marrow-specific protective drug conjugate.

Provided below are non-limiting structures of various exemplary bone marrow-specific protective drug conjugates having the indicated peptide sequences:

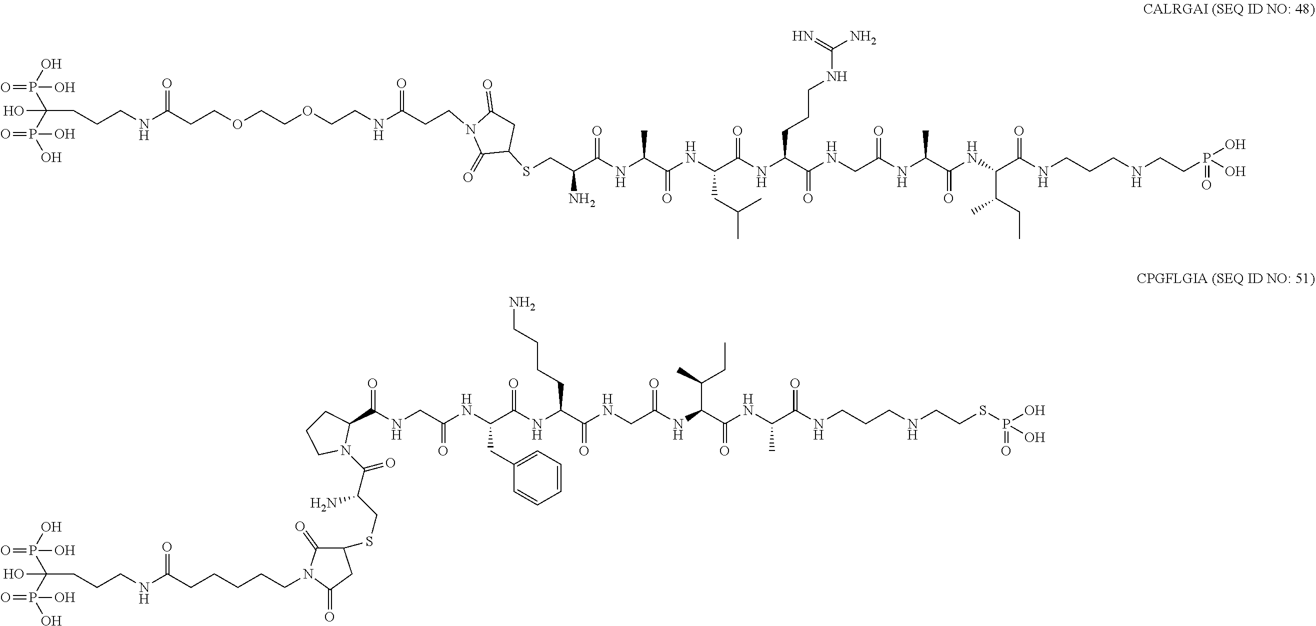 [SMILES: CC[C@H](C)[C@H](NC(=O)[C@H](C)NC(=O)CNC(=O)[C@H](CCCNC(=N)N)NC(=O)[C@H](CC(C)C)NC(=O)[C@H](C)NC(=O)[C@@H](N)CSC1CC(=O)N(CCC(=O)NCCOCCOCCC(=O)NCCCC(O)(P(=O)(O)O)P(=O)(O)O)C1=O)C(=O)NCCCNCCP(=O)(O)O ; CC[C@H](C)[C@H](NC(=O)CNC(=O)[C@H](CCCCN)NC(=O)[C@H](Cc1ccccc1)NC(=O)CNC(=O)[C@@H]1CCCN1C(=O)[C@@H](N)CSC1CC(=O)N(CCCCCC(=O)NCCCC(O)(P(=O)(O)O)P(=O)(O)O)C1=O)C(=O)N[C@@H](C)C(=O)NCCCNCCSP(=O)(O)O]

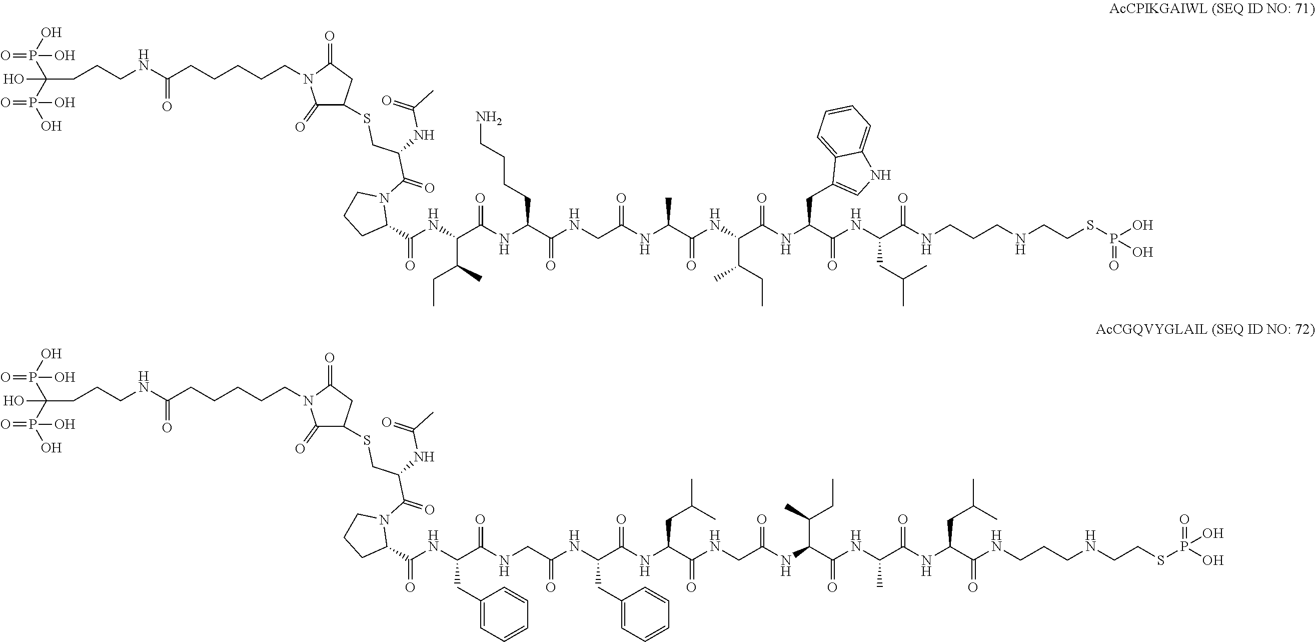 [SMILES: CC[C@H](C)[C@H](NC(=O)[C@H](C)NC(=O)CNC(=O)[C@H](CCCCN)NC(=O)[C@@H](NC(=O)[C@@H]1CCCN1C(=O)[C@H](CSC1CC(=O)N(CCCCCC(=O)NCCCC(O)(P(=O)(O)O)P(=O)(O)O)C1=O)NC(C)=O)[C@@H](C)CC)C(=O)N[C@@H](Cc1c[nH]c2ccccc12)C(=O)N[C@@H](CC(C)C)C(=O)NCCCNCCSP(=O)(O)O ; CC[C@H](C)[C@H](NC(=O)CNC(=O)[C@H](CC(C)C)NC(=O)[C@H](Cc1ccccc1)NC(=O)CNC(=O)[C@H](Cc1ccccc1)NC(=O)[C@@H]1CCCN1C(=O)[C@H](CSC1CC(=O)N(CCCCCC(=O)NCCCC(O)(P(=O)(O)O)P(=O)(O)O)C1=O)NC(C)=O)C(=O)N[C@@H](C)C(=O)N[C@@H](CC(C)C)C(=O)NCCCNCCSP(=O)(O)O]

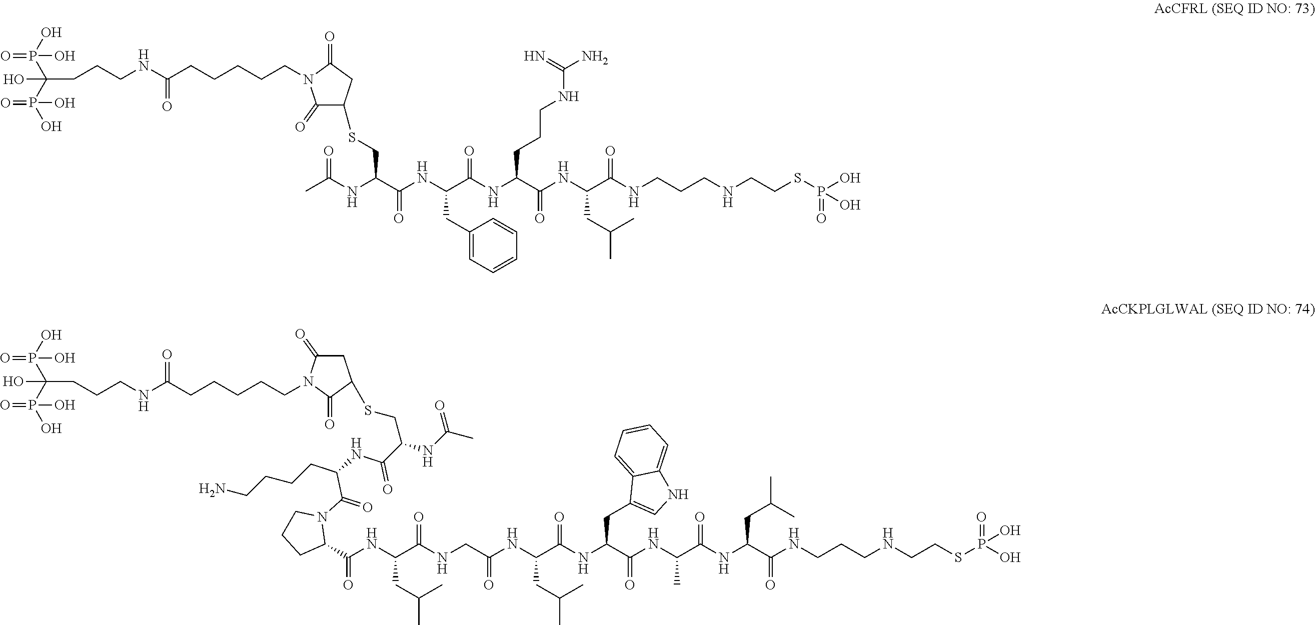 [SMILES: CC(=O)N[C@@H](CSC1CC(=O)N(CCCCCC(=O)NCCCC(O)(P(=O)(O)O)P(=O)(O)O)C1=O)C(=O)N[C@@H](Cc1ccccc1)C(=O)N[C@@H](CCCNC(=N)N)C(=O)N[C@@H](CC(C)C)C(=O)NCCCNCCSP(=O)(O)O ; CC(=O)N[C@@H](CSC1CC(=O)N(CCCCCC(=O)NCCCC(O)(P(=O)(O)O)P(=O)(O)O)C1=O)C(=O)N[C@@H](CCCCN)C(=O)N1CCC[C@H]1C(=O)N[C@@H](CC(C)C)C(=O)NCC(=O)N[C@@H](CC(C)C)C(=O)N[C@@H](Cc1c[nH]c2ccccc12)C(=O)N[C@@H](C)C(=O)N[C@@H](CC(C)C)C(=O)NCCCNCCSP(=O)(O)O]

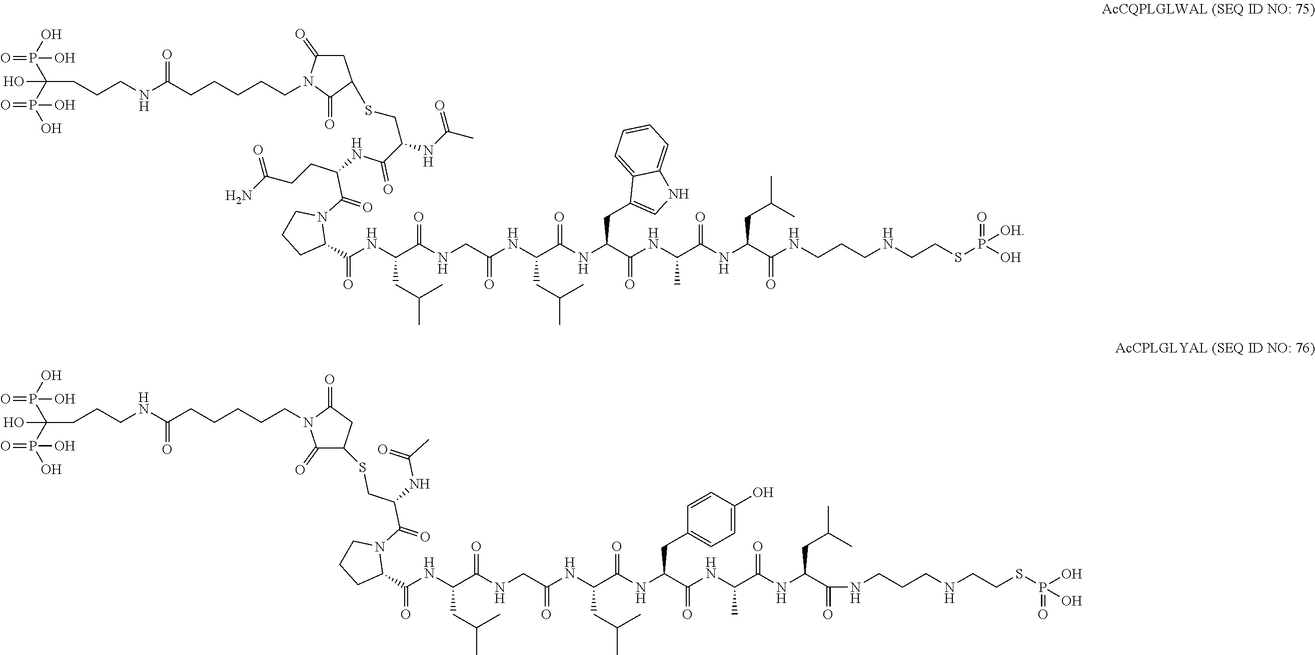 [SMILES: CC(=O)N[C@@H](CSC1CC(=O)N(CCCCCC(=O)NCCCC(O)(P(=O)(O)O)P(=O)(O)O)C1=O)C(=O)N[C@@H](CCC(N)=O)C(=O)N1CCC[C@H]1C(=O)N[C@@H](CC(C)C)C(=O)NCC(=O)N[C@@H](CC(C)C)C(=O)N[C@@H](Cc1c[nH]c2ccccc12)C(=O)N[C@@H](C)C(=O)N[C@@H](CC(C)C)C(=O)NCCCNCCSP(=O)(O)O ; CC(=O)N[C@@H](CSC1CC(=O)N(CCCCCC(=O)NCCCC(O)(P(=O)(O)O)P(=O)(O)O)C1=O)C(=O)N1CCC[C@H]1C(=O)N[C@@H](CC(C)C)C(=O)NCC(=O)N[C@@H](CC(C)C)C(=O)N[C@@H](Cc1ccc(O)cc1)C(=O)N[C@@H](C)C(=O)N[C@@H](CC(C)C)C(=O)NCCCNCCSP(=O)(O)O]

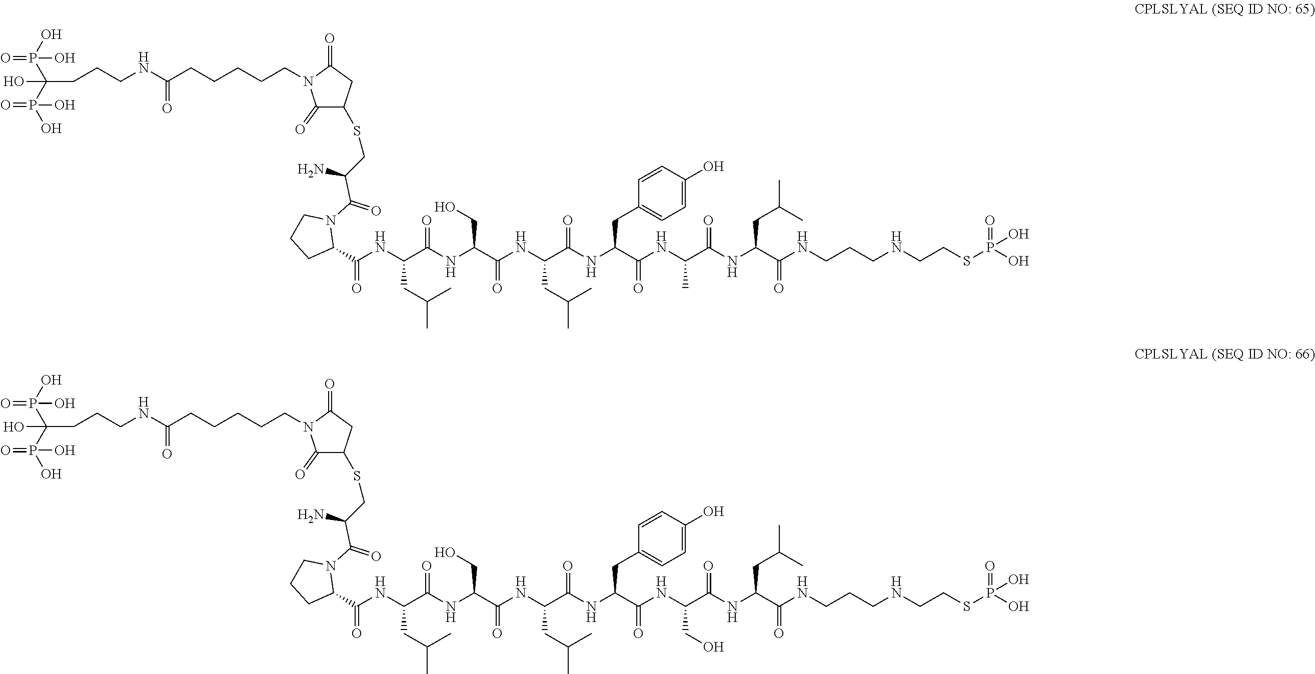 [SMILES: CC(C)C[C@H](NC(=O)[C@H](C)NC(=O)[C@H](Cc1ccc(O)cc1)NC(=O)[C@H](CC(C)C)NC(=O)[C@H](CO)NC(=O)[C@H](CC(C)C)NC(=O)[C@@H]1CCCN1C(=O)[C@@H](N)CSC1CC(=O)N(CCCCCC(=O)NCCCC(O)(P(=O)(O)O)P(=O)(O)O)C1=O)C(=O)NCCCNCCSP(=O)(O)O ; CC(C)C[C@H](NC(=O)[C@H](CO)NC(=O)[C@H](Cc1ccc(O)cc1)NC(=O)[C@H](CC(C)C)NC(=O)[C@H](CO)NC(=O)[C@H](CC(C)C)NC(=O)[C@@H]1CCCN1C(=O)[C@@H](N)CSC1CC(=O)N(CCCCCC(=O)NCCCC(O)(P(=O)(O)O)P(=O)(O)O)C1=O)C(=O)NCCCNCCSP(=O)(O)O]

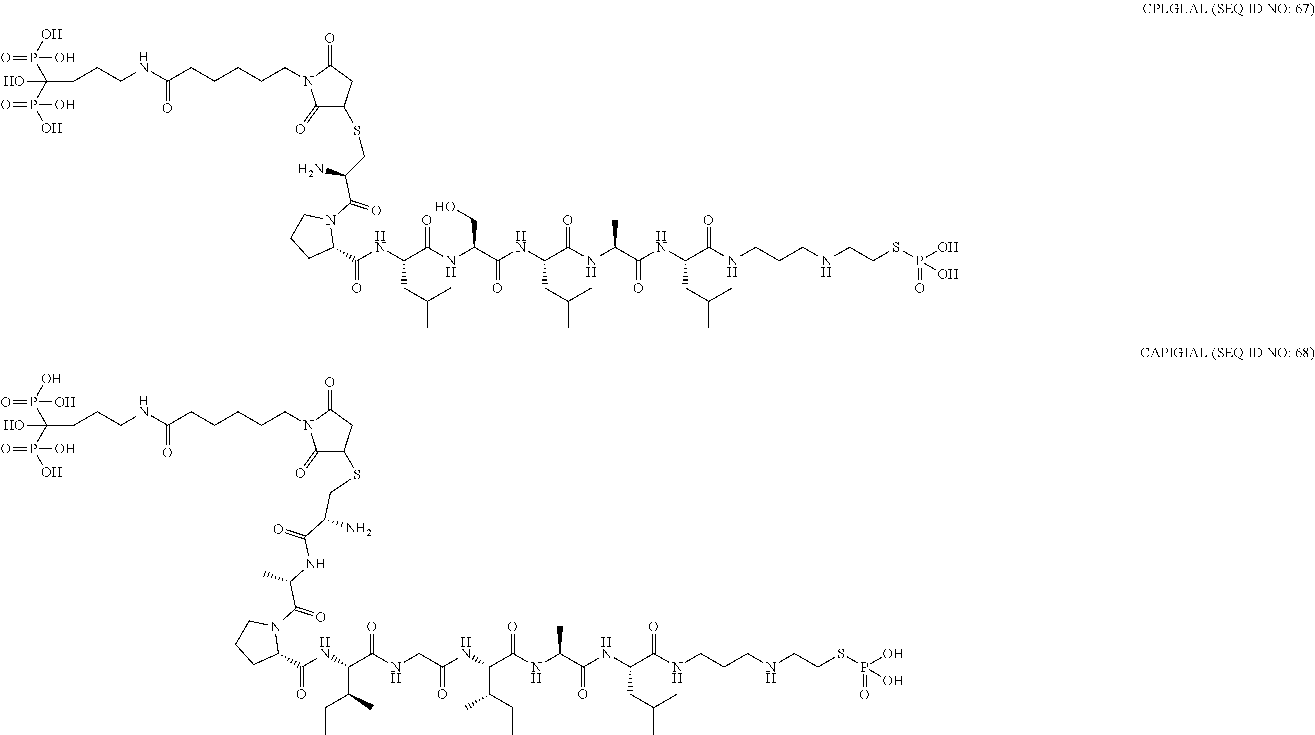 [SMILES: CC(C)C[C@H](NC(=O)[C@H](C)NC(=O)[C@H](CC(C)C)NC(=O)[C@H](CO)NC(=O)[C@H](CC(C)C)NC(=O)[C@@H]1CCCN1C(=O)[C@@H](N)CSC1CC(=O)N(CCCCCC(=O)NCCCC(O)(P(=O)(O)O)P(=O)(O)O)C1=O)C(=O)NCCCNCCSP(=O)(O)O ; CC[C@H](C)[C@H](NC(=O)CNC(=O)[C@@H](NC(=O)[C@@H]1CCCN1C(=O)[C@H](C)NC(=O)[C@@H](N)CSC1CC(=O)N(CCCCCC(=O)NCCCC(O)(P(=O)(O)O)P(=O)(O)O)C1=O)[C@@H](C)CC)C(=O)N[C@@H](C)C(=O)N[C@@H](CC(C)C)C(=O)NCCCNCCSP(=O)(O)O]

### Compositions and Methods

Another embodiment described herein relates to a pharmaceutical composition comprising the conjugates of the protective drugs as defined above, and optionally a pharmaceutically acceptable carrier and/or a pharmaceutically acceptable adjuvant and/or a diluent. In some embodiments, these drug conjugate compositions may be used in methods of targeted delivery of a drug to bone marrow in a subject or in methods of protecting a subject from radiation or chemotherapeutic toxicity.

The pharmaceutical composition may, for example, contain solvents and diluents such as a solution containing any pharmaceutically acceptable inactive ingredients. In one example, the solution is a saline solution containing 5% (volume/volume) of PEG300. Moreover, the pharmaceutical compositions described herein may be in any form suitable for administration to a patient, for example in an injectable form, as a tablet or a capsule, or as a composition for inhalation.

According to one non-limiting embodiment, the above-defined pharmaceutical composition is used for the prevention of bone marrow diseases, especially for the HSPCs in multiple diseases, including ionizing radiation-induced acute hematopoietic subsyndrome, chemotherapy-caused myelosuppression, and radiotherapy-induced myelotoxicity.

In some embodiments, the bone marrow protective drug as defined above may be comprised in a kit, which may further contain one or more adjuvants, such as a buffer, excipient, or a pharmaceutically acceptable carrier.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

Various embodiments and aspects of the inventions described herein are summarized by the following clauses:
Clause 1. A drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.
Clause 2. The drug conjugate compound of clause 1, wherein at least one of the bone marrow protective drug moieties is a bone marrow protective drug moiety of formula (I):

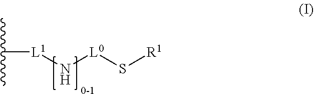

wherein:

R¹ is hydrogen or —PO₃(R^{1a})₀₋₂, wherein —PO₃(R^{1a})₀₋₂ has a net charge of 0, −1, or −2;

R^{1a}, at each occurrence, is independently hydrogen, Na⁺, K⁺, Ca⁺², Mg⁺², or NH₄⁺;

L⁰ and L¹ are each independently a C₁₋₂₄alkylene wherein optionally 1-8 methylene groups in the C₁₋₂₄alkylene are each independently replaced with —N(R^{X})—, —O—, —S-,

—SO—, —SO₂-, or —C(O)-, wherein 2 methylene groups replaced with —N(R^{X})-,

—O—, —S—, —SO-, or —SO₂- are separated by two or more carbon atoms in the alkylene and/or optionally 1-4 methylene groups in the C₁₋₂₄alkylene is replaced with —Cy-;

Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
Clause 3. The drug conjugate compound of clause 2, wherein the bone marrow protective drug moiety of formula (I) is a moiety of formula (I-a):

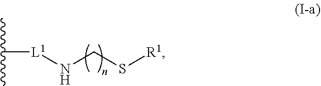

wherein n=1-24.
Clause 4. The drug conjugate compound of clause 2 or 3, wherein R¹ is hydrogen, —PO₃⁻², —PO₃H⁻, or —PO₃H₂.
Clause 5. The drug conjugate compound of any one of clauses 2-4, wherein L¹ comprises —N(H)-.
Clause 6. The drug conjugate compound of any one of clauses 2-5, wherein L¹ is —C₁₋₄alkylene-N (H)-.
Clause 7. The drug conjugate compound of any one of clauses 2-6, wherein n is 1-4.
Clause 8. The drug conjugate compound of any one of clauses 2-7, wherein the bone marrow protective drug moiety is

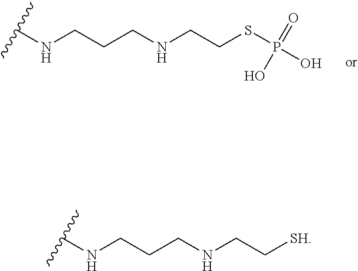 [SMILES: *NCCCNCCSP(=O)(O)O ; "or" ; *NCCCNCCS]

Clause 9. The drug conjugate compound of any one of clauses 1-8, wherein the first linker conjugates to the first terminus of the cleavable peptide moiety by a chemical bond selected from the group consisting of an amide bond, an ester bond, a sulfur-carbon single bond, a disulfide bond, a nitrogen-carbon single bond, a nitrogen-carbon double bond, and a nitrogen-nitrogen double bond.
Clause 10. The drug conjugate compound of any one of clauses 1-9, wherein the first linker connects to at least one bone marrow protective drug moiety by a linkage selected from the group consisting of

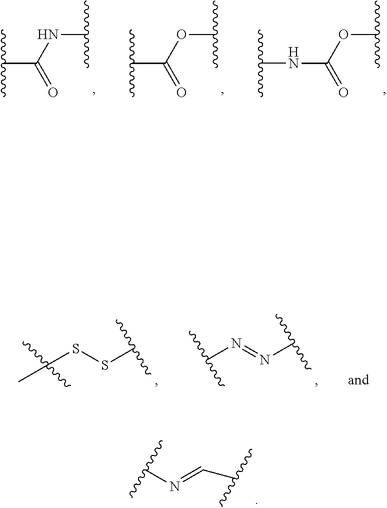

Clause 11. The drug conjugate compound of any one of clauses 1-10, wherein the first linker comprises one cleavable chemical bond between one bone marrow protective drug moiety and the first terminus of the cleavable peptide moiety.
Clause 12. The drug conjugate compound of clause 11, wherein the bond is an amide bond between L¹ of the bone marrow protective drug moiety and the first terminus of the cleavable peptide moiety.
Clause 13. The drug conjugate compound of any one of clauses 1-12, wherein the cleavable peptide moiety comprises less than 30 amino acids, and the amino acids are in a linear, cyclic, or branched arrangement.
Clause 14. The drug conjugate compound of any one of clauses 1-13, wherein the cleavable peptide moiety has a molecular weight of less than 3,500 Daltons.
Clause 15. The drug conjugate compound of any one of clauses 1-14, wherein the cleavable peptide moiety is a moiety of formula (II-A), (II-B), (II-C) or (II-D):

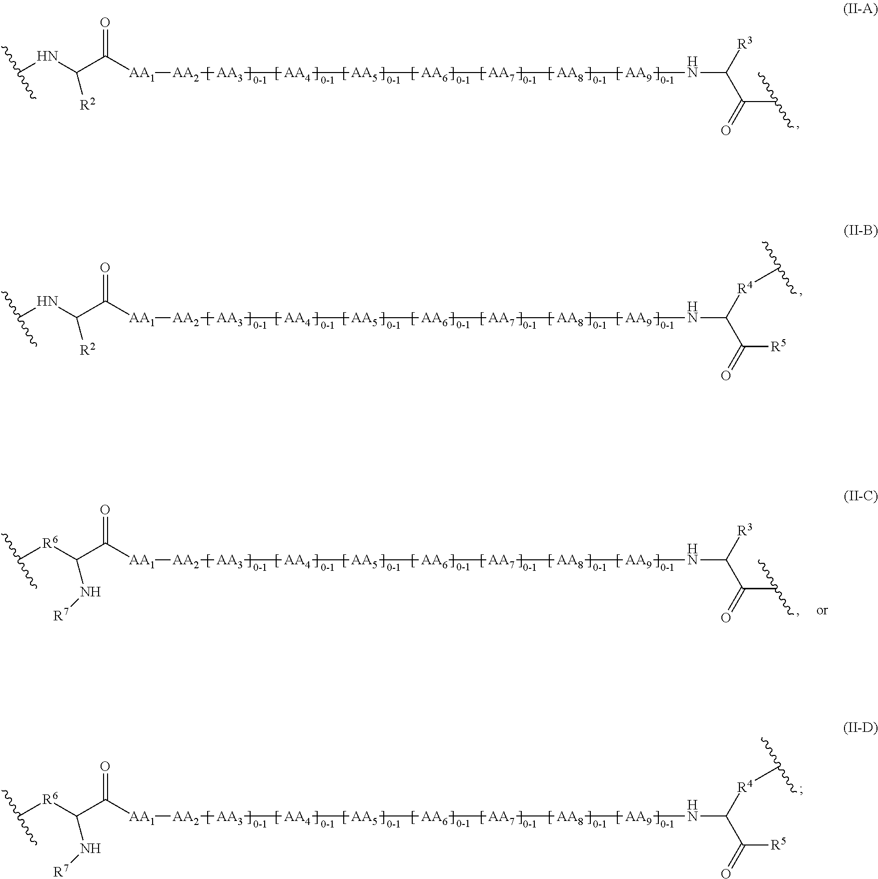

wherein:

AA₁, AA₂, AA₃, AA₄, AA₅, AA₆, AA₇, AA₈, and AA₉, are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, Y, P, Q, or V;
AA₈, at each occurrence, is A, I, S, N, V, G, R, M, or Q;
AA₉, at each occurrence, is G, or S, A, L, V, W, Y, N;

R² and R³, at each occurrence, are each independently hydrogen, —CH₃.

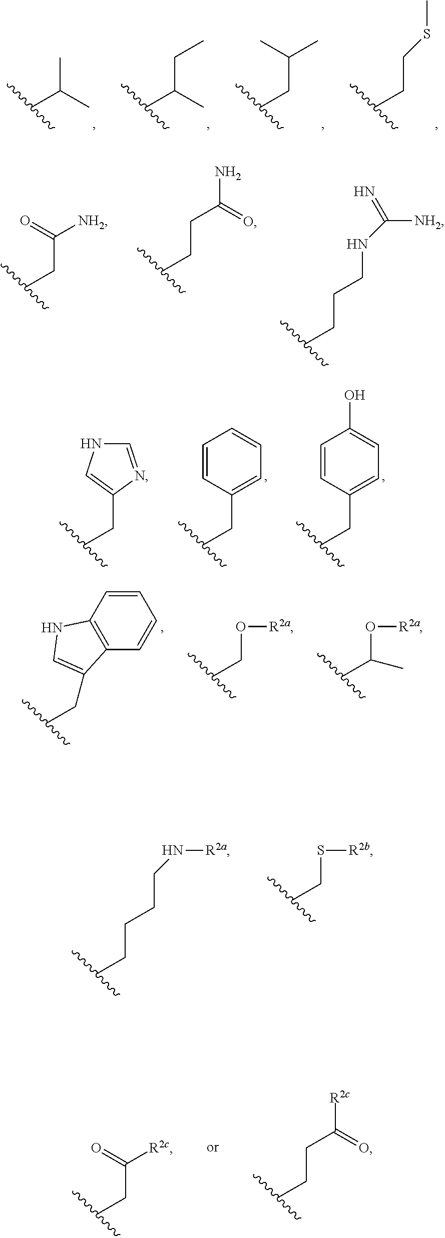

wherein:
R^{2a}, at each occurrence, is hydrogen or

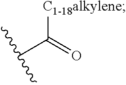

R^{2b}, at each occurrence, is hydrogen,

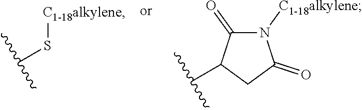

and
R^{2c}, at each occurrence, is hydrogen,

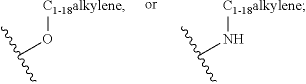

R⁴ and R⁶, at each occurrence, are each independently

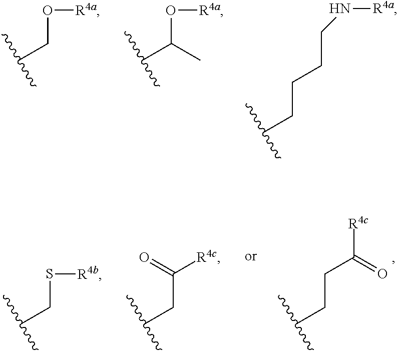

wherein:
R^{4a} is

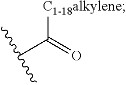

R^{4b} is

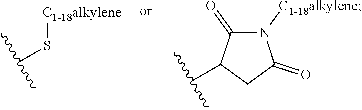

and
R^{4c} is

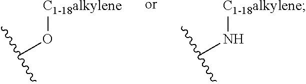

R⁵ is —OH, —O—C₁₋₁₈alkylene, or —NH—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the C₁₋₁₈alkylene is replaced with —Cy-;

R⁷ is hydrogen, or —C(O)—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of the C₁₋₁₈alkylene is replaced with —Cy-;

Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
Clause 16. The drug conjugate compound of any one of clauses 1-14, wherein the cleavable peptide moiety is a moiety of formula (II-C-A):

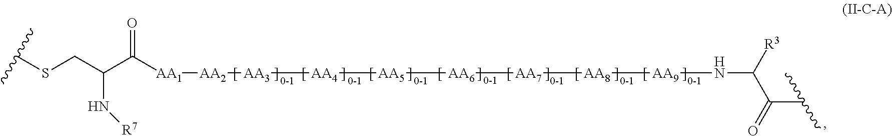

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;

R³ is hydrogen, —CH₃,

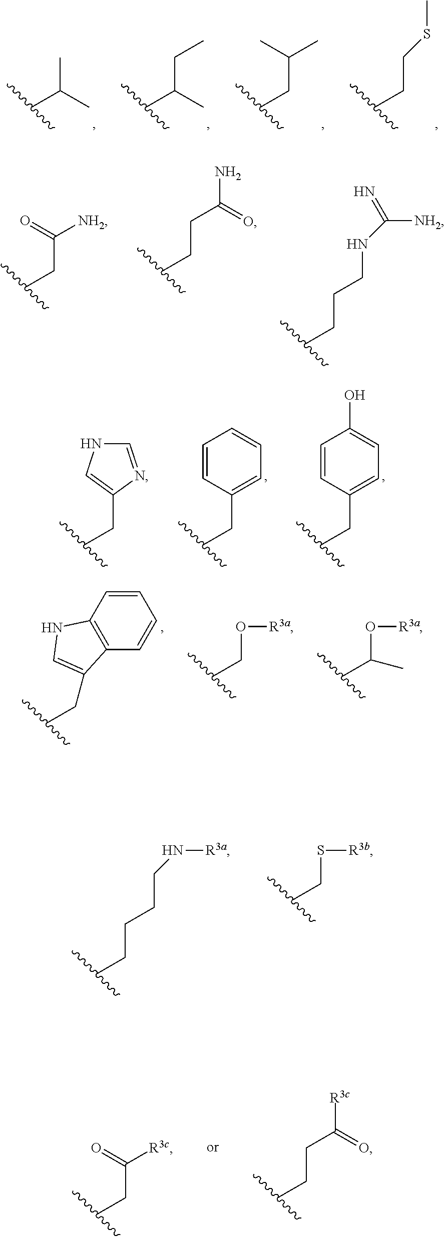

wherein:
R^{3a}, at each occurrence, is hydrogen or

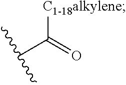

R^{3b}, at each occurrence, is hydrogen,

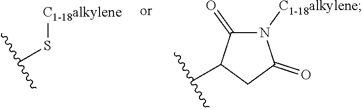

and
R^{3c}, at each occurrence, is hydrogen,

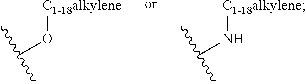

R⁷ is hydrogen, or —C(O)—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the C₁₋₁₈alkylene is replaced with —Cy-;

Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
Clause 17. The drug conjugate compound of any one of clauses 1-16, wherein the cleavable peptide moiety is a moiety of formula (III):

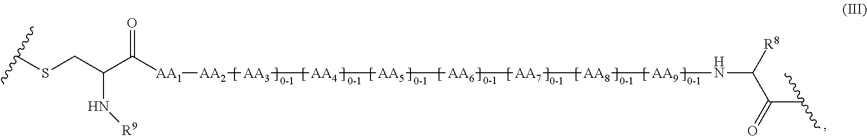

wherein:

AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;

R⁸ is hydrogen, —CH₃,

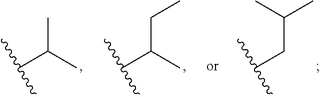

and

R⁹ is hydrogen or —C(O)CH₃.
Clause 18. The drug conjugate compound of clause 1-17, wherein the cleavable peptide moiety is a moiety of formula (III-A) or (III-B):

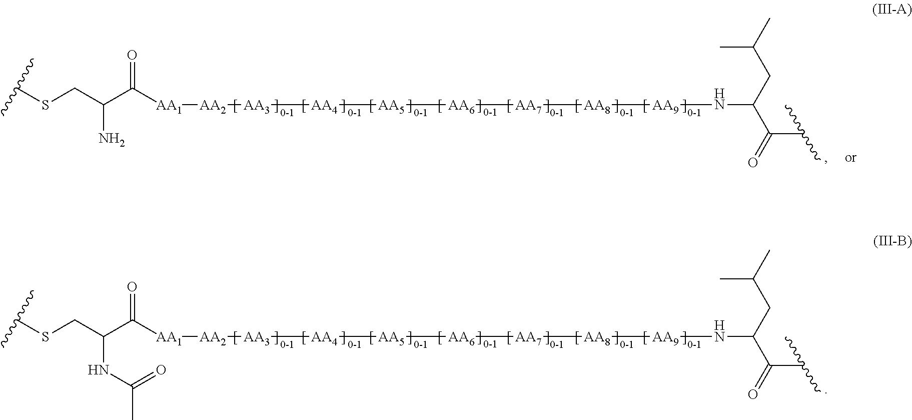

Clause 19. The drug conjugate compound of any one of clauses 1-18, wherein the cleavable peptide moiety comprises an amino acid sequence of any one of SEQ ID NO: 1-80.
Clause 20. The drug conjugate compound of any one of clauses 1-19, wherein the cleavable peptide moiety comprises at least one sequence of PLGL (SED ID NO: 4), PIGI (SED ID NO: 5), PYSI (SED ID NO: 6), PYGI (SED ID NO: 7), PYGL (SED ID NO: 8), VLSL (SED ID NO: 9), VYGL (SED ID NO: 10), VLGL (SEQ ID NO: 11); VYSL (SED ID NO: 12), PISIY (SED ID NO: 13), PSGL (SED ID NO: 14), PLGI (SED ID NO: 15), or PMAL (SED ID NO: 16).
Clause 21. The drug conjugate compound of any one of clauses 1-20, wherein the first terminus of the cleavable peptide moiety is the carboxy (C) terminus of the cleavable peptide moiety, and the second terminus of the cleavable peptide moiety is the amino (N) terminus of the cleavable peptide moiety.
Clause 22. The drug conjugate compound of any one of clauses 1-21, wherein the cleavable peptide moiety is cleavable by one or more bone marrow-enriched proteases.
Clause 23. The drug conjugate compound of clause 22, wherein at least one bone marrow-enriched protease is a matrix metalloprotease, a disintegrin and metalloproteinase, a cathepsin B protease, a cathepsin K protease, a urokinase plasminogen activator protease, or a tissue-plasminogen activator protease.
Clause 24. The drug conjugate compound of any one of clauses 1-23, wherein the bone-targeting ligand moiety is a bisphosphonate moiety of formula (IV):

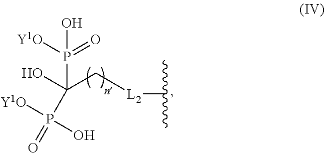

wherein:

Y¹, at each occurrence, is independently hydrogen, Na⁺, K⁺, Ca⁺², Mg⁺², or NH₄⁺;

n′ is 1-16;

L² is a C₁₋₂₄alkylene wherein optionally 1-8 methylene groups in the alkylene of L² are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of L² is replaced with —Cy-;

Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
Clause 25. The drug conjugate compound of clause 24, wherein Y¹, at each occurrence, is independently hydrogen, Na⁺, K⁺, Ca⁺², Mg⁺², or NH₄⁺.
Clause 26. The drug conjugate compound of clause 24 or 25, wherein n′ is 0-4.
Clause 27. The drug conjugate compound of any one of clauses 24-26, wherein L² comprises —N(H)—, —N(H)—C₁₋₄alkylene-, —N(CH₃)—C₁₋₄alkylene-, phenylene, imidazolylene, or triazolylene.
Clause 28. The drug conjugate compound of any one of clauses 24-27, wherein the bone-targeting ligand moiety is

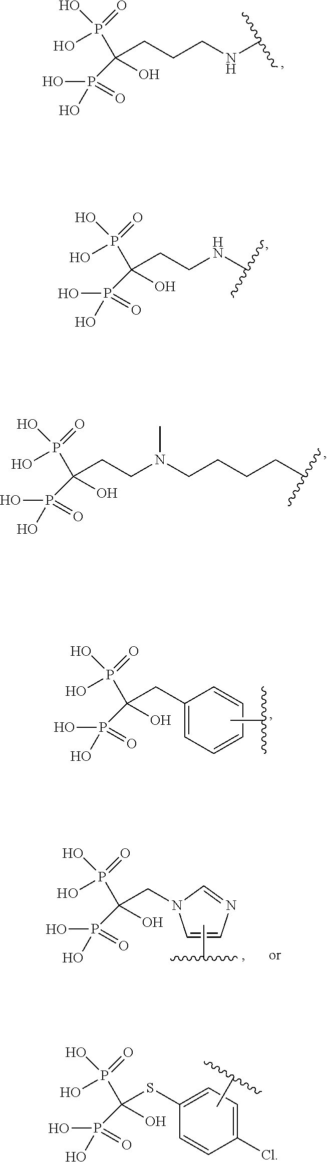

Clause 29. The drug conjugate compound of any one of clauses 1-28, wherein the second linker connects to bisphosphonate moiety by a linkage selected from the group consisting of

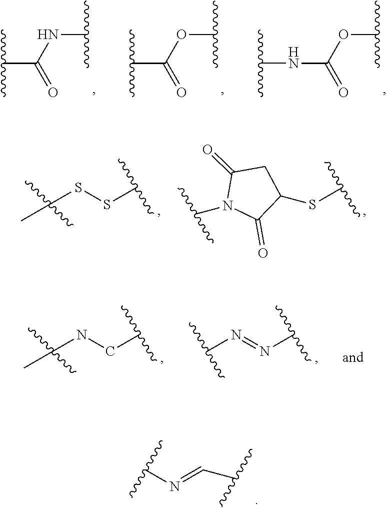

Clause 30. The drug conjugate compound of any one of clauses 1-29, wherein the second linker is a —C₁₋₂₄alkylene-wherein optionally 1-8 methylene groups in the second linker are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the second linker is replaced with-Cy-;

Cy, at each occurrence, is C₃₋₆cycloalkylene, phenylene, a 4-to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
Clause 31. The drug conjugate compound of any one of clauses 1-30, wherein the second linker comprises a maleimide moiety, wherein the maleimide moiety is attached to a cysteine moiety that is at least two amino acids away from the first terminus of the cleavable peptide moiety.
Clause 32. The drug conjugate compound of any one of clauses 1-31, wherein the second linker is a linker having formula:

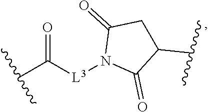

wherein:

L³ is a C₁₋₁₈alkylene wherein optionally 1-6 methylene groups in the alkylene of L³ are independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of L³ is replaced with —Cy-;

Cy, at each occurrence, is a C₃₋₆cycloalkylene, a phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and

R^{X}, at each occurrence, is independently hydrogen or C₁₋₄alkyl.
Clause 33. The drug conjugate compound of any one of clauses 1-32, wherein L³ comprises repeating units of

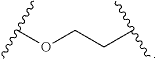

Clause 34. The drug conjugate compound of any one of clauses 1-33, wherein the second linker is:

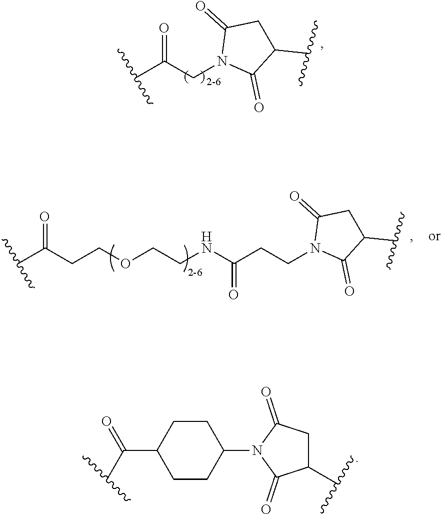

Clause 35. A pharmaceutical composition comprising the drug conjugate compound of any one of clauses 1-34 and a pharmaceutically acceptable carrier.
Clause 36. A method of targeted delivery of a protective drug to bone marrow in a subject, the method comprising administering to the subject a therapeutically effective amount of the drug conjugate compound of any one of clauses 1-34 or the pharmaceutical composition of clause 35.
Clause 37. A method of targeted delivery of a protective drug to bone marrow in a subject, the method comprising:

administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety.
Clause 38. A method of protecting a subject from radiation or chemotherapeutic toxicity, the method comprising:

administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety.
Clause 39. A method of treating or preventing a bone marrow disease in a subject, the method comprising:

administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety.
Clause 40. The method of clause 39, wherein the bone marrow disease comprises ionizing radiation-induced acute hematopoietic subsyndrome, chemotherapy-caused myelosuppression, radiotherapy-induced myelotoxicity, or combinations thereof.
Clause 41. A method of synthesizing a drug conjugate compound, the method comprising:

adding one or more protective moieties to a peptide using 2,2′-dipyridyldisulfide and N-(9-fluorenylmethoxycarbonyloxy) succinimide to generate a protected peptide;

activating the carboxy terminus of the protected peptide using N-ethyl-N-(3-(dimethylamino) propyl) carbodiimide and N-hydroxysuccinimide (NHS) to generate a peptide-NHS ester;

adding a bone marrow protective drug to the peptide-NHS ester using acetonitrile to generate a drug-conjugated peptide;

removing the one or more protective moieties from the drug-conjugated peptide using dithiothreitol and piperidine;

functionalizing a bone-targeting ligand using N-succinimidyl 6-maleimidohexanoate; and

adding the functionalized bone-targeting ligand to the drug-conjugated peptide to generate the drug conjugate compound.
Clause 42. A kit for targeted delivery of a protective drug to bone marrow in a subject, the kit comprising:

a pharmaceutical composition comprising a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety;

optionally, one or more pharmaceutically acceptable buffers, carriers, or excipients; and optionally, one or more of packaging or instructions for use.
Clause 43. Use of a drug conjugate compound for targeted delivery of a protective drug to bone marrow in a subject, the drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.
Clause 44. Use of a drug conjugate compound for protecting a subject from radiation or chemotherapeutic toxicity, the drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.
Clause 45. Use of a drug conjugate compound for treating or preventing a bone marrow disease in a subject, the drug conjugate compound comprising:

one or more bone marrow protective drug moieties;

a cleavable peptide moiety having a first terminus and a second terminus;

a bone-targeting ligand moiety;

a first linker conjugating the bone marrow protective drug moieties to the first terminus of the cleavable peptide moiety; and

a second linker conjugating the bone-targeting ligand moiety to an amino acid, where the amino acid conjugated to the bone-targeting ligand moiety is at least two amino acids away from the first terminus of the cleavable peptide moiety.

### EXAMPLES

### Example 1

### Synthesis of Bone Marrow-Specific Protective Drug Conjugates

Shown below is a general schematic route for the synthesis of the bone marrow-specific protective drug conjugates as described herein is shown below using amifostine as the protective drug and alendronate as the bone-specific targeting ligand.

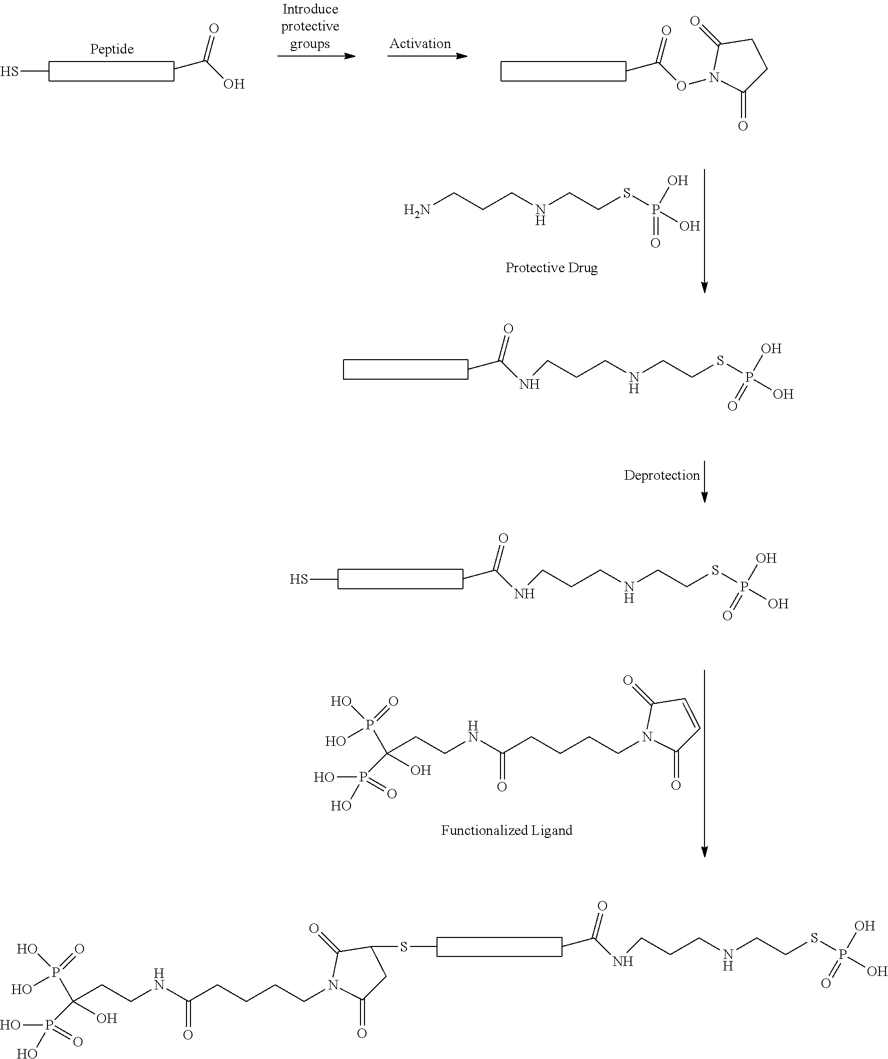

### Synthesis of a Protected Peptide

For this exemplary synthesis, 200 mg peptide having the sequence of CPIKGAIWL (SEQ D NO: 63) with acetylation (Ac) at the N-terminus (e.g., AcCPIKGAIWL; SEQ ID NO: 71) was dissolved in 5 mL of anhydrous DMF (N,N-dimethylformamide), followed by reacting with 10 mg 2,2′-dipyridyldisulfide and 50 mg Fmoc-NHS (N-(9-Fluorenylmethoxycarbonyloxy) succinimide) to protect the free thiol group of C and the primary amine group of K, respectively. The reaction mixture was mixed with a 10-fold volume of ethyl ether to precipitate out the product, followed by several washes using 20 mL of ethyl ether. After drying, the final product was stored in a white powder form (212 mg, 82%; M+1, 1374).

### Activation of the Carboxyl Group of the Protected Peptide

A total of 210 mg of the resultant protected peptide was dissolved in 5 mL anhydrous DMF and mixed with 80 mg EDC (N-ethyl-N′-(3-(dimethylamino) propyl) carbodiimide) and 30 mg NHS (N-hydroxysuccinimide) to form the peptide-NHS ester. The reaction was monitored by HPLC and was terminated when achieving the full conversion. The crude product was precipitated in 200 mL 0.1 M HCl, washed, and filtrated. The collected wet product was dried to obtain the peptide-NHS ester in a fine powder form (173 mg, 76%; M+1, 1471).

### Synthesis of the Peptide-Amifostine Conjugate and the Deprotection

A total of 170 mg of the peptide-NHS ester was dissolved in 30 mL acetonitrile and then added into an aqueous solution of amifostine (100 mg, 30 mL). The reaction was monitored by HPLC until all the peptide-NHS was converted. Subsequently, dithiothreitol (50 mg) and piperidine (5 mL) were added to the mixture to allow the removal of the two protective groups. The final product peptide-amifostine conjugate was isolated by flash chromatography (112 mg, 84%; (M+2)/2, 620).

### Synthesis of Maleimide-Functionalized Alendronate (Maleimide-C₆-Alendronate)

A total of 225 mg of alendronate trihydrate was dissolved in 20 mL aqueous solution with the pH adjusted to 8 using 1 M NaOH. Maleimide-C6-NHS (300 mg) was dissolved in 6 mL acetonitrile, followed by mixing with the aqueous solution of alendronate. Upon completion of the reaction, the liquid of the solution was removed under reduced pressure, and the resultant powder was washed with abundant acetonitrile several times. The crude product was dried under a vacuum condition to obtain Maleimide-C₆-Alendronate (348 mg, 92%; M-1, 443).

### Synthesis of the Bone Marrow-Specific Protective Drug Conjugate

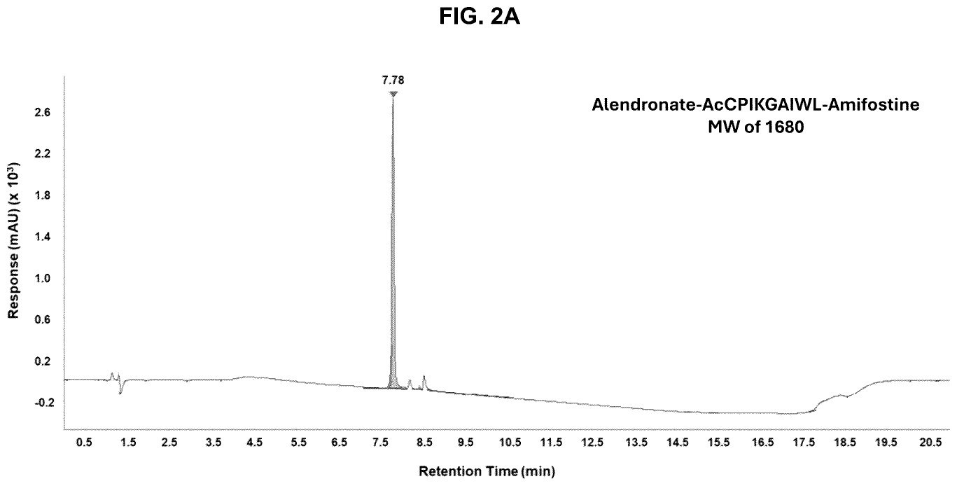

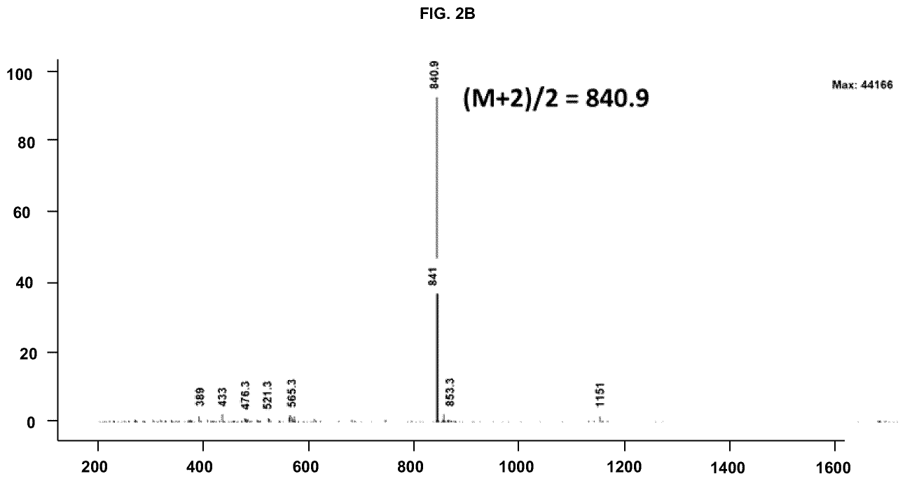

The peptide-amifostine conjugate (110 mg) and maleimide-functionalized alendronate (100 mg) were dissolved in 20 mL of acetonitrile/water (volume/volume, 1/1) mixture. After the full conversion of the peptide-amifostine conjugate, the final product was separated out using flash chromatography and lyophilized to fiber-like powder (114 mg, 76%; (M+2)/2, 841). FIG. 2A-B show the representative HPLC (the peak with a retention time of 7.78 min) and LC/MS spectra of the final product with a purity of 96.2%.

### Example 2

### Synthesis of a Protective Drug Conjugate with Peptide Sequence of CPGFLGIA

For this exemplary synthesis, 150 mg peptide having the sequence of CPGFLGIA (SEQ ID NO: 51) was dissolved in 10 mL of anhydrous methanol, followed by reacting with 15 mg 2,2′-dipyridyldisulfide and 80 mg Fmoc-NHS to protect the free thiol group and the primary amine groups, respectively. The reaction mixture was mixed with a 10-fold volume of ethyl ether to precipitate out the product, followed by several washes. After drying, the final product was dissolved in 5 mL anhydrous DMF and mixed with 70 mg EDC and 30 mg NHS to form the peptide-NHS ester. The reaction was monitored by HPLC and was terminated when achieving the full conversion. The crude product was precipitated in 200 mL 0.1 M HCl, washed, and filtrated. The collected wet product was dissolved in 30 mL acetonitrile and then added into an aqueous solution of amifostine (100 mg, 30 mL). The reaction was monitored by HPLC until all the peptide-NHS was converted. Subsequently, dithiothreitol (50 mg) and piperidine (5 mL) were added to the mixture to allow the removal of the two protective groups. The final peptide-amifostine conjugate product was isolated by flash chromatography, followed by reacting with Maleimide-C₆-Alendronate (100 mg) in 20 mL of acetonitrile/water (volume/volume, 1/1) mixture. After the full conversion of the peptide-amifostine conjugate, the final product was separated out using flash chromatography and lyophilized to fiber-like powder (120 mg, 44%; (M+2)/2, 709).

### Example 3

### Synthesis of a Protective Drug Conjugate with Peptide Sequence of AcCGQVYGLAIL

For this exemplary synthesis, 250 mg peptide having the sequence of CGQVYGLAIL (SEQ ID NO: 64) with acetylation (Ac) at the N-terminus (e.g., AcCGQVYGLAIL; SEQ ID NO: 72) was dissolved in 20 ml of anhydrous methanol, followed by reacting with 30 mg 2,2′-dipyridyldisulfide to protect the free thiol group. The reaction mixture was mixed with a 10-fold volume of ethyl ether to precipitate out the product, followed by several washes. After drying, the final product was dissolved in 5 mL anhydrous DMF and mixed with 80 mg EDC and 30 mg NHS to form the peptide-NHS ester. The reaction was monitored by HPLC and was terminated when achieving the full conversion. The crude product was precipitated in 200 mL 0.1 M HCl, washed twice, and filtrated. The collected wet product was dissolved in 30 mL acetonitrile and then added into an aqueous solution of amifostine (150 mg, 30 mL). The reaction was monitored until all the peptide-NHS was converted. Subsequently, dithiothreitol (50 mg) was added to the mixture to allow the removal of the protective group. The final peptide-amifostine conjugate product was isolated by flash chromatography, followed by reacting with Maleimide-C₆-Alendronate (100 mg) in 20 mL of acetonitrile/water (volume/volume, 1/1) mixture. After the full conversion of the peptide-amifostine conjugate, the final product was separated out using flash chromatography and lyophilized to fiber-like powder (219 mg, 55%; M+1, 1718).

### Example 4

### Synthesis of a Protective Drug Conjugate with Peptide Sequence of AcCFRL

For this exemplary synthesis, 220 mg peptide having the sequence of CFRL (SEQ ID NO: 45) with acetylation (Ac) at the N-terminus (e.g., AcCFRL; SEQ ID NO: 73) was dissolved in 20 mL of anhydrous methanol, followed by reacting with 25 mg 2,2′-dipyridyldisulfide to protect the free thiol group. The reaction mixture was mixed with a 10-fold volume of ethyl ether to precipitate out the product, followed by several washes. After drying, the final product was dissolved in 5 mL anhydrous DMF and mixed with 80 mg EDC and 30 mg NHS to form the peptide-NHS ester. The reaction was monitored by HPLC and was terminated when achieving the full conversion. The crude product was precipitated using a 10-fold volume of ethyl ether and then dissolved in dichloromethane, followed by washing with acidified saturated NaCl solution. Subsequently, the organic phase was dried with Na₂SO₄ and evaporated dichloromethane under reduced pressure. The resultant product was dissolved in 30 mL acetonitrile and then added into an aqueous solution of amifostine (120 mg, 30 mL). The reaction was monitored until all the peptide-NHS was converted. Subsequently, dithiothreitol (50 mg) was added to the mixture to allow the removal of the protective group. The final peptide-amifostine conjugate product was isolated by flash chromatography, followed by reacting with Maleimide-C₆-Alendronate (100 mg) in 20 mL of acetonitrile/water (volume/volume, 1/1) mixture. After the full conversion of the peptide-amifostine conjugate, the final product was separated out using flash chromatography and lyophilized to fiber-like powder (291 mg, 63%; (M+2)/2, 610).

### Example 5

### Synthesis of Maleimide-Functionalized Alendronate (Maleimide-PEG₂-Alendronate)

For this exemplary synthesis, a total of 225 mg of alendronate trihydrate was dissolved in 20 mL aqueous solution with the pH adjusted to 8 using 1 M NaOH. Maleimide-PEG₂-NHS (380 mg) was dissolved in 6 mL acetonitrile, followed by mixing with the aqueous solution of alendronate. Upon completion of the reaction, the liquid of the solution was removed under reduced pressure, and the resultant powder was washed with abundant acetonitrile several times.

The crude product was dried under a vacuum condition to obtain Maleimide-PEG₂-Alendronate (440 mg, 87%; M-1, 559).

### Example 6

Synthesis of a Protective Drug Conjugate with Peptide Sequence of CALRGAI

For this exemplary synthesis, 200 mg peptide having the sequence of CALRGAI (SEQ ID NO: 48) was dissolved in 10 mL of anhydrous methanol, followed by reacting with 10 mg 2,2′-dipyridyldisulfide and 80 mg Fmoc-NHS to protect the free thiol group and the primary amine group, respectively. The reaction mixture was mixed with a 10-fold volume of ethyl ether to precipitate out the product, followed by several washes. After drying, the final product was dissolved in 5 mL anhydrous DMF and mixed with 90 mg EDC and 40 mg NHS to form the peptide-NHS ester. The reaction was monitored by HPLC and was terminated when achieving the full conversion. The crude product was precipitated in 200 mL 0.1 M HCl, washed, and filtrated. The collected wet product was dissolved in 30 mL acetonitrile and then added into an aqueous solution of amifostine (100 mg, 30 mL). The reaction was monitored by HPLC until all the peptide-NHS was converted. Subsequently, dithiothreitol (50 mg) and piperidine (5 mL) were added to the mixture to allow the removal of the two protective groups. The final peptide-amifostine conjugate product was isolated by flash chromatography, followed by reacting with Maleimide-PEG2-Alendronate (180 mg) in 20 mL of acetonitrile/water (volume/volume, 1/1) mixture. After the full conversion of the peptide-amifostine conjugate, the final product was separated out using flash chromatography and lyophilized to fiber-like powder (199 mg, 48%; (M+3)/3, 487).

### Example 7

### Synthesis of a Protective Drug Conjugate with Peptide Sequence of AcCPFGFLGIAL

For this exemplary synthesis, 250 mg peptide having the sequence of CPFGFLGIAL (SEQ ID NO: 52) with acetylation (Ac) at the N-terminus (e.g., AcCPFGFLGIAL; SEQ ID NO: 77) was dissolved in 20 mL of anhydrous methanol, followed by the reaction with 25 mg 2,2′-dipyridyldisulfide to protect the free thiol group. The reaction mixture was mixed with a 10-fold volume of ethyl ether to precipitate out the product, followed by several washes. After drying, the final product was dissolved in 5 mL anhydrous DMF and mixed with 80 mg EDC and 30 mg NHS to form the peptide-NHS ester. The reaction was monitored by HPLC and was terminated when achieving the full conversion. The crude product was precipitated using a 10-fold volume of ethyl ether and then dissolved in dichloromethane, followed by washing with acidified saturated NaCl solution. Subsequently, the organic phase was dried with Na₂SO₄ and evaporated dichloromethane under reduced pressure. The resultant product was dissolved in 30 mL acetonitrile and then added into an aqueous solution of amifostine (120 mg, 30 mL). The reaction was monitored until all the peptide-NHS was converted. Subsequently, dithiothreitol (50 mg) was added to the mixture to allow the removal of the protective group. The final peptide-amifostine conjugate product was isolated by flash chromatography, followed by reacting with Maleimide-C₆-Alendronate (100 mg) in 20 mL of acetonitrile/water (volume/volume, 1/1) mixture. After the full conversion of the peptide-amifostine conjugate, the final product was separated out using flash chromatography and lyophilized to fiber-like powder (161 mg, 38%; M+1, 1836).

### Example 8

### In Vivo Radioprotective Effects of the Bone Marrow-Specific Conjugates

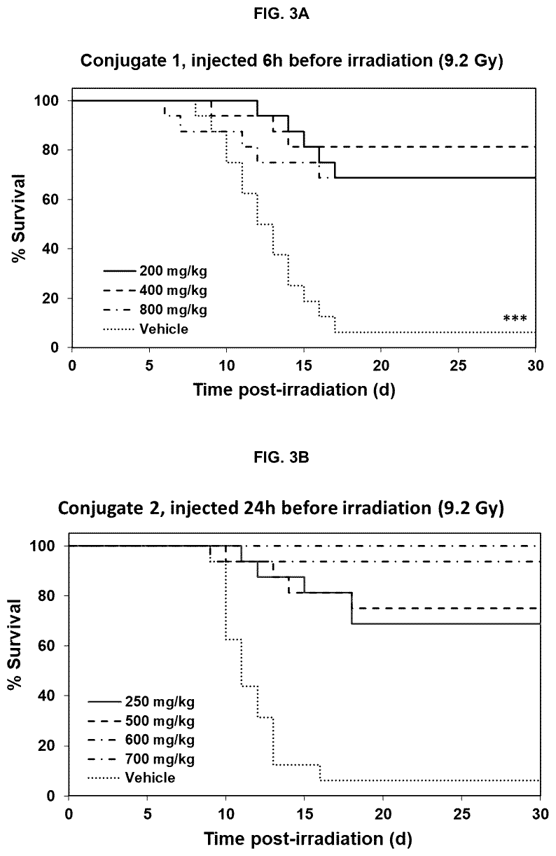

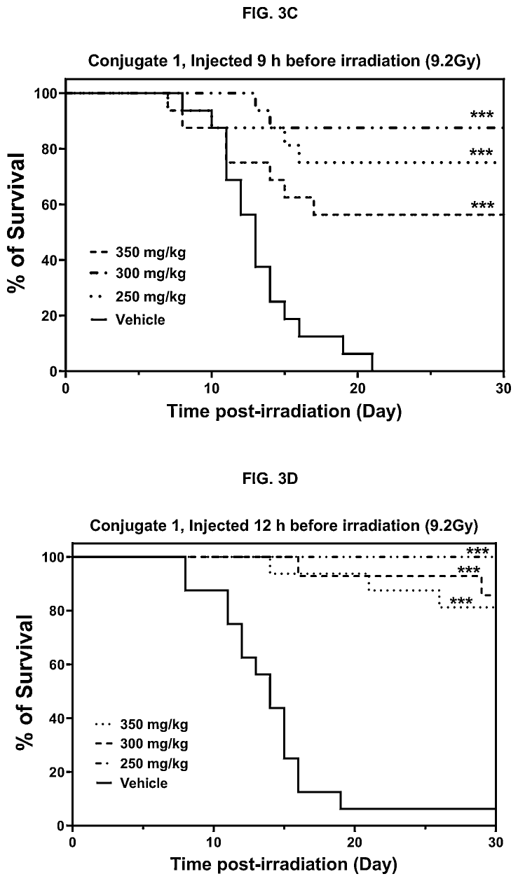

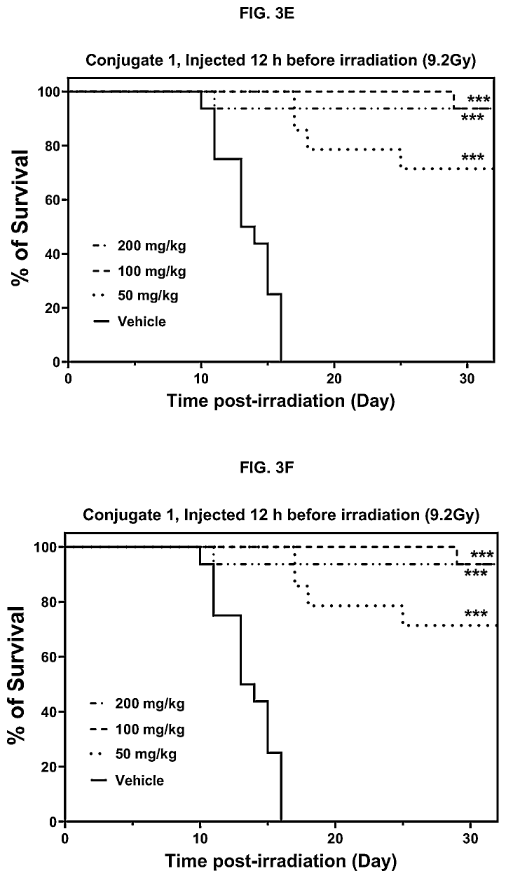

CD2F1 male mice (N=16) were subcutaneously (sc) injected with a single dose of two different conjugates. For one protective drug conjugate (Conjugate 1), the tested doses were 200, 400, and 800 mg/kg. The structure of Conjugate 1 included amifostine as the protective drug, alendronate as the bone-targeting ligand, and a protease-cleavable peptide having the sequence of CKPLGLWAL (SEQ ID NO: 33) with acetylation at the N-terminus (e.g., AcCKPLGLWAL; SEQ ID NO: 74). After 6 h from drug administration, mice were exposed to a dose of 9.2 Gy Cobalt-60 gamma-ray, which was demonstrated to induce the hematopoietic acute radiation subsyndrome. The irradiated mice were monitored for survival for 30 days post-irradiation. FIG. 3A shows the 30-day survival curves of mice in each group. For the vehicle control group, the mortalities of mice occurred during 8 to 17 days post-irradiation, and the final survival rate was 6.25% (1 out of 16 survived), indicating significant mortality by gamma-irradiation. Comparatively, all the groups of mice administered with the protective drug conjugate showed significant survival benefits, i.e., the survival rates for the administered doses of 200, 400,and 800 mg/kg were improved to 68.75% (11 out of 16), 81.25% (13 out of 16), and 68.75% (11 out of 16), respectively.

For another protective drug conjugate (Conjugate 2), the tested doses were 250, 500, 600, and 700 mg/kg, and the irradiation was conducted at 24 h post-drug administration. The structure of Conjugate 2 included amifostine as the protective drug, alendronate as the bone-targeting ligand, and a protease-cleavable peptide having the sequence of CPLGLYAL (SEQ ID NO: 29) with acetylation at the N-terminus (e.g., AcCPLGLYAL; SEQ ID NO: 76). FIG. 3B shows the 30-day survival curves of mice in each group. Compared to the vehicle control (30-day survival rate of 6.25%), all the groups of mice administered with Conjugate 2 showed significant survival benefits, i.e., the survival rates for the drug doses of 250, 500, 600, and 700 mg/kg were improved to 68.75% (11 out of 16), 75% (12 out of 16), 93.75% (15 out of 16), and 100% (16 out of 16), respectively.

### Example 9

### In Vivo Radioprotective Effects of the Bone Marrow-Specific Conjugates

CD2F1 male mice (N=16) were subcutaneously (sc) injected with a single dose of a protective drug conjugate (Conjugate 1), and the tested doses were 250, 300, and 350 mg/kg. The structure of Conjugate 1 included amifostine as the protective drug, alendronate as the bone-targeting ligand, and a protease-cleavable peptide having the sequence of CKPLGLWAL (SEQ ID NO: 33) with acetylation at the N-terminus (e.g., AcCKPLGLWAL; SEQ ID NO: 74). After 9 h from drug administration, mice were exposed to a dose of 9.2 Gy Cobalt-60 gamma-ray, which was demonstrated to induce the hematopoietic acute radiation subsyndrome. The irradiated mice were monitored for survival for 30 days post-irradiation. FIG. 3C shows the 30-day survival curves of mice in each group. For the vehicle control group, the mortalities of mice occurred during 8 to 17 days post-irradiation, and the final survival rate was 0% (0 out of 16 survived), indicating significant mortality by gamma-irradiation. Comparatively, all the groups of mice administered with the protective drug conjugate showed significant survival benefits, i.e., the survival rates for the administered doses of 250, 300, and 350 mg/kg were improved to 75% (12 out of 16), 87.25% (14 out of 16), and 56.25% (9 out of 16), respectively.

### Example 10

### In Vivo Radioprotective Effects of the Bone Marrow-Specific Conjugates

CD2F1 male mice (N=16) were subcutaneously (sc) injected CD2F1 male mice (N=16) were subcutaneously (sc) injected with a single dose of a protective drug conjugate (Conjugate 1), and the tested doses were 250, 300, and 350 mg/kg. The structure of Conjugate 1 included amifostine as the protective drug, alendronate as the bone-targeting ligand, and a protease-cleavable peptide having the sequence of CKPLGLWAL (SEQ ID NO: 33) with acetylation at the N-terminus (e.g., AcCKPLGLWAL; SEQ ID NO: 74). After 12 h from drug administration, mice were exposed to a dose of 9.2 Gy Cobalt-60 gamma-ray, which was demonstrated to induce the hematopoietic acute radiation subsyndrome. The irradiated mice were monitored for survival for 30 days post-irradiation. FIG. 3D shows the 30-day survival curves of mice in each group. For the vehicle control group, the mortalities of mice occurred during 8 to 17 days post-irradiation, and the final survival rate was 6.25% (1 out of 16 survived), indicating significant mortality by gamma-irradiation. Comparatively, all the groups of mice administered with the protective drug conjugate showed significant survival benefits, i.e., the survival rates for the administered doses of 250, 300, and 350 mg/kg were improved to 100% (16 out of 16), 87.5% (14 out of 16), and 81.25% (13 out of 16), respectively.

### Example 11

### In Vivo Radioprotective Effects of the Bone Marrow-Specific Conjugates

CD2F1 male mice (N=16) were subcutaneously (sc) injected CD2F1 male mice (N=16) were subcutaneously (sc) injected with a single dose of a protective drug conjugate (Conjugate 1), and the tested doses were 50, 100, and 200 mg/kg. The structure of Conjugate 1 included amifostine as the protective drug, alendronate as the bone-targeting ligand, and a protease-cleavable peptide having the sequence of CKPLGLWAL (SEQ ID NO: 33) with acetylation at the N-terminus (e.g., AcCKPLGLWAL; SEQ ID NO: 74). After 12 h from drug administration, mice were exposed to a dose of 9.1 Gy Cobalt-60 gamma-ray, which was demonstrated to induce the hematopoietic acute radiation subsyndrome. The irradiated mice were monitored for survival for 32 days post-irradiation. FIG. 3E shows the 30-day survival curves of mice in each group. For the vehicle control group, the mortalities of mice occurred during 8 to 17 days post-irradiation, and the final survival rate was 0% (0 out of 16 survived), indicating significant mortality by gamma-irradiation. Comparatively, all the groups of mice administered with the protective drug conjugate showed significant survival benefits, i.e., the survival rates for the administered doses of 50, 100, and 200 mg/kg were improved to 75% (16 out of 16), 93.75% (15 out of 16), and 93.75% (15 out of 16), respectively.

### Example 12

### In Vivo Radioprotective Effects of the Bone Marrow-Specific Conjugates

CD2F1 male mice (N=16) were subcutaneously (sc) injected CD2F1 male mice (N=16) were subcutaneously (sc) injected with a single dose of a protective drug conjugate, i.e., Conjugate 3. The tested doses were 250, 300, and 350 mg/kg. The structure of Conjugate 3 included amifostine as the protective drug, alendronate as the bone-targeting ligand, and a protease-cleavable peptide having the sequence of CKPLGLWAL (SEQ ID NO: 33) with acetylation at the N-terminus (e.g., AcCKPLGLWAL; SEQ ID NO: 74). Note that the thiol-maleimide ring in Conjugate 3 was 100% hydrolyzed. After 6 h from drug administration, mice were exposed to a dose of 9.2 Gy Cobalt-60 gamma-ray, which was demonstrated to induce the hematopoietic acute radiation subsyndrome. The irradiated mice were monitored for survival for 30 days post-irradiation. FIG. 3F shows the 30-day survival curves of mice in each group. For the vehicle control group, the mortalities of mice occurred during 8 to 17 days post-irradiation, and the final survival rate was 0% (0 out of 16 survived), indicating significant mortality by gamma-irradiation. Comparatively, all the groups of mice administered with the protective drug conjugate showed no survival benefits. The hydrolyzed thiol-maleimide moiety in conjugates prevented the retro-Michael addition, which plays a role in the faster release of protective drugs.

### Example 13

### Sequence-Dependent Conjugate Cleavage in Fresh Bone Marrow Aspirate

The proteolytic cleavage of conjugates determines the therapeutic window, which is controlled by the specific peptide sequence. In a representative cleavage experiment, a 10 L stock solution of a conjugate (30 mg/mL) was mixed with 90 μL of fresh bone marrow aspirate, followed by incubation at 37° C. for up to 6 h. A 10 μL sample was taken every hour, diluted in 90 μL acetonitrile to precipitate proteins and extract conjugates and cleavage products. The concentrations of the conjugates in samples were determined by HPLC, normalized, and presented as the cleavage ratio.

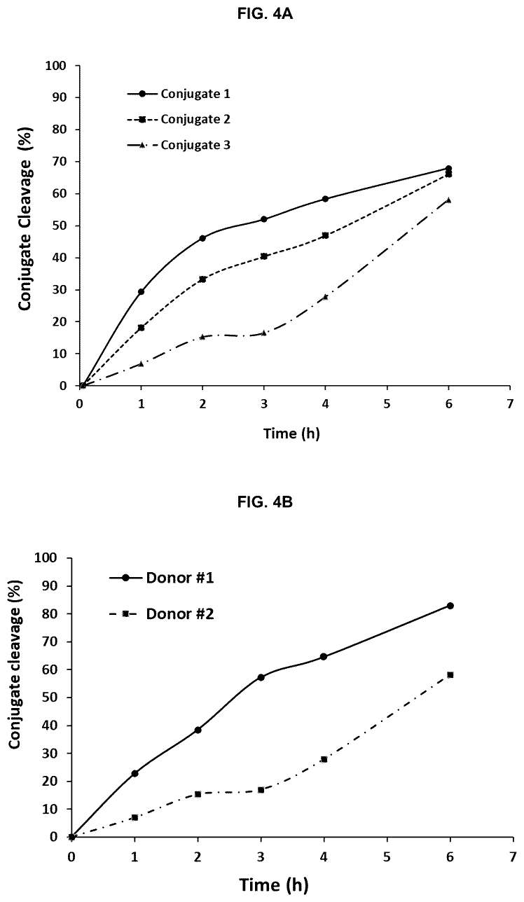

FIG. 4A shows the cleavage of three conjugates having different peptide sequences (Conjugates 1-3). The structures of each conjugate included amifostine as the protective drug, alendronate as the bone-targeting ligand, and a protease-cleavable peptide having the following amino acid sequences: Conjugate 1-CKPLGLWAL (SEQ ID NO: 33), Conjugate 2-CQPLGLWAL (SEQ ID NO: 69), and Conjugate 3-CPLGLYAL (SEQ ID NO: 29). Each peptide of Conjugates 1-3 was acetylated (Ac) at the N-terminus (e.g., AcCKPLGLWAL, SEQ ID NO: 74; AcCQPLGLWAL, SEQ ID NO: 75; and AcCPLGLYAL, SEQ ID NO: 76, respectively). The time to reach 50% conjugate cleavage was 2.4 h, 4.2 h, and 5.5 h for Conjugate 1, Conjugate 2, and Conjugate 3, respectively. This demonstrated that the proteolytic cleavage of these exemplary bone marrow-specific conjugates was peptide sequence-dependent.

Moreover, the cleavage rate differed between different donors. As shown in FIG. 4B, the cleavage rate of Conjugate 3 was much higher when incubated in the bone marrow aspirate from Donor 1 as compared to Donor 2, which may have been attributed to differences in bone marrow cell density between the two donors. For example, the cell density in the bone marrow aspirate from Donor 1 was relatively higher than the cell density in the bone marrow aspirate from Donor 2 (2.5×10⁶/mL versus 1.8×10⁶/mL).

### Example 14

### Conjugate Binding to Hydroxyapatite

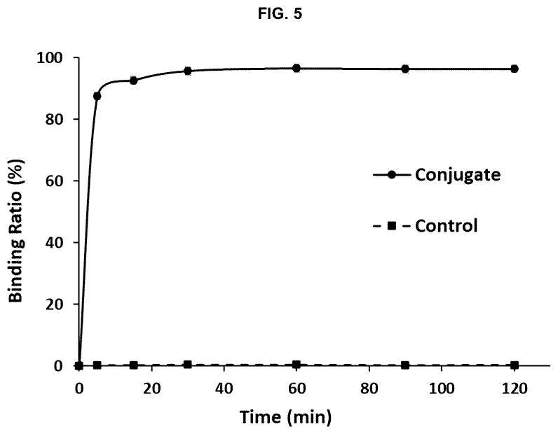

The binding of the protective drug conjugate with hydroxyapatite was evaluated to verify the bone surface-specific accumulation. In brief, a total of 100 μL conjugate solution (1 mg/mL) having a peptide sequence of CPISIAL (SEQ ID NO: 70) with acetylation at the N-terminus (e.g., AcCPISIAL; SEQ ID NO: 79) was added into 0.9 mL suspension containing 10 mg hydroxyapatite powder, followed by vortexing to form a homogeneous mixture. At a pre-determined time point (i.e., 5, 15, 30, 60, 90, or 120 min), a 100 μL sample was taken and centrifuged to collect the supernatant. The quantity of the conjugate left in the supernatant was determined, normalized, and presented as the binding ratio. As shown in FIG. 5, the binding of the conjugate to hydroxyapatite reached equilibrium (almost 100%) within less than 20 min. Comparatively, the control group, i.e., the conjugate without an alendronate ligand, showed no binding with hydroxyapatite, even after 120 min of incubation.

### Example 15

### Conjugate Concentration-Dependent In Vitro Radioprotection for Human HSPCs

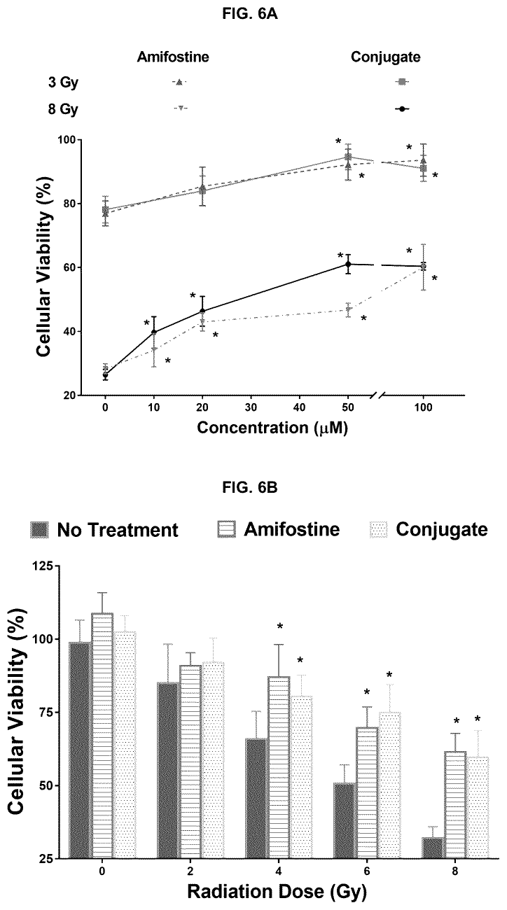

In this exemplary representative experiment, human HSPCs were seeded in a 96-well plate at a density of 2.5×10⁴ per well (N=6 for each group). Next, cells were treated with a protective drug conjugate having a peptide sequence of CAPIGIAL (SEQ ID NO: 68) with acetylation at the N-terminus (e.g., AcCAPIGIAL; SEQ ID NO: 80) or amifostine alone at the final concentrations ranging from 0 to 100 μM for 4 h, followed by exposure to y-radiation (Cs-137) at 3 or 8 Gy (0.6 Gy/min). Two hours after the irradiation, the culture medium was replaced by a fresh medium. After 24 h of further incubation, a CCK8 assay was applied to evaluate the cell viability. As shown in FIG. 6A, for the control group without treatment of any protective agent (i.e., 0 μM conjugate or amifostine), 8 Gy irradiation dramatically reduced the cellular viability to ˜26.3%. Compared to this, cells pre-treated with the Peptide-Amifostine conjugate demonstrated significantly higher viability. The maximum radioprotective efficacy was observed at the concentration of 50 μM, which led to the cellular viability of 61.1%, i.e., ˜2.3-fold greater than the control group. Compared to the irradiation dose of 8 Gy, the lower radiation dose, i.e., 3 Gy, moderately reduced the viability to 78.1% in the control group, and a significant radioprotective effect was observed when cells were pre-treated with the protective Peptide-Amifostine conjugate at concentrations of 50 or 100 μM. A t-test was applied for data analyses, and statistically significant radioprotection (P<0.05) was observed at all concentrations with 8 Gy exposure, and for 50 and 100 μM drug concentrations with 3 Gy exposure, when compared to the data with no treatment (i.e., 0 M conjugate or amifostine).

### Example 16

Irradiation Dose-Dependent In Vitro Radioprotection for Human HSPCs

In this exemplary representative experiment, human HSPCs were seeded in a 96-well plate at a density of 2.5×10⁴ per well (N=6 for each group). Next, cells were treated with a protective drug conjugate having a peptide sequence of CAPIGIAL (SEQ ID NO: 68) with acetylation at the N-terminus (e.g., AcCAPIGIAL; (SEQ ID NO: 80) or amifostine alone with a fixed concentration of 50 μM for 4 h, followed by the exposure to γ-radiation (Cs-137) with the irradiation doses of 0, 2, 4, 6, or 8 Gy (0.6 Gy/min). Two hours after the irradiation, the culture medium was replaced by a fresh medium. After 24 h of further incubation, a CCK8 assay was applied to evaluate the cell viability. As shown in FIG. 6B, cells pre-treated with the conjugate or amifostine alone showed much higher cellular viability compared to the group receiving no treatment at the same exposure dose, while the radioprotective effect was more significant when the irradiation dose was increased (i.e., 4, 6, or 8 Gy). For instance, pre-treatment with the conjugate improved the cellular viability from 84.9% to 91.9% under the 2 Gy radiation exposure, while from 32.1% to 59.6% under the 8 Gy radiation exposure. In addition, for the conjugate and amifostine groups, no statistical difference in cellular viability (i.e., P>0.05) was determined at any exposure doses (from 2 Gy to 8 Gy), thus indicating the comparable radioprotection effect of the conjugate to amifostine alone.

### Example 17

### In Vitro Matrix Metalloproteinase-Based Conjugate Cleavage

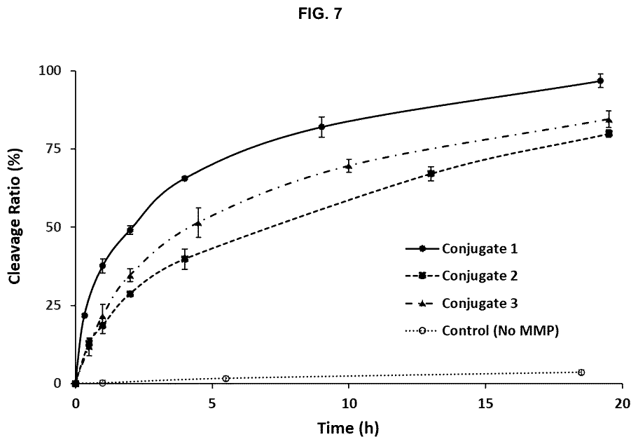

In this exemplary representative cleavage experiment, a 90 μL stock solution of protective drug conjugate (1 mg/mL) was mixed with 2 μL of matrix metalloproteinase 9 (MMP9) (10 unit/μL) and 8.2 μL of 50 mM CaCl₂ solution, followed by incubation at 37° C. for up to 20 h. A 10 μL sample was taken at 0.3, 1, 2, 4, 9, or 20 h for the HPLC analysis. Three conjugates having different peptide sequences (Conjugates 1-3) were used in this test: Conjugate 1-peptide sequence of CPLSLYAL (SEQ ID NO: 65); Conjugate 2-peptide sequence of CPLSLYSL (SEQ ID NO: 66); and Conjugate 3-peptide sequence of CPLGLAL (SEQ ID NO: 67). The concentrations of the conjugate residual in samples were normalized and presented as cleavage ratios. As shown in FIG. 7, the three different protective drug conjugates incubated with MMP9 resulted in significant proteolytic cleavages compared to the control group, i.e., Conjugate 1 incubated at the same condition without MMP. The different cleavage rates demonstrated a clear correlation with the different peptide sequences of each conjugate, where Conjugate 1 had the fastest cleavage rate (T_{1/2}˜2 h) and Conjugate 2 had the slowest cleavage rate (T_{1/2}˜7.5 h).

### Example 18

### In Vitro Protection of Bone Marrow Mononuclear Cells Exposed to Chemodrugs

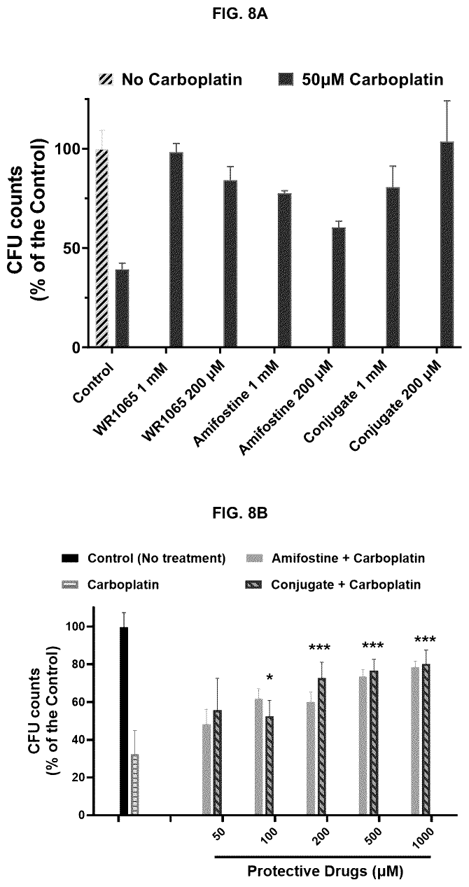

In this exemplary representative chemoprotection experiment, a colony forming unit assay was conducted for bone marrow mononuclear cells exposed to carboplatin with or without the pre-treatment with protective agents. In brief, 1.1×10⁴ human bone marrow mononuclear cells were suspended in 300 μL culture medium and mixed with 30 μL solutions of PBS (control), Amifostine, WR1065, or Conjugate (with the peptide sequence of CFLG (SEQ ID NO: 18)) at various concentrations. After 2 h of incubation, cells were washed and resuspended in 300 μL culture medium, followed by incubation with 50 μM carboplatin for 4 h. Next, cells were washed and resuspended in the colony forming medium for 14 days. The number of resultant CFU in each group was then determined, and the results were normalized to the group of cells without any treatment. As shown in FIG. 8A, cells exposed to 50 μM carboplatin for 4 h dramatically reduced the CFU counts to ˜40% (a reduction of ˜60%). However, when cells were pretreated by 1 mM or 200 μM (clinically relevant concentrations) protective drugs, including Amifostine, WR1065, or Conjugates, the subsequent exposure to 50 μM carboplatin only slightly reduced the CFU counts, e.g., the reduction was in the range of 2-20%. At the low concentration, e.g., 200 μM protective drugs, the Conjugate showed better protective effects than amifostine, which could probably be attributed to the enhanced cellular internalization of the Conjugate (a much higher lipophilicity than amifostine). These findings demonstrated that Conjugate had comparable or even better chemoprotective effects for bone marrow mononuclear cells to the parent drug amifostine. In another exemplary representative chemoprotection experiment, Amifostine and Conjugate with the peptide sequence of CFLG (SEQ ID NO: 18) with concentrations ranging from 50 to 1,000 μM were evaluated. As shown in FIG. 8B, cells exposed to 50 μM carboplatin for 4 h dramatically reduced the CFU counts to ˜30% (a reduction of ˜70%). However, the pretreatment with Amifostine or Conjugates significantly mitigated Carboplatin-caused CFU reduction in a dose-dependent manner. No statistically significant difference was found in the protective effects between Amifostine and the Conjugate, indicating that Conjugate exhibited comparable chemoprotective properties for bone marrow mononuclear cells when compared to the parent drug, Amifostine.

### Example 19

### In Vitro Proliferation and Differentiation Stimulation of Bone Marrow Mononuclear Cells

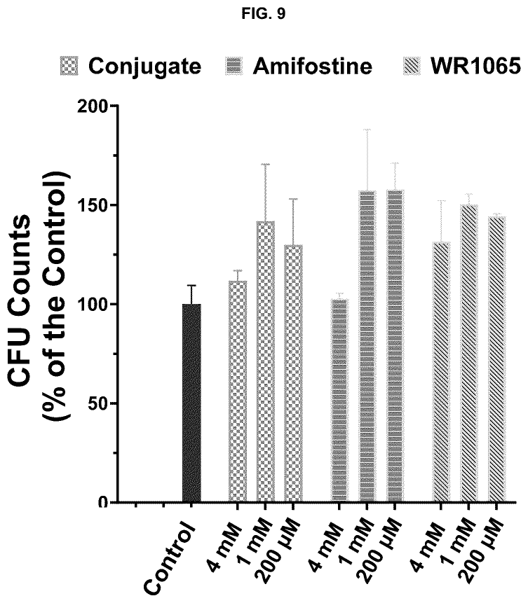

In this exemplary representative experiment, a colony-forming unit assay was conducted for bone marrow mononuclear cells exposed to protective agents. In brief, 1.1×10⁴ human bone marrow mononuclear cells were suspended in 300 μL culture medium and mixed with 30 μL solutions of PBS (control), Amifostine, WR1065, or Conjugate (with the peptide sequence of CFLG (SEQ ID NO: 18)) at various concentrations. After 2 h of incubation, cells were washed and resuspended in 300 μL culture medium, followed by incubation with 50 μM carboplatin for 4 h. Next, cells were washed and resuspended in the colony-forming medium for 14 days. The number of resultant CFU in each group was then determined, and the results were normalized to the group of cells without any treatment. As shown in FIG. 9, cells exposed to 1 mM or 200 μM (clinically relevant concentrations) protective drugs, i.e., Amifostine, WR1065, or Conjugates showed 25-50% enhancements in the CFU formation compared to the Non-treatment control groups. Further improving the concentrations of protective drugs to 4 mM (a non-clinically relevant concentration) did not show CFU enhancements. These results demonstrated that the Conjugate could stimulate the proliferation and differentiation of bone marrow mononuclear cells, which may play a role in myeloprotection.

### Example 20

### In Vitro Proliferation of A549 Cancer Cells Co-Cultured with Chemodrugs and Protective Drugs

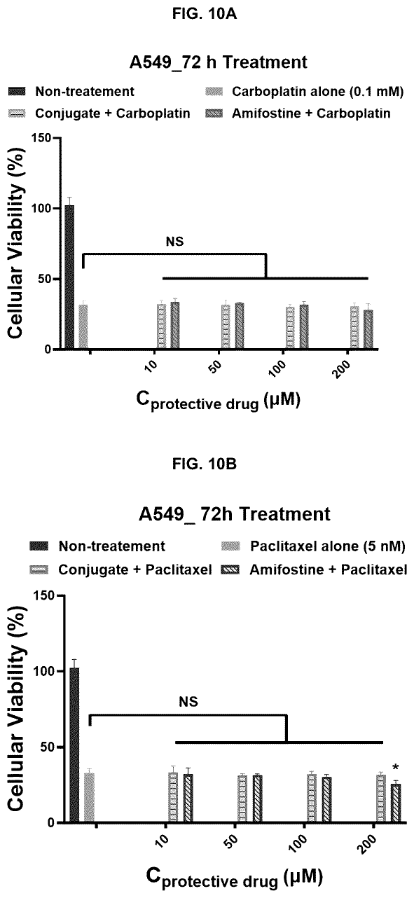

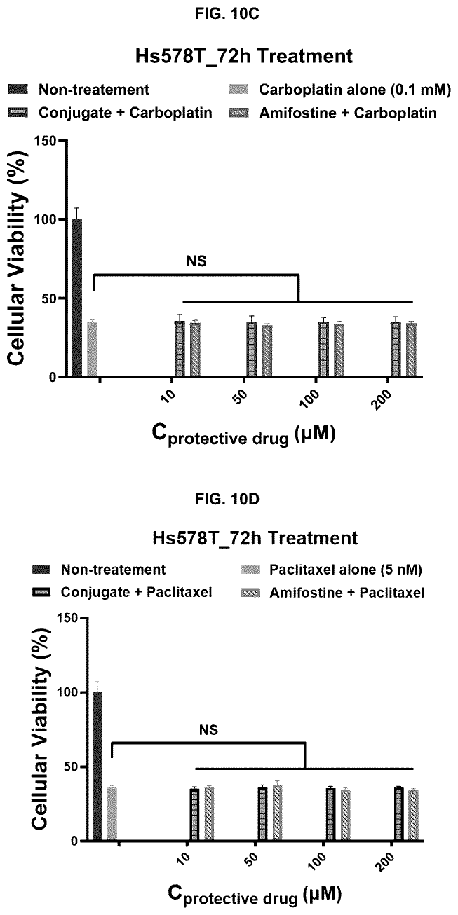

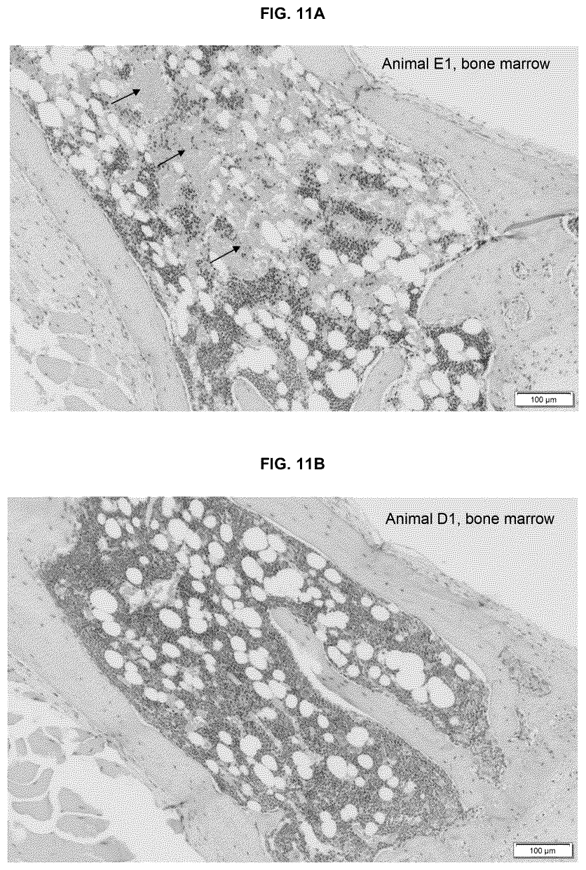

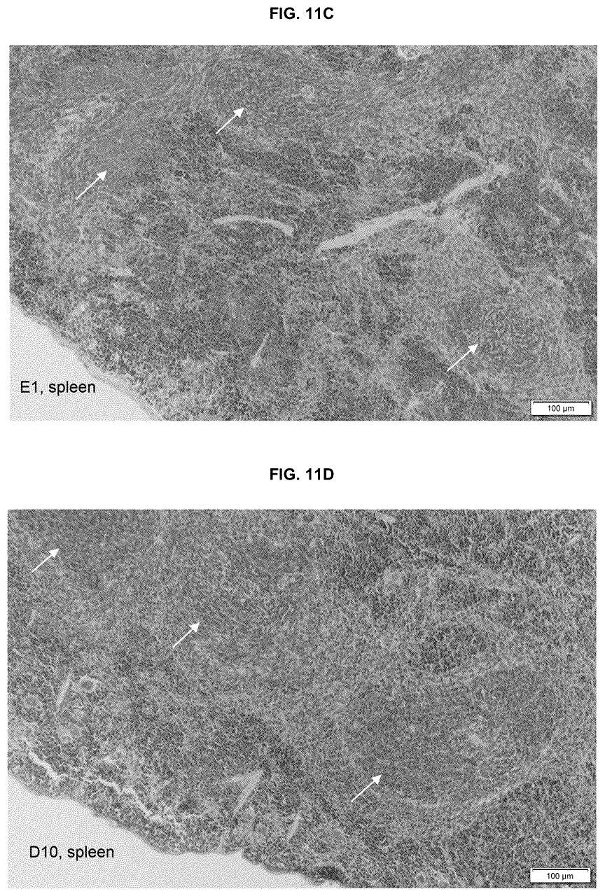

In this exemplary representative experiment, a CCK-8 cellular proliferation assay was conducted for A549 non-small cell lung cancer cells exposed to the Carboplatin with or without protective agents. As shown in FIG. 10A, 72 h of incubation with 0.1 mM Carboplatin significantly reduced cellular proliferation to ˜30% compared to the Non-treatment Control group. For the co-culturing experiments, Conjugate (e.g., the peptide sequence of VYGL (SEQ ID NO: 10)) or Amifostine with concentrations ranging from 10 to 200 μM (non-toxic concentrations to A549 cells) was added into the culture medium 6 h before the addition of Carboplatin. As shown in FIG. 11C, co-culturing cells with Carboplatin and Conjugate or Amifostine resulted in similar proliferation inhibition compared to culturing with Carboplatin alone. This result indicated that Conjugates did not antagonize the anti-tumor effects of Carboplatin for A549 cells.

In another exemplary representative chemoprotection experiment, Paclitaxel was used instead of Carboplatin in the CCK-8 cellular proliferation assay for A549 cells. As shown in FIG. 10B, 72 h of incubation with 5 nM Paclitaxel significantly reduced cellular proliferation to ˜30% compared to the Non-treatment Control group. Comparatively, co-culturing cells with Paclitaxel and Conjugate (e.g., the peptide sequence of VYGL (SEQ ID NO: 10)) or Amifostine led to similar proliferation inhibition compared to culturing with Paclitaxel alone. This result indicated that the Conjugates did not antagonize the anti-tumor effects of Paclitaxel for A549 cells.

### Example 21

### In Vitro Proliferation of Hs578T Cancer Cells Co-cultured with Chemodrugs and Protective Drugs

In this exemplary representative experiment, a CCK-8 cellular proliferation assay was conducted for Hs578T triple-negative breast cancer cells exposed to the Carboplatin with or without protective agents. As shown in FIG. 10C, 72 h of incubation with 0.1 mM Carboplatin significantly reduced cellular proliferation to ˜35% compared to the Non-treatment Control group. For the co-culturing experiments, Conjugate (e.g., the peptide sequence of VYGL (SEQ ID NO: 10)) or Amifostine with concentrations ranging from 10 to 200 μM (non-toxic concentrations to Hs578T cells) was added into the culture medium 6 h before the addition of Carboplatin. As shown in FIG. 11C, co-culturing cells with Carboplatin and Conjugate or Amifostine resulted in similar inhibitory effects compared to culturing with Carboplatin alone. This result indicated that Conjugates did not antagonize the anti-tumor effects of Carboplatin for Hs578T cells.

In another exemplary representative chemoprotection experiment, Paclitaxel was used instead of Carboplatin in the CCK-8 cellular proliferation assay for Hs578T cells. As shown in FIG. 10D, 72 h of incubation with 5 nM Paclitaxel significantly reduced cellular proliferation to ˜35% compared to the Non-treatment Control group. Co-culturing cells with 5 nM Paclitaxel and Conjugate (e.g., the peptide sequence of VYGL (SEQ ID NO: 10)) or Amifostine with concentrations ranging from 10 to 200 μM (non-toxic concentrations) lead to similar proliferation inhibition compared to culturing with Paclitaxel alone. This result indicated that the Conjugates did not antagonize the anti-tumor effects of Paclitaxel for Hs578T cells.

### Example 22

### Pathological Evaluation of Irradiated Mice Injected with a Single Dose of Conjugates

CD2F1 male mice (N=16) were subcutaneously (sc) injected with a single dose of one conjugate drug. For one protective drug conjugate (Conjugate 2), the tested doses were 250, 500, 600, and 700 mg/kg, and the irradiation was conducted at 24 h post-drug administration. The structure of Conjugate 2 included amifostine as the protective drug, alendronate as the bone-targeting ligand, and a protease-cleavable peptide having the sequence of CPLGLYAL (SEQ ID NO: 29) with acetylation at the N-terminus (e.g., AcCPLGLYAL; SEQ ID NO: 76). On day 30, surviving mice were humanely sacrificed and necropsied, eleven tissues of interest (liver, spleen, lung, heart, duodenum, jejunum, ileum, colon, kidney, urinary bladder, and sternum/bone marrow) were collected, preserved in fixative, and processed to glass slides. The completed preparations were stained with hematoxylin and eosin and examined microscopically. Pathological changes in the tissues were graded using a semi-quantitation scale of 0-5, where 0=no change, 1=a minimal change, barely perceptible; 2=a mild change; 3 =a moderate change, affecting up to 50% of the tissue; 4=marked change, affecting up to 90% of the tissue; and 5=severe or maximum change. As shown in Table 1, no pathological change was determined for most tissues examined, while significant changes related to the exposure to 9.2 Gy of TBI were found in the spleen (depletion, white pulp), colon (infiltrate, mixed cell), and sternum bone marrow (atrophy of marrow; and/or hemorrhage in marrow) in the sole surviving animal in the vehicle group. Comparatively, the administration of Conjugate 2 showed significant dose-depending protective effects, especially for bone marrow.

TABLE 1
Pathological Evaluation of tissues for irradiated mice receiving
a single dose of Conjugate 2. Pathological changes were
graded using a semi-quantitation scale of 0-5.

| | | Dose (mg/kg) and # of Surviving Mice on Day 30 | | | | |
|---|---|---|---|---|---|---|
| | | 250 | 500 | 600 | 700 | Vehicle |
| Organ | Pathology | (N = 11) | (N = 12) | (N = 15) | (N = 16) | (N = 1) |
| Liver | — | 0 | 0 | 0 | 0 | 0 |
| Spleen | Depletion, | 2 | 1-2 | 1-2 | 1-2 | 3 |
| | white pulp | | | | | |
| Lung | Infiltrate, | 0 | 0 | 0 | 0 | 0 |
| | mixed cells | | | | | |
| | Fibrosis | 0 | 0 | 0 | 0 | 0 |
| Heart | Infiltrate, | 0 | 0 | 0 | 0 | 0 |
| | mixed cells | | | | | |
| | Mineralization | 0 | 0 | 0 | 0 | 1 |
| Duodenum | — | 0 | 0 | 0 | 0 | 0 |
| Jejunum | — | 0 | 0 | 0 | 0 | 0 |
| Ileum | — | 0 | 0 | 0 | 0 | 0 |
| Colon | Infiltrate, | 1 | 1 | 0-1 | 0-1 | 2 |
| | mixed cells | | | | | |
| Kidney | — | 0 | 0 | 0 | 0 | 0 |
| Urinary | — | 0 | 0 | 0 | 0 | 0 |
| bladder | | | | | | |
| Bone | Atrophy | 2-3 | 2-3 | 2-3 | 2 | 4 |
| Marrow | Hemorrhage | 0-1 | 0-1 | 0-1 | 0 | 3 |

FIG. 11A shows a representative image of bone marrow in the only surviving mouse in the Vehicle Group. The sternal bone marrow was markedly depleted, and the marrow elements (dark-staining cells, not specifically denoted here) are few compared to the bone marrow of mice at this age essentially consisting of a dense, uniform sheet of these dark hematopoietic elements. Moreover, the atrophied marrow (Grade 4) allowed for a collapse of the vascular structure resulting in pools of hemorrhage (black arrows). Comparatively, as shown in FIG. 11B, mice with the administration of 700 mg/kg of Conjugate 2 24 h before the irradiation had a much denser marrow, more dark-staining marrow elements, relatively less marrow fat, and no evidence of vascular collapse, indicating a much better recovery of the bone marrow after the exposure to ionizing radiation. FIG. 11C shows the change of white pulp of the spleen in the surviving mouse in the Vehicle Group, which was much smaller in size and density compared to normal spleen), indicating that a 9.2 Gy TBI exposure resulted in a profound decrease in the rapidly dividing cells of the splenic white pulp germinal centers. Comparatively, the white pulp was essentially normal in the mouse administrated with 700 mg/kg of Conjugate 2 24 h before the irradiation (FIG. 11D), indicating a much better recovery 30 days after the Total Body Irradiation.

TABLE 2
Amino Acid Sequences

| | |
|---|---|
| RR | |
| FR | |
| IPK | |
| KK | |
| RK | |
| LR | |
| VR | |
| AFL | |
| GG | |
| GGG | |
| SSYYSR | SEQ ID NO: 1 |
| SSYYSL | SEQ ID NO: 2 |
| RSSYYSL | SEQ ID NO: 3 |
| PLGL | SEQ ID NO: 4 |
| PIGI | SEQ ID NO: 5 |
| PYSI | SEQ ID NO: 6 |
| PYGI | SEQ ID NO: 7 |
| PYGL | SEQ ID NO: 8 |
| VLSL | SEQ ID NO: 9 |
| VYGL | SEQ ID NO: 10 |
| VLGL | SEQ ID NO: 11 |
| VYSL | SEQ ID NO: 12 |
| PISIY | SEQ ID NO: 13 |
| PSGL | SEQ ID NO: 14 |
| PLGI | SEQ ID NO: 15 |
| PMAL | SEQ ID NO: 16 |
| FPKFFSQ | SEQ ID NO: 17 |
| GFLG | SEQ ID NO: 18 |
| KPIEF | SEQ ID NO: 19 |
| GPLGIAGQ | SEQ ID NO: 20 |
| GPEGIWQ | SEQ ID NO: 21 |
| GGGRR | SEQ ID NO: 22 |
| APIGIAL | SEQ ID NO: 23 |
| APLGLYAL | SEQ ID NO: 24 |
| CKPISIAL | SEQ ID NO: 25 |
| CVLSLYAL | SEQ ID NO: 26 |
| VYGLIAL | SEQ ID NO: 27 |
| GPLSLSAL | SEQ ID NO: 28 |
| CPLGLYAL | SEQ ID NO: 29 |
| CPISIUVI | SEQ ID NO: 30 |
| CQPSGLAL | SEQ ID NO: 31 |
| GPYSIYAL | SEQ ID NO: 32 |
| CKPLGLWAL | SEQ ID NO: 33 |
| CRPMALIIAL | SEQ ID NO: 34 |
| CQPLGALYAL | SEQ ID NO: 35 |
| CKPSGLSLAGI | SEQ ID NO: 36 |
| APSGLYALISL | SEQ ID NO: 37 |
| CQPLGLYAG | SEQ ID NO: 38 |
| CRPLGLWAL | SEQ ID NO: 39 |
| CPISIALGAL | SEQ ID NO: 40 |
| CPYSIYALGL | SEQ ID NO: 41 |
| CKVLGLIAL | SEQ ID NO: 42 |
| CGQVYGLAII | SEQ ID NO: 43 |
| CRVLGLIAL | SEQ ID NO: 44 |
| CFRL | SEQ ID NO: 45 |
| ALRG | SEQ ID NO: 46 |
| CVRGG | SEQ ID NO: 47 |
| CALRGAI | SEQ ID NO: 48 |
| IGFLGIGL | SEQ ID NO: 49 |
| AIGFLGAV | SEQ ID NO: 50 |
| CPGFLGIA | SEQ ID NO: 51 |
| CPFGFLGIAL | SEQ ID NO: 52 |
| CRPGFLGAL | SEQ ID NO: 53 |
| IAGFRGIAK | SEQ ID NO: 54 |
| PGFRKIAG | SEQ ID NO: 55 |
| CPVRIAKG | SEQ ID NO: 56 |
| CQPVRIAL | SEQ ID NO: 57 |
| CQFRVIAL | SEQ ID NO: 58 |
| CKPFRVGAL | SEQ ID NO: 59 |
| CRPAFLGVL | SEQ ID NO: 60 |
| CQPAFLGVL | SEQ ID NO: 61 |
| CIGVRGIAQL | SEQ ID NO: 62 |
| CPIKGAIWL | SEQ ID NO: 63 |
| CGQVYGLAIL | SEQ ID NO: 64 |
| CPLSLYAL | SEQ ID NO: 65 |
| CPLSLYSL | SEQ ID NO: 66 |
| CPLGLAL | SEQ ID NO: 67 |
| CAPIGIAL | SEQ ID NO: 68 |
| CQPLGLWAL | SEQ ID NO: 69 |
| CPISIAL | SEQ ID NO: 70 |
| Ac-CPIKGAIWL* | SEQ ID NO: 71 |
| Ac-CGQVYGLAIL* | SEQ ID NO: 72 |
| Ac-CFRL* | SEQ ID NO: 73 |
| Ac-CKPLGLWAL* | SEQ ID NO: 74 |
| Ac-CQPLGLWAL* | SEQ ID NO: 75 |
| Ac-CPLGLYAL* | SEQ ID NO: 76 |
| Ac-CPFGFLGIAL* | SEQ ID NO: 77 |
| Ac-CKPLGLWA* | SEQ ID NO: 78 |
| Ac-CPISIAL* | SEQ ID NO: 79 |
| Ac-CAPIGIAL* | SEQ ID NO: 80 |

*Ac- indicates an N-terminal acylated peptide.

1. A drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety.

2. The drug conjugate compound of claim 1, wherein at least one of the bone marrow protective drug moieties is a bone marrow protective drug moiety of formula (I):
wherein: 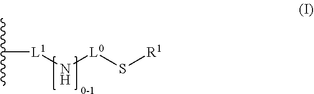
R¹ is hydrogen or —PO₃(R^{1a})₀₋₂, wherein —PO₃(R^{1a})₀₋₂ has a net charge of 0, −1, or −2;
R^{1a}, at each occurrence, is independently hydrogen, Na⁺, K⁺, Ca⁺², Mg⁺², or NH₄⁺;
L⁰ and L¹ are each independently a C₁₋₂₄alkylene wherein optionally 1-8 methylene groups in the C₁₋₂₄alkylene are each independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the C₁₋₂₄alkylene is replaced with —Cy-;
Cy, at each occurrence, is C₃₋₆cycloalkylene, phenylene, a 4- to 6-membered heterocyclylene, or a 4-to 6-membered heteroarylene wherein Cy is optionally substituted with 1-6 substituents independently selected from the group consisting of C₁₋₄alkyl, C₁₋₂fluoroalkyl, and halogen; and
R^{X}, at each occurrence, is independently hydrogen or C₁₋₄alkyl.

3. The drug conjugate compound of claim 2, wherein the bone marrow protective drug moiety of formula (I) is a moiety of formula (I-a):
wherein: 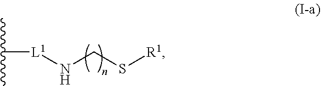
R¹ is hydrogen, —PO₃^{31 2}, —PO₃H⁻, or —PO₃H₂;
L¹ is —C₁₋₄alkylene-N(H)-; and
n is 1-4.

4-7. (canceled)

8. The drug conjugate compound of claim 3, wherein the bone marrow protective drug moiety is

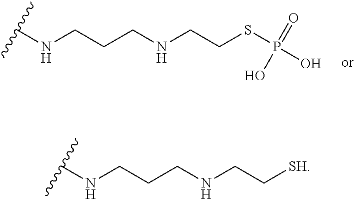 [SMILES: *NCCCNCCSP(=O)(O)O ; "or" ; *NCCCNCCS]

9. The drug conjugate compound of claim 1, wherein the first linker conjugates to the first terminus of the cleavable peptide moiety by a chemical bond selected from the group consisting of an amide bond, an ester bond, a sulfur-carbon single bond, a disulfide bond, a nitrogen-carbon single bond, a nitrogen-carbon double bond, and a nitrogen-nitrogen double bond.

10. The drug conjugate compound of claim 1, wherein the first linker connects to at least one bone marrow protective drug moiety by a linkage selected from the group consisting of

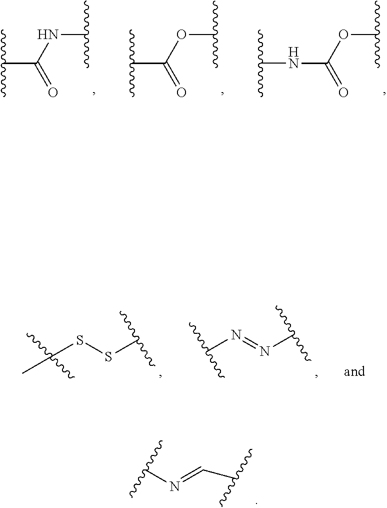

11. The drug conjugate compound of claim 1, wherein the first linker comprises one cleavable chemical bond between one bone marrow protective drug moiety and the first terminus of the cleavable peptide moiety.

12. The drug conjugate compound of claim 11, wherein the cleavable chemical bond is an amide bond between the bone marrow protective drug moiety and the first terminus of the cleavable peptide moiety.

13. The drug conjugate compound of claim 1, wherein the cleavable peptide moiety comprises less than 30 amino acids, and the amino acids are in a linear, cyclic, or branched arrangement.

14. (canceled)

15. The drug conjugate compound of claim 1, wherein the cleavable peptide moiety is a moiety of formula (II-A), (II-B), (II-C) or (II-D): 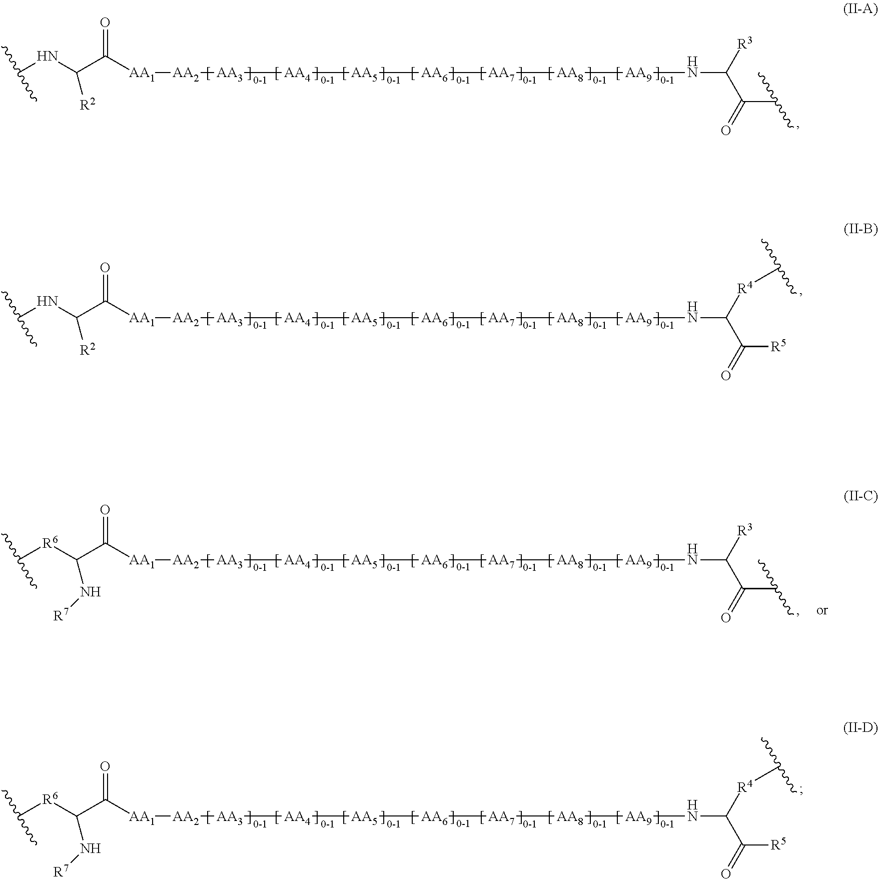
wherein:
AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, Y, P, Q, or V;
AA₈, at each occurrence, is A, I, S, N, V, G, R, M, or Q;
AA₉, at each occurrence, is G, or S, A, L, V, W, Y, N;
R² and R³, at each occurrence, are each independently hydrogen, —CH₃, 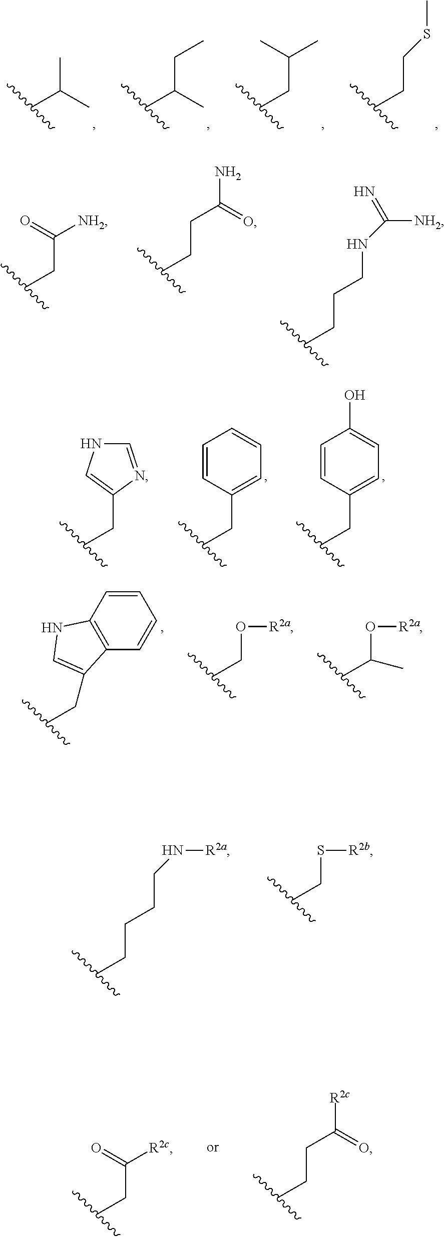
wherein:
R^{2a}, at each occurrence, is hydrogen or 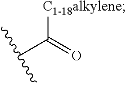
R^{2b}, at each occurrence, is hydrogen, 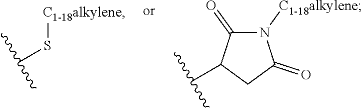
and
R^{2c}, at each occurrence, is hydrogen, 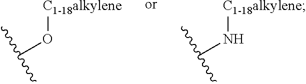
R⁴ and R⁶, at each occurrence, are each independently 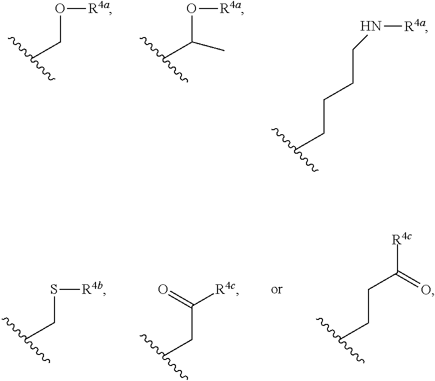
wherein:
R^{4a} is 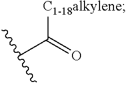
R^{4b} is 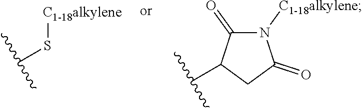
and
R^{4c} is 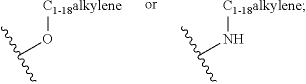
R⁵ is —OH, —O—C₁₋₁₈alkylene, or —NH—C₁₋₁₈alkylene, wherein optionally 1-6 methylene groups in the C₁₋₁₈alkylene are independently replaced with —N(Rx)—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the alkylene of the C₁₋₁₈alkylene is replaced with —Cy-;
R⁷ is hydrogen, or —C(O)—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the C₁₋₁₈alkylene is replaced with —Cy-;
Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and
R^{X}, at each occurrence, is independently hydrogen or C₁₋₄alkyl.

16. The drug conjugate compound of claim 1 wherein the cleavable peptide moiety is a moiety of formula (II-C-A): 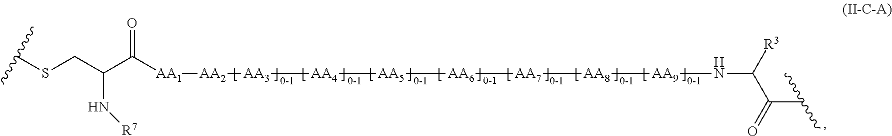
wherein:
AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;
R³ is hydrogen, —CH₃, 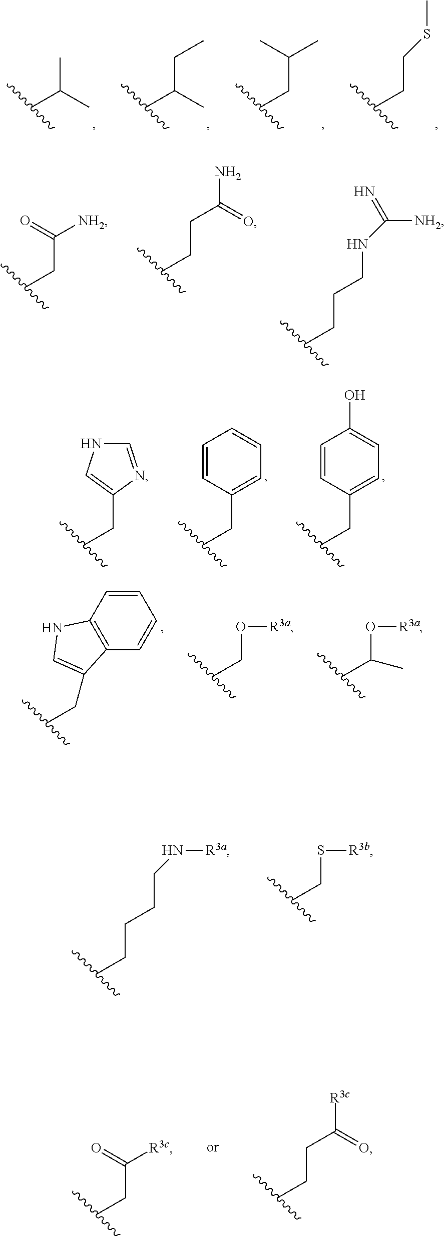
wherein:
R^{3a}, at each occurrence, is hydrogen or 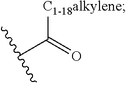
R^{3b}, at each occurrence, is hydrogen, 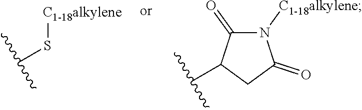
R^{3c}, at each occurrence, is hydrogen, 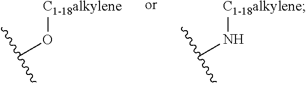
R⁷ is hydrogen, or —C(O)—C₁₋₁₈alkylene, wherein optionally 1-6 methylene groups in the C₁₋₁₈alkylene are independently replaced with —N(R^{X})—, —O—, —S—, —SO—, —SO₂-, or —C(O)-, wherein 2 methylene groups replaced with —N(R^{X})—, —O—, —S—, —SO—, or —SO₂- are separated by two or more carbon atoms in the alkylene and/or optionally 1-4 methylene groups in the C₁₋₁₈alkylene is replaced with —Cy-;
Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and
R^{X}, at each occurrence, is independently hydrogen or C₁₋₄alkyl.

17. The drug conjugate compound of claim 1, wherein the cleavable peptide moiety is a moiety of formula (III): 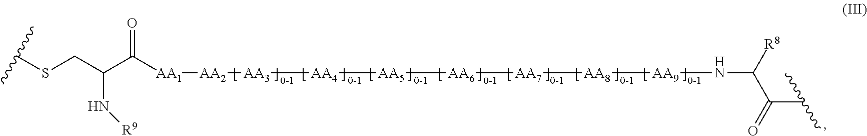
wherein:
AA₁, AA₂, AA₃, AA₄, AA₅, AA₆, AA₇, AA₈, and AA₉ are each an amino acid, wherein:
AA₁ is K, A, V, Y, P, Q, R, G, F, L, W, N, T, S, H, E, or I;
AA₂ is I, L, P, G, Y, S, N, V, G, R, M, A, or Q;
AA₃, at each occurrence, is G, S, L, M, V, F, R, Y, P, Q, or A;
AA₄, at each occurrence, is I, L, S, G, A, Y, F, M, R, or V;
AA₅, at each occurrence, is G, Y, I, U, L, A, S, Y, S, or V;
AA₆, at each occurrence, is S, V, W, I, L, A, Y, M, G, or K;
AA₇, at each occurrence, is V, I, G, L, F, M, A, P, Q, or V;
AA₈, at each occurrence, is A, I, S, N, V, G, M, or Q;
AA₉, at each occurrence, is G, or S, A, L, V, Y, N;
R⁸ is hydrogen, —CH₃, 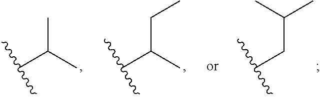
and
R⁹ is hydrogen or —C(O)CH₃.

18. The drug conjugate compound of claim 1, wherein the cleavable peptide moiety is a moiety of formula (III-A) or (III-B):

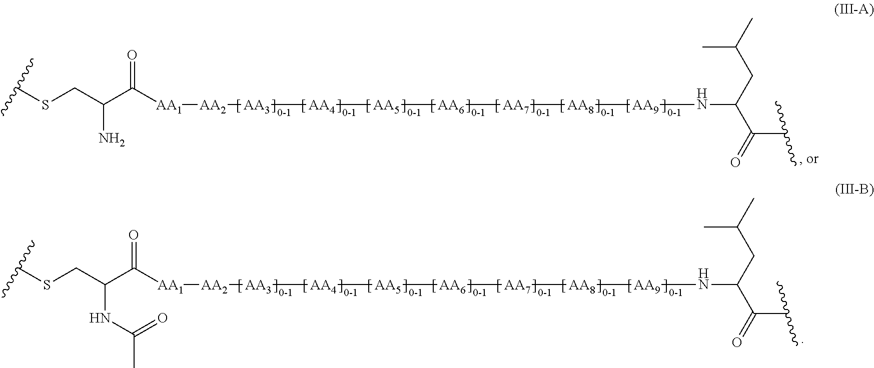

19. The drug conjugate compound of claim 1, wherein the cleavable peptide moiety comprises an amino acid sequence of any one of SEQ ID NO: 1-80.

20. The drug conjugate compound of claim 1, wherein the cleavable peptide moiety comprises at least one sequence of PLGL (SED ID NO: 4), PIGI (SED ID NO: 5), PYSI (SED ID NO: 6), PYGI (SED ID NO: 7), PYGL (SED ID NO: 8), VLSL (SED ID NO: 9), VYGL (SED ID NO: 10), VLGL (SEQ ID NO: 11); VYSL (SED ID NO: 12), PISIY (SED ID NO: 13), PSGL (SED ID NO: 14), PLGI (SED ID NO: 15, or PMAL (SED ID NO: 16).

21. (canceled)

22. The drug conjugate compound of claim 1, wherein the cleavable peptide moiety is cleavable by one or more bone marrow-enriched proteases.

23. The drug conjugate compound of claim 22, wherein at least one bone marrow-enriched protease is a matrix metalloprotease, a disintegrin and metalloproteinase, a cathepsin B protease, a cathepsin K protease, a urokinase plasminogen activator protease, or a tissue-plasminogen activator protease.

24. The drug conjugate compound of claim 1, wherein the bone-targeting ligand moiety is a bisphosphonate moiety of formula (IV): 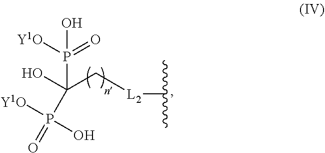
wherein:
Y1, at each occurrence, is independently hydrogen, Na⁺, K⁺, Ca⁺², Mg⁺², or NH₄⁺;
n′ is 1-16;
L² is a C₁₋₂₄alkylene wherein optionally 1-8 methylene groups in the alkylene of L² are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of L² is replaced with —Cy-;
Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and
R^{X}, at each occurrence, is independently hydrogen or C₁₋₄alkyl.

25-26. (canceled)

27. The drug conjugate compound of claim 24, wherein L² comprises —N(H)—, —N(H)—C₁₋₄alkylene-, —N(CH₃)—C₁₋₄alkylene-, phenylene, imidazolylene, or triazolylene.

28. The drug conjugate compound of claim 24, wherein the bone-targeting ligand moiety is

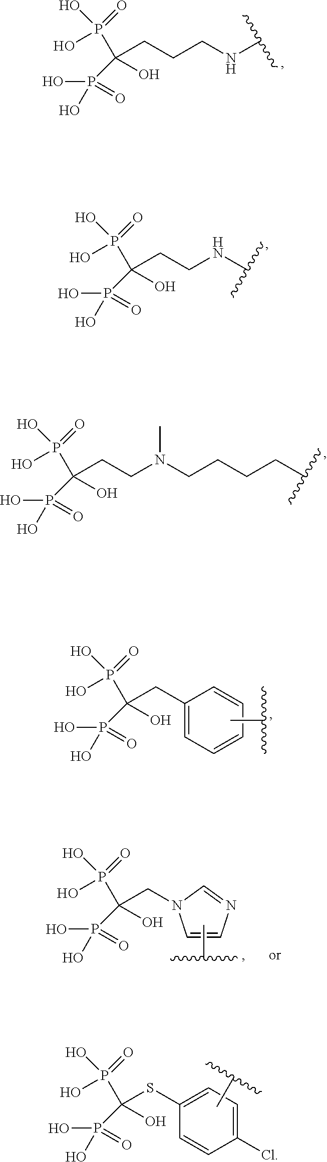

29. The drug conjugate compound of claim 1, wherein the second linker connects to bisphosphonate moiety by a linkage selected from the group consisting of

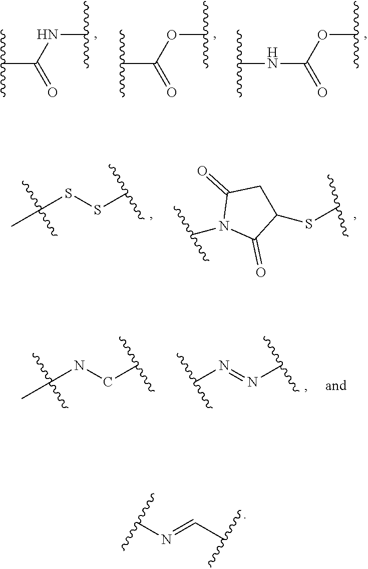

30. The drug conjugate compound of claim 1, wherein the second linker is a —C₁₋₂₄alkylene-wherein optionally 1-8 methylene groups in the second linker are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of the second linker is replaced with —Cy-;
Cy, at each occurrence, is C₃₋₆cycloalkylene, phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and
R^{X}, at each occurrence, is independently hydrogen or C₁₋₄alkyl.

31. The drug conjugate compound of claim 1, wherein the second linker comprises a maleimide moiety, wherein the maleimide moiety is attached to a cysteine moiety that is at least two amino acids away from the first terminus of the cleavable peptide moiety.

32. The drug conjugate compound of claim 1, wherein the second linker is a linker having formula: 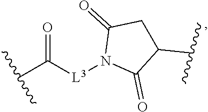
wherein:
L³ is a C₁₋₁₈alkylene wherein optionally 1-6 methylene groups in the alkylene of L³ are independently replaced with —N(R^{X})—, —O—, —S—, —SO—, —SO₂-, or —C(O)-, wherein 2 methylene groups replaced with —N(R^{X})—, —O—, —S—, —SO-, or —SO₂- are separated by two or more carbon atoms in the alkylene and/or optionally 1-4 methylene groups in the alkylene of L³ is replaced with —Cy-;
Cy, at each occurrence, is a C₃₋₆cycloalkylene, a phenylene, a 4- to 6-membered heterocyclylene, or a 4- to 6-membered heteroarylene wherein Cy is optionally substituted with 1-6 substituents independently selected from the group consisting of C₁₋₄alkyl, C₁₋₂fluoroalkyl, and halogen; and
R^{X}, at each occurrence, is independently hydrogen or C₁₋₄alkyl.

33. The drug conjugate compound of claim 33,
wherein L³ comprises repeating units of

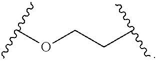

34. The drug conjugate compound of claim 33,
wherein the second linker is:

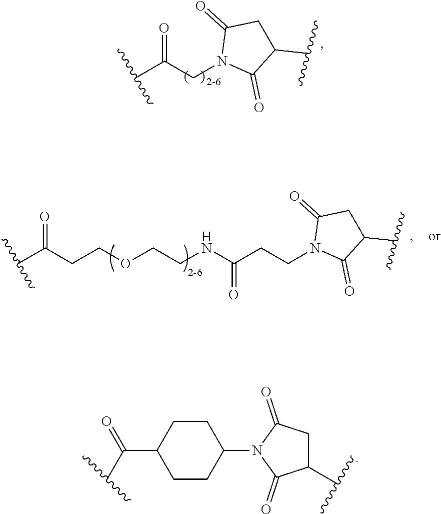

35. A pharmaceutical composition comprising the drug conjugate compound of claim 1 and a pharmaceutically acceptable carrier.

36-37. (canceled)

38. A method of protecting a subject from radiation or chemotherapeutic toxicity, the method comprising:
administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety.

39. A method of treating or preventing a bone marrow disease in a subject, the method comprising:
administering to the subject a pharmaceutical composition comprising a therapeutically effective amount of a drug conjugate compound comprising:
one or more bone marrow protective drug moieties;
a cleavable peptide moiety having a first terminus and a second terminus;
a bone-targeting ligand moiety;
a first linker conjugating the bone marrow protective drug moieties to the first terminus of the cleavable peptide moiety; and
a second linker conjugating the bone-targeting ligand moiety to an amino acid, where the amino acid conjugated to the bone-targeting ligand moiety is at least two amino acids away from the first terminus of the cleavable peptide moiety.

40. The method of claim 39, wherein the bone marrow disease comprises ionizing radiation-induced acute hematopoietic subsyndrome, chemotherapy-caused myelosuppression, radiotherapy-induced myelotoxicity, or combinations thereof.

41-45. (canceled)
